(19) United States
(12) Patent Application Publication
(10) Pub. No.: US 2007/0148136 A1
(43) Pub. Date: Jun. 28, 2007

(54) Methods of using ammonia oxidizing bacteria

(76) Inventor: David R. Whitlock, Watertown, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 10/573,513

(22) PCT Filed: Sep. 27, 2004
(86) PCT No.: PCT/US04/31690

§ 371 Date: Mar. 27, 2006

Related U.S. Application Data
(60) Provisional application No. 60/506,225, filed on Sep. 26, 2003.
(60) Provisional application No. 60/585,652, filed on Jul. 6, 2004.

(51) Int. Cl. A61K 35/74

(52) U.S. Cl. 424/93.4

(57) ABSTRACT

A use of ammonia oxidizing bacteria in the manufacture of a medicament and a method for treating a subject who has developed or is at risk of developing at least one of hypertension, hypertrophic organ degeneration, Raynaud's phenomena, fibrotic organ degeneration, allergies, autoimmune sensitization, end stage renal disease, obesity, diabetes type 1, osteoporosis, impotence, hair loss, cancer, aging, autism, and an autism spectrum symptom comprising positioning ammonia oxidizing bacteria close proximity of a surface of the subject, of nitric oxide and nitric oxide precursors using ammonia oxidizing bacteria.

### FIELD OF INVENTION

The present invention relates to a composition including ammonia oxidizing bacteria to increase production of nitric oxide and nitric oxide precursors on the surface of a subject and methods of using same to slow the progression of aging and treat and prevent hypertension, hypertrophic organ degeneration, Raynaud's phenomena, fibrotic organ degeneration, allergies, autoimmune sensitization, end stage renal disease, obesity, diabetes type 1, impotence, osteoporosis, aging, autism, autism spectrum disorders, hair loss, and cancer with autotrophic ammonia oxidizing bacteria, specifically by administering nitric oxide to a subject.

### BACKGROUND

Living in an industrialized country has many advantages regarding human health. The causes of death in the developed world tend to be the chronic degenerative diseases of aging, heart disease, kidney failure, Alzheimer's, liver failure, and cancer while the major causes of death in the undeveloped world tend to be acute causes such as infection, starvation and war. However, many people living in the undeveloped world have health profiles that seem “better” than their developed world age matched controls. They have a lower body mass index, lower blood pressure, lower incidence of diabetes type 1, less kidney failure, less heart disease, fewer allergies, less autoimmune disease, less Alzheimer's. The difference is equally apparent even within the same country, between urban and rural dwellers, between rich and poor. Many of the differences are especially apparent in those with dark skin. Adult immigrants, born and raised in undeveloped countries, who move to developed countries typically have better health profiles than do their children born and raised in the developed country.

Many of the chronic degenerative diseases of the developed world correlate positively with excess body fat. Obesity worsens the prognosis for virtually every chronic disease. Yet not every obese person gets these diseases, and not everyone with these diseases is obese. Some diseases such as cancer, don't seem to have an “obvious” cause, they seem to strike almost at random. In an earlier age, people would have attributed such diseases to “evil spirits” or “angering the gods.” Now, the “conventional wisdom” is that the “cause” of all of these degenerative diseases is that people do not exercise enough, watch too much TV, eat too many “refined” foods with “too much” fat, sugar, and salt, and are exposed to too many “chemicals”. This is believed to occur in spite of the modern preoccupation with being thin. Changing one's diet by only 100 calories a day will cause one to gain (or lose) about 10 pounds in a year. In the rural undeveloped world, it would seem unlikely that there is virtually no one who has access to an extra 100 calories a day of food. If anything, obesity should be more common in the undeveloped world, because without refrigeration, excess food is best stored by being eaten and stored as fat. Similarly, it is doubtful that every adult who desires to lose weight is so weak-willed that they cannot reduce their intake by 100 calories a day.

The degenerative diseases of the industrialized world which are exacerbated by obesity are leading causes of death. Many of these diseases are characterized by fibrotic organ hypertrophy, including dilative cardiomyopathy, or congestive heart failure, end stage renal disease, systemic sclerosis, and liver cirrhosis. Many billions have been spent trying to prevent and cure these seemingly disparate disorders, yet the numbers of obese individuals whose health is made worse by their obesity is increasing. A method to prevent these degenerative disorders would have major health implications.

Diabetes comprises two disorders, both characterized by elevated blood glucose levels. In diabetes type 1, the pancreatic islets which produce insulin are destroyed, and the body loses the ability to produce insulin. Unless insulin is administered, blood sugar can rise to pathological levels. In diabetes type 2, the body becomes “insulin resistant”, that is, glucose becomes elevated, and increased excretion of insulin by the pancreatic islets does not serve to adequately regulate glucose utilization by the body. Usually, type 2 diabetes precedes type 1, but both can occur simultaneously. In spite of significant morbidity and mortality associated with both types of diabetes, there is no clear understanding of the cause.

Immune system sensitization accompanies many of these same disorders, including primary biliary cirrhosis, diabetes type 1, and systemic sclerosis. Asthma and allergies are common in the developed world and rare in the undeveloped world. The “hygiene hypothesis” suggests that exposure to “dirt”, bacteria or other antigens in early childhood “protects” against immune system deviation in later life. Despite concerted searching, as yet, no such agent has been found.

Autism is a spectrum of sometimes debilitating development disorders. The “cause” remains obscure, but autism often becomes apparent in the first few years of life. It is during this time that the brain is growing rapidly and forming and reforming many new connections. There is some thought that autism occurs when these connections do not form properly. Among 3 to 4 year olds autistic children, B. F. Sparks et al. show that brain volume was 10 to 13% greater than in normal children and in children with development delays that were not autistic. (Sparke et al, Brain structural abnormalities in young children with autism spectrum disorder, Neurology Jul. 23, 2002;59(2):184-92.) Dr. E. H. Aylward, et al. have demonstrated that improper brain growth, and in particular excessive brain volume, has been correlated with autism. (Aylward et al., Effects of age on brain volume and head circumference in autism. Neurology 2002;59:175-183.)

NO is involved in many physiological processes. Because many of the effects of NO are nonlinear and are coupled to many other physiological processes, experimental determination of the effects of NO is not simple, particularly when it is not easy to change basal NO levels. Ragnar Henningsson et al. have indicated that inhibition of NOS with L-NAME can increase NO levels at particular sites. (Henningsson et al., Chronic blockade of NO synthase paradoxically increases islet NO production and modulates islet hormone release, Am J Physiol Endocrinol Metab 279: E95-E107, 2000.)

Thayne L. Sweeten et al. has reported that there is an increased level of NO production in autistic individuals. ( Sweeten et al., High nitric oxide production in autistic disorder: a possible role for interferon-γ, Biological Psychiatry Volume 55, Issue 4, February 2004, Pages 434-437.) Sadik Sogut et al. have also reported higher levels of NO in autistic individuals. (Sogut et al., Changes in nitric oxide levels and antioxidant enzyme activities may have a role in the pathophysiological mechanisms involved in autism, Clinica Chimica Acta 331 (2003) 111-117.) Elevated serum nitrate and nitrite levels are also observed by G. Giovannoni et al. in patients with multiple sclerosis. (Giovannoni et al., Raised serum nitrate and nitrite levels in patients with multiple sclerosis, Journal of the Neurological Sciences 145 (1997) 77-81.)

One researcher, Lennart Gustafsson has suggested that autism might result from low NO due to inadequate levels of nitric oxide synthase. Neural network theory and recent neuroanatomical findings indicate that inadequate nitric oxide synthase will cause autism. (In Pallade V, Howlett R J, Jain L, editors, Lecture notes in artificial intelligence, Volume 2774, part II. New York: Springer-Verlag, P 1109-14.) Gustafsson suggests that the inadequate levels of nitric oxide synthase produces abnormal minicolumn architecture during development, which he suggests might also be produced by low levels of serotonin. (Comment on “disruption in the inhibitory architecture of the cell minicolumns” Implications for autism, Neuroscientist 10 (3): 189-191, Jan. 8, 2004.) He suggests that autism might be treated by increasing the activity of nitric oxide synthase in the brain, but offers no suggestions of how to do so. He notes that a nitric oxide explanation provides a rational for some of the seemingly disparate symptoms observed in autism spectrum disorders including comorbidity with epilepsy, motor impairment, sleep problems, aggression, and reduced nociception.

Osteoporosis is a leading exacerbating factor in fractures in the elderly, The age standardized incidence of low trauma fractures is increasing in elderly populations, with no know explanation. (P. Kannus et. al. Perspective: Why is the age-standardized incidence of low-trauma fractures rising in many elderly populations? Journal of bond and mineral research vol. 17, No. 8, 2002.)

### SUMMARY

One embodiment of the invention is directed to a method of treating a subject who has developed or is at risk of developing at least one of hypertension, hypertrophic organ degeneration, Raynaud's phenomena, fibrotic organ degeneration, allergies, autoimmune sensitization, end stage renal disease, obesity, diabetes type 1, impotence, cancer, osteoporosis, aging, autism, an autism spectrum symptom, and hair loss. The method comprises identifying a subject, and positioning ammonia oxidizing bacteria in close proximity to the subject. In one aspect, the ammonia oxidizing bacteria may be selected from the group consisting of any of Nitrosomonas, Nitrosococcus, Nitrosospira, Nitrosocystis, Nitrosolobus, Nitrosovibrio, and combinations thereof

Another embodiment of the invention is directed to augmenting animal growth comprising removing AAOB from the surface of the animal.

In another embodiment, ammonia oxidizing bacteria is used in the manufacture of a medicament for providing nitric oxide to a subject, wherein said medicament is suitable for being positioned in close proximity to said subject, substantially as described in the specification, wherein the subject has developed or is at risk of developing at least one of: hypertension, hypertrophic organ degeneration, Raynaud's phenomena, fibrotic organ degeneration, allergies, autoimmune sensitization, end stage renal disease, obesity, diabetes type 1, osteoporosis, impotence, hair loss, cancer, autism, an autism spectrum symptom, and reduced health due to aging.

### BRIEF DESCRIPTION OF THE DRAWINGS

FIG. 1 shows a plot of liver enzymes, alanine transaminase levels (SGPT or ALT) for a single individual both before and during application of AAOB to the scalp and body;

FIG. 2 shows the incidence of Alzeheimer's Disease verses minimum temperature during the hottest month for a number of cities;

FIG. 3 shows the number of US patents issued on shampoo verses the year of issue and the number of persons diagnosed with diabetes type 1 verses the year;

FIG. 4 shows NO flux verses NO ppb in sweep gas;

FIG. 5 shows NO in sweep gas verses time;

FIG. 6. shows NO flux verses NO ppb in sweep gas; and

FIG. 7 shows NO from scalp, plethysmograph temperature and volume verses time.

FIG. 8 shows NO from scalp, plethysmograph temperature and volume verses time.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention relates to a composition including ammonia oxidizing bacteria to increase production of nitric oxide and/or nitric oxide precursors in close proximity to a surface of a subject and methods for slowing the progression of aging and treating and preventing hypertension, hypertrophic organ degeneration, Raynaud's phenomena, fibrotic organ degeneration, allergies, autoimmune sensitization, end stage renal disease, obesity, osteoporosis, diabetes type 1, impotence, Autism, Autism spectrum disorders, and cancer with autotrophic ammonia oxidizing bacteria by administering nitric oxide to a subject. “Subject,” as used herein, is defined as a human or vertebrate animal including, but not limited to, a dog, cat, horse, cow, pit, sheep, goat, chicken, primate e.g., monkey, rat, and mouse. The term “treat” is used herein to mean prevent or retard the onset of a disease or disorder as well as to retard or stop the progression of disease or disorder after its onset, or to reduce any symptoms commonly associated with the disorder, even if those symptoms do not reach the threshold for clinical disease.

As used herein, the phrase Autism Spectrum Disorders is defined as is generally recognized, (DSM IV, Diagnostic and statistical manual of mental disorders, 4^{th} ed. Washington, DC: American Psychiatric Association, 1994.) namely Autistic disorder, or Pervasive Development Disorder characterized by severe quantitative deficits in communication, both verbal and non-verbal, social interaction and play, and stereotypical narrow range of interests, Asperger's syndrome, deficient sociability and narrow ranges of interests, and disintegrative disorder, where an otherwise normally developing child severely regresses resulting in severe acquired autism. Examples of Autism Spectrum Disorders include autism, Asperger's syndrome, and Heller's syndrome. Under conventional practice, Autism Spectrum Disorders are limited to fairly severe levels of dysfunction.

Autism is a severe disorder characterized by severe impairment of social interactions. An individual must have multiple and severe deficits to meet the diagnostic criteria for autism. It is to be recognized that many of the attributes of individuals with Autism Spectrum Disorders are observed in other individuals, but to a lesser degree, a degree that does not reach the threshold for clinical Autism or Autism Spectrum Disorders. Symptoms characteristic of Autism Spectrum Disorders that may or may not reach the diagnostic severity in terms of number and/or degree of Autism Spectrum Disorders are defined herein as autism spectrum symptoms. The severity of those autism spectrum symptoms can also be reduced through the method of this invention. A major use of this invention is to reduce the severity of these autistic symptoms, both in individuals with autism and Autism Spectrum Disorders, and in individuals at risk for developing autism or Autism Spectrum Disorders, and in individuals at risk for developing one or more symptoms of Autism Spectrum Disorders.

According to an embodiment of the invention, nitric oxide, a nitric oxide precursor, and/or a nitric oxide releasing compound may be positioned in close proximity to a surface of a subject to slow the progression of aging and treat and prevent hypertension, hypertrophic organ degeneration, Raynaud's phenomena, fibrotic organ degeneration, allergies, autoimmune sensitization, end stage renal disease, obesity, osteoporosis, diabetes type 1, impotence, Autism, Autism Spectrum Disorders, and cancer.

According to one aspect of the invention, it is appreciated that most chronic degenerative diseases of the modern world, as well as obesity and many cancers may be the natural consequence of the body's natural physiological response to modern bathing practices that wash away a substantial amount of previously unknown commensal autotrophic ammonia oxidizing bacteria (AAOB). Accordingly, one aspect of the invention is that these degenerative diseases, Autism, Autism Spectrum Disorders, diabetes type 1, osteoporosis, and obesity may be treated or prevented by applying the AAOB on or in close proximity to a subject. Similarly, another aspect of the invention is that these degenerative diseases may be treated or prevented by not bathing.

More specifically, in one embodiment, applying a composition of an autotrophic ammonia oxidizing bacteria to skin during or after bathing to metabolize urea and other components of perspiration into nitrite and ultimately into Nitric Oxide (NO) results in a natural source of NO. One aspect of the present invention causes topical nitric oxide release at or near the surface of the skin where it can diffuse into the skin and have local as well as systemic effects. This nitric oxide can then participate in the normal metabolic pathways by which nitric oxide is utilized by the body.

Any ammonia oxidizing bacteria may be used in the present invention. In a preferred embodiment, the ammonia oxidizing bacteria may have the following characteristics as are readily known in the art: ability to rapidly metabolize ammonia and urea to nitrite and other NO precursors; non pathogenic; non allergenic; non producer of odoriferous compounds; non producer of malodorous compounds; ability to survive and grow in human sweat; ability to survive and grow under conditions of high salt concentration; and ability to survive and grow under conditions of low water activity. Examples of ammonia oxidizing bacteria include, but are not limited to, Nitroso monas, Nitrosococcus, Nitrosospira, Nitrosocystis, Nitrosolobus, Nitrosovibrio, and combinations thereof, as disclosed in PCT Publication No. WO 03/057380 A2 and PCT Publication No. WO 02/13982 A1, both of which are herein incorporated by reference for all purposes.

Autotrophic ammonia oxidizing bacteria (AAOB) are universally present in all soils and all natural waters, where they perform the first step (oxidation of ammonia to nitrite) in the process of nitrification. NO is a normal minor product of AAOB metabolism when oxidizing ammonia with O₂. Some strains can utilize nitrite or NO₂ as the terminal electron sink, in which cases NO production is increased. AAOB are obligate autotrophs and are unable to grow on media suitable for isolation of pathogens all of which are heterotrophic. AAOB derive all metabolic energy only from the oxidation of ammonia to nitrite with nitric oxide (NO) as an intermediate product in their respiration chain and derive virtually all carbon by fixing carbon dioxide. They are incapable of utilizing carbon sources other than a few simple molecules because they lack the enzyme systems to do so. Autotrophic ammonia oxidizing bacteria (AAOB) are obligate autotrophic bacteria as noted by Alan B. Hooper and A. Krummel at al. (Alan B. Hooper, Biochemical Basis of Obligate Autotrophy in Nitrosomonas europaea, Journal of Bacteriology, February 1969, p. 776-779; Antje Krummel et al., Effect of Organic Matter on Growth and Cell Yield of Ammonia-Oxidizing Bacteria, Arch Microbiol (1982)133: 50-54.) The complete genome of one of them (Nitrosomonas europaea) has been sequenced by Chain et al, and has ˜2460 genes that code for proteins. (Chain et al., Complete Genome Sequence of the Ammonia-Oxidizing Bacterium and Obligate Chemolithoautotroph Nitrosomonas europaea. Journal Of Bacteriology, May 2003, p. 2759-2773.) From an inspection of the genome, it is clear that these bacteria cannot cause disease. There are no genes for toxins or transporters to excrete them or other known virulence factors. They do not possess enzymes to degrade or utilize the complex organic compounds found in animal tissues. They do not grow on any heterotrophic media such as is used for isolating pathogens (all of which are heterotrophic as reported by M Schaechter). (Moselio Schaechter, Gerald Mendoff, David Schlessinger, ed., Mechanisms of Microbial Disease, Williams & Wilkins, Baltimore, Md., USA, 1989.) They are Gram negative bacteria, elicit antibodies, are susceptible to antibiotics, and are killed by ppm levels of linear alkyl benzene sulfonate detergents. They are slow growing with optimum doubling times of 10 hours compared to 20 minutes for heterotrophs.

Natural bacteria can be used as well as bacteria whose characteristics have been altered through genetic engineering techniques. Bacteria culturing techniques can be used to isolate strains with the above characteristics. A mixture of pure strains would avoid the problems associated with simply culturing bacteria from the skin, which includes the potential growth of pathogens and other bacteria having undesirable characteristics. However, culturing bacteria from the skin and growing them on growth media that simulates the composition of human perspiration may also be effective at increasing the nitric oxide production rate. A useful method for culturing and isolating such bacteria is to grow them on media containing urea and ammonia plus mineral salts, but without the organic compounds that heterotrophic bacteria utilize, such as sugars and proteins. When isolating autotrophic ammonia and ammonia oxidizing bacteria, it may also be desirable to attempt growth on a heterotrophic media to verify that the autotrophic strain is not contaminated with heterotrophic bacteria. Nitrobacter are inhibited by elevated pH and by free ammonia. In soil this can lead to the accumulation of nitrite in soil which is quite toxic when compared to nitrate. The skin contains significant xanthine oxidoreductase which reduces nitrite to NO, substantially preventing the accumulation of nitrite. Inhibiting bacteria such as Nitrobacter that reduce the nitrite concentration on the skin is a useful method to further enhance nitric oxide release. Alternatively, Nitrobacter may be included, which will then increase the production of nitrate. Then other bacteria utilizing this nitrate and the other organic compounds on human skin to form nitrite can be used

Bacteria that are useful in this regard are bacteria that metabolize the normal constituents of human perspiration into NO precursors. These include, for example, urea to nitrite, urea to nitrate, nitrate to nitrite, urea to ammonia, nitrite to nitrate, and ammonia to nitrite. In some cases a mixed culture is preferred. The bacteria can conveniently be applied during or after bathing and can be incorporated into various soaps, topical powders, creams, aerosols, gels and salves. One aspect of the invention contemplates application to body parts that perspire the most, such as, for example, hands, feet, genital area, underarm area, neck and scalp. The major difference between these different areas of the skin is the activity of water. The skin of the hands is much drier than that of the feet, normally covered with socks and shoes, due to the increased exposure of the hands to the drying effects of ambient air. It is contemplated that different strains of bacteria may work best on different areas of the body, and a mixed culture of all the types would allow those that grow best to proliferate and acclimate and become the dominant culture present in a specific area. Clothing may also be worn to change the local microclimate to facilitate the growth of the desired bacteria. For example, wearing a hat may simulate dense hair and help to maintain the scalp in a warmer and moister environment.

Because a normal skin environment is relatively dry, bacteria adapted to low water tension environments are advantageous. One example of a moderately halophilic ammonia oxidizing bacteria is Nitrosococcus mobillis described by Hans-Peter Koops, et al. (Arch. Microbiol. 107, 277-282(1976)). This bacteria has a broad range of growth. For example, while the optimum pH for growth is 7.5, at pH 6.5 it still grows at 33% of its maximal rate. Another more halophilic species, Nitrosococcus halophillus described by H. P. Koops, et al. (arch. Micorbiol. (1990) 154:244-248) was isolated from saturated salt solutions in a natural salt lake. Nitrosococcus oceanus (ATCC 1907) is halophilic but has an optimum salt concentration intermediate between the other two. The optimum NaCl concentrations for the three are 200, 700, and 500 mM NaCl respectively. N. oceanus however utilizes urea and tolerates ammonia concentrations as high as 1100 mM as ammonium chloride. While growth at optimum conditions is the fastest, similar results may be achieved by using more bacteria. Thus while the optimum pH for growth of N. mobillis is 7.5, one can achieve the same nitrite production by using 3 times as many bacteria at pH 6.5. Because the quantities of bacteria in the present invention may be large, a number of orders of magnitude larger than that which occurs within 24 hours of bathing, the fact that the pH of the skin is not optimum for these bacteria is not an inhibition to their use. Because N. halophillus was isolated from a saturated salt solution, it should easily survive the relatively moister human skin environment.

Some bacteria produce nitric oxide directly. One example is described in “Production of nitric oxide in Nitrosomonas europaea by reduction of nitrite”, by Armin Remde, et al. (Arch. Microbiol. (1990) 154:187-191). N. europaea as well as Nitrosovibrio were demonstrated to produce nitric oxide directly. Nitrosovibrio is often found growing on rock where the acid generated causes corrosion. It has been suggested by Poth and Focht, “Dinitrogen production from nitrite by a Nitrosomonas isolate.” (Appl Environ Microbiol 52:957-959), that this reduction of nitrite to volatile nitric oxide is used as a method for the organism to eliminate the toxic nitrite from the environment where the organism is growing, such as the surface of a rock.

In order to understand the beneficial aspects of these bacteria, it is helpful to understand angiogenesis. All body cells, except those within a few hundred microns of the external air, receive all metabolic O₂ from the blood supply. The O₂ is absorbed by the blood in the lung, is carried by red blood cells as 0₂ated hemoglobin to the peripheral tissues, where it is exchanged for carbon dioxide, which is carried back and exhaled from the lung. O₂ must diffuse from the erythrocyte, through the plasma, through the endothelium and through the various tissues until it reached the mitochondria in the cell which consumes it. The human body contains about 5 liters of blood, so the volume of the circulatory system is small compared to that of the body. O₂ is not actively transported. It passively diff-uses down a concentration gradient from the air to the erythrocyte, from the erythrocyte to the cell, and from the cell to cytochrome oxidase where it is consumed. The concentration of O₂ at the site of consumption is the lowest in the body, and the O₂ flux is determined by the diffusion resistance and the concentration gradient. Achieving sufficient O₂ supply to all the peripheral tissues requires exquisite control of capillary size and location. If the spacing between capillaries were increased, achieving the same flux of O₂ would require a larger concentration difference and hence a lower O₂ concentration at cytochrome oxidase. With more cells between capillaries, the O₂ demand would be greater. If the spacing between capillaries were decreased, there would be less space available for the cells that perform the metabolic function of the organ.

In one aspect of the invention, it is appreciated that NO from autotrophic ammonia oxidizing bacteria (AAOB) is readily absorbed by the outer skin and converted into S-nitrosothios since the outer skin is free from hemoglobin. M. Stucker et al. have shown that the external skin receives all of its O₂ from the external air in “The cutaneous uptake of atmospheric oxygen contributes significantly to the oxygen supply of human dermis and epidermis. (Journal of Physiology (2002), 538.3, pp. 985-994.) This is readily apparent, because the external skin can be seen to be essentially erythrocyte free. There is circulation of plasma through these layers because they are living and do require the other nutrients in blood, just not the O₂. S-nitrosothiols formed are stable, can diff-use throughout the body, and constitute a volume source of authentic NO and a source of NO to transnitrosate protein thiols.

In another aspect of the invention, it is appreciated that capillary rarefaction may be one of the first indications of insufficient levels of NO. The human body grows from a single cell, and damaged vasculature is efficiently healed in all tissues.

The regulation of angiogenesis and vascular remodeling is the subject of intense research, and a number of factors are well understood.

F. T. Tarek et al. have shown that sparse capillaries, or capillary rarefaction, is commonly seen in people with essential hypertension. (Structural Skin Capillary Rarefaction in Essential Hypertension. Hypertension. 1999;33:998-1001.) Tarek et al. have also shown that capillary rarefaction is seen in people “at risk” for hypertension before they develop it. Rarefaction of Skin Capillaries in Borderline Essential Hypertension Suggests an Early Structural Abnormality. Hypertension. 1999; 34:655-658. There is as yet no good explanation for the cause of capillary rarefaction, but there is both a reduced density of capillaries, and reduced recruitment of capillaries in response to increased local blood demand as noted by E. Serne et al. Impaired Skin Capillary Recruitment in Essential Hypertension Is Caused by Both Functional and Structural Capillary Rarefaction. (Hypertension. 2001;38:238-242.) It is easy to see how capillary rarefaction could lead to hypertension. The metabolic demand of volume of tissue does not go down as the capillary density goes down, so the volumetric blood flow through the sparser network of capillaries must stay the same. With the same volumetric flow but with a reduced cross section available for flow, the pressure drop must increase. It is observed by Greene et al. that microvascular rarefaction does lead to increased pressure drop. (Microvascular rarefaction and tissue vascular resistance in hypertension. Am. J. Physiol. 256 (Heart Circ. Physiol. 25): H126-H131, 1989.) Greene el al. have also shown that with an increased path length for O₂ diffusion from the capillary to the cells farthest from the capillary, the O₂ concentration at those farthest cells must decrease to maintain the same O₂ flux. (Effect of microvascular rarefaction on tissue oxygen delivery in hypertension. Am. J. Physiol. 262 (Heart Circ. Physiol. 31): H1486-H1493, 1992.) In this last reference they show that in addition to greater hypoxia, the heterogeneity of oxic/hypoxic regions is much greater under conditions of capillary rarefaction, and that fluctuation between oxic/hypoxic states increases.

In another aspect of the invention it, is appreciated that it is not merely the concentration of O₂ that affects capillary rarefaction, but also O₂ chemical potential. The O₂ chemical potential is directly proportional to O₂ partial pressure and is proportional to the concentration dissolved in the erythrocyte free plasma and in the extracellular fluid. The chemical potential of O₂ in an erythrocyte is equal to that of the plasma in equilibrium with it. O₂ diffuses from the capillary through the hemoglobin-free tissues to reach the cells that are remote from a capillary.

A number of conditions are associated with the capillary density becoming sparser. Hypertension has been mentioned earlier, and researchers reported that sparse capillaries are also seen in the children of people with essential hypertension, and also in people with diabetes. Significant complications of diabetes are hypertension, diabetic nephropathy, diabetic retinopathy, and diabetic neuropathy. R, Candido et al. have found that the last two conditions are characterized by a reduction in blood flow to the affected areas prior to observed symptoms. (Haemodynamics in microvascular complications in type 1 diabetes. Diabetes Metab Res Rev 2002; 18: 286-304.) Reduced capillary density is associated with obesity, and simple weight loss increases capillary density as shown by A Philip et al. in “Effect of Weight Loss on Muscle Fiber Type, fiber Size, capilarity, and Succinate Dehydrogenase Activity in Humans. The Journal of Clinical Endocrinology & Metabolism Vol. 84, No. 11 4185-4190, 1999.

Researchers have shown that in primary Raynaud's phenomena (PRP), the nailfold capillaries are sparser (slightly) than in normal controls, and more abundant than in patients that have progressed to systemic sclerosis (SSc). M. Bukhari, Increased Nailfold Capillary Dimensions In Primary Raynaud|S Phenomenon And Systemic Sclerosis. British Journal Of Rheumatology Vol 24 No 35: 1127-1131, 1996. They found that the capillary density decreased from 35 loops/mm2 (normal controls) to 33 (PRP), to 17 (SSc). The average distance between capillary limbs was 18μ, 18μ, and 30μ for controls, PRP and SSc.

In another aspect of the invention, it is appreciated that the mechanism that the body normally uses to sense “hypoxia” may affect the body's system that regulates capillary density. According to this aspect of the invention, a significant component of “hypoxia” is sensed, not by a decrease in O₂ levels, but rather by an increase in NO levels. Lowering of basal NO levels interferes with this “hypoxia” sensing, and so affects many bodily functions regulated through “hypoxia.” For Example, anemia is commonly defined as “not enough hemoglobin,” and one consequence of not enough hemoglobin is “hypoxia”, which is defined as “not enough O2.” According to one aspect of the invention, these common definitions do not account for the nitric oxide mediated aspects of both conditions.

At rest, acute isovolemic anemia is well tolerated. A 2/3 reduction in hematocrit has minimal effect on venous return PvO₂, indicating no reduction in either O₂ tension or delivery throughout the entire body. (Weiskopf et al., Human cardiovascular and metabolic response to acute, severe isovolemic anemia, JAMA 1998, vol 279, No.3, 217-221.) At 50% reduction (from 140 to 70 g Hb/L), the average PvO₂ (over 32 subjects) declined from about 77% to about 74% (of saturation). The reduction in O2 capacity of the blood is compensated for by vasodilatation and tachycardia with the heart rate increasing from 63 to 85 bpm. That the compensation is effective is readily apparent, however, the mechanism is not. A typical explanation is that “hypoxia” sensors detected “hypoxia” and compensated with vasodilatation and tachycardia. However, there was no “hypoxia” to detect. There was a slight decrease in blood lactate (a marker for anaerobic respiration) from 0.77 to 0.62 mM/L indicating less anaerobic respiration and less “hypoxia.” The 3% reduction in venous return PvO₂ is the same level of “hypoxia” one would get by ascending 300 meters in altitude (which from personal experience does not produce tachycardia). With the O₂ concentration in the venous return staying the same, and the O₂ consumption staying the same, there is no place in the body where there is a reduction in O₂ concentration. Compensation during isovolemic anemia may not occur because of O₂ sensing.

“Hypoxia” from other causes does not have the same effect on cardiac output. Murray et al. have shown that when a portion of a dog's normal erythrocytes are replaced with erythrocytes that are fully oxidized to metHb, “hypoxic” compensation is minimal. (Circulatory effects of blood viscosity: comparison of methemoglobinemia and anemia, Journal Of Applied Physiology Vol.25, No. 5, 594-599 November 1968.) While maintaining the same hematocrit Hct (43%) and substituting (0, 26, 47%) fully metHb erythrocytes, the cardiac output (CO) declined (178, 171, 156 mL/m/kg) while the arterial PaO₂ (93, 87, 84 mmHg) and PvO₂ (55, 46, 38) also declined. In contrast, when acute isovolemic anemia (Hct 40, 30, 22) was induced using plasma, compensation was much better, CO (155, 177, 187), PaO₂ (87, 88, 91), and PvO₂ (51, 47, 42). When anemia was induced using dextran solution (Hct 41, 25, 15) cardiac output (143, 195, 243), PaO₂ (89, 92, 93), PvO₂ (56, 56, 51) compensation was better still.

As part of their experiments with the metHb tests, a final dilution was done with dextran to lower the Hct to 26% while still maintaining 47% methb. Compensation was much improved with CO (263 mL/m/kg), PaO2 (86 mmHg), and PvO₂ (41 mmHg) all were increased, despite lower Hct, greater O₂, and less “hypoxia.” The compensatory mechanisms to deal with this “hypoxia” may not be due to reduced O₂ levels because the O₂ levels were not reduced, in fact, the O₂ levels were increased.

Deem et al, have reported that pulmonary gas exchange efficiency improves during isovolemic anemia, and exhaled NO increases as Hct decreases (in rabbits). (Mechanisms of improvement in pulmonary gas exchange during isovolemic hemodilution. J. Appl. Physiol. 83: 240-246, 1997.)

As Hct was decreased by dilution with hydroxyethyl starch (30, 23, 17, 11%), cardiac output rose (0.52, 0.60, 0.70, 0.76 L/min), and exhaled NO levels rose (30, 34, 38, 43 nL/min).

Calbet et al. have shown that maximum O₂ consumption (VO2max) is reduced at high altitude, and this reduced VO₂max is not restored by acclimatization. (Why is VO₂ max after altitude acclimatization still reduced despite normalization of arterial O₂ content?, Am J Physiol Regul Integr Comp Physiol 284: R304.R316, 2003.) Koskolou et al. have shown that VO₂max is decreased when hematocrit is decreased in spite of no difference in PaO2 or PvO₂. (Cardiovascular responses to dynamic exercise with acute anemia in humans. Am. J. Physiol. 273 (Heart Circ. Physiol. 42): H1787-H1793, 1997.)

In this last reference, Koskolou et al.'s data clearly show a 17% reduction in maximum work, with Hb change (154.4 to 123.3 g/L) a PaO2 change ( 19.2 to 115.1 mmHg) and a PvO₂ change (23.6 to 23.0 mmHg). Koskolou et al. do not have an explanation for the inability of the trained muscle to “extract” the O₂ which is being delivered by the blood, or the inability of the heart to deliver more blood despite reserve cardiac capacity. This behavior may be explained by the interaction of NO with heme proteins and the competitive inhibition of cytochrome oxidase by NO causing reduced VO2max.

Horses when treated with the NOS inhibitor L-NAME showed an accelerated increase in VO₂ and a lower “O₂ debt”, but also a slightly lower VCO₂max as reported by Casey et al. in “Effect of L-NAME on oxygen uptake kinetics during heavy-intensity exercise in the horse.” (J Appl Physiol 91: 891-896, 2001.) The accelerated VO₂ was attributed to reduced NO inhibition of mitochondrial respiration, and the slightly reduced VCO₂max (62.5, 61.0 L/min) to the reduced cardiac output (which was reduced 12% due to vasoconstriction) observed in the L-NAME group. The increased VO₂max observed with increases in Hct is as in “blood doping” is likely due to decreased NO as well. These examples are all consistent with NO inhibition of mitochondrial respiration and that inhibition being modulated by changes in hematocrit.

Hb is well known to remove NO from solution with kinetics that are first order in both Hb and NO. At steady state, the NO production rate will be constant, and the production rate equals the destruction rate (no accumulation). A sudden drop in hematocrit by 50% will result in an increase in NO concentration because the production rate would continue to equal the destruction rate and as the destruction rate is first order in both NO and Hb it is their product that remains constant. The reaction between NO and Hb is so fast, that the new NO concentration will be reached virtually as soon as the blood and the diluent mix and pass by a vessel wall.

Thus the vasodilatation that is observed in acute isovolemic anemia may be due to the increased NO concentration at the vessel wall. NO mediates dilatation of vessels in response to shear stress and other factors. No change in levels of NO metabolites would be observed, because the production rate of NO is unchanged and continues to equal the destruction rate. The observation of no “hypoxic” compensation with methb substitution can be understood because methb binds NO just as Hb does, so there is no NO concentration increase with metHb substitution as there is with Hb withdrawal.

Many details of NO chemistry while well known are not universally well appreciated. The ligands O₂, CO, H₂S and HCN, along with NO, all bind to heme and may at times be significant in human physiology. The activity of all proteins containing heme (and there are many) will therefore be affected by the concentrations of all of these species. Sometimes, one or several can be ignored, but the circumstances under which a potential activating species can be ignored must be well considered because the binding constants for NO, CO, H₂S, and HCN are many orders of magnitude greater than that of the most abundant ligand, O₂. The various heme containing proteins don't “sense” any of these ligands independently; they only “sense” relative concentrations of all the ligands.

The behavior of NO and NOS enzymes in the body are complex. The gene for one isoform NNOS is, “the most structurally diverse human gene described to date in terms of promoter usage”. (Y. Wang et al., RNA diversity has profound effects on the translation of neuronal nitric oxide synthase. PNAS Oct. 12, 1999 vol. 96 no. 21 12150-12155.) NO is difficult to measure, is active at very low levels, is labile, reactive, and diffuses rapidly, so concentrations change rapidly in time and space. It is active at many diverse sites where it serves diverse signaling and regulatory functions through multiple mechanisms. It is responsible for regulation of vascular tone through cGMP mediated relaxation of smooth muscle. It is responsible for regulation of O₂ consumption by cytochrome oxidase by competitively inhibiting O₂ binding. It is responsible for inhibition of proteases, including caspases, by S-nitrosylation of cysteine residues and induces expression of matrix metalloproteinases. NO is a major component of the immune reaction, and is produced in large quantities by iNOS in response to infection. It should also be recognized that the length scale over which NO gradients are important extends to individual cells. It should also be recognized that not all “NO effects” are mediated through “free No”. S-nitrosothiols can transnitrosate protein thiol groups without free NO ever being present. The state of the art in NO measurement does not allow measurement on the time, distance and concentration scales that are known to be important. With this level of complexity and experimental difficulty, it is not surprising that the details of how NO interacts with hemoglobin (which is perhaps the best understood human protein) are not agreed upon by those most knowledgeable in the field.

It is known that Nitric oxide plays a role in many metabolic pathways. It has been suggested that a basal level of NO exerts a tonal inhibitory response, and that reduction of this basal level leads to a dis-inhibition of those pathways. Zanzinger et al. have reported that NO has been shown to inhibit basal sympathetic tone and attenuate excitatory reflexes. (Inhibition of basal and reflex-mediated sympathetic activity in the RVLM by nitric oxide. Am. J. Physiol. 268 (Regulatory Integrative Comp. Physiol. 37): R958-R962, 1995.)

One function of NO is to regulate O₂ consumption by cytochrome oxidase by binding to cytochrome oxidase and competitively inhibiting the binding of O₂. Inhibition of O₂ consumption is advantageous because the concentration of O₂ at each mitochondria in every cell cannot be well controlled. As O₂ is consumed, the O₂ level drops, more NO binds, and the inhibition increases, slowing the consumption of the remaining O₂. Without this inhibition, the mitochondria closest to the O₂ source would consume more, and those far away would get little or no O₂. For some tissues, such as heart muscle, the O₂ consumption can change by a factor of more than 10 between basal and peak metabolic activity. To achieve this O₂ flux, the gradient must increase because the capillary spacing does not change with O₂ consumption (although there is some increased recruitment of capillaries which were otherwise empty). Decreasing NO concentrations increase the rate of O₂ consumption by mitochondria by removing the inhibition that NO produces.

The inhibition of cytochrome oxidase by NO may depend on the relative concentrations of both NO and O₂. Thus the reduction of VO₂max during hypobaric hypoxia may be due to less O₂ relative to the same NO while the reduction of VO₂max during isovolemic anemia may be due to increased NO relative to the same O₂. The increase in exhaled NO during isovolemic anemia is due to less trapping and destruction in the lung of NO produced in nasal passages. The reduced O₂ delivery to muscle during isovolemic anemia is due to greater NO levels. With greater NO concentration, the operating point of the mitochondria is shifted to a higher O₂ concentration. The concentration of O₂ at the mitochondria is actually increased during isovolemic anemia due to greater inhibition by NO. With higher concentration at the O₂ sink, the concentration gradient is less and so the O₂ flux is less. The reduction in blood lactate during isovolemic anemia demonstrates that the mitochondria may actually be less hypoxic, so anaerobic glycolysis is less. The adverse consequence of decreased NO levels leading to increased anaerobic glycolysis will be discussed later.

Reductions in VO₂max can be observed in hypobaric hypoxia and isovolemic anemia, and VO₂max increases are observed with L-NAME inhibition. This demonstrates that the NO concentration at the mitochondria is coupled to the hemoglobin concentration in the blood by destruction of NO by hemoglobin and to NO production by NOS.

NO binds to the heme of many proteins. Because most of the body's iron is in hemoglobin, the concentration of heme in the blood is much higher than in any other tissue, so the binding of NO by heme will be most rapid there and the blood is considered to be the major sink of NO. A major source of NO is the endothelium where eNOS is constitutively expressed. With the source of NO and the sink of NO so close together, the NO concentration at regions remote from the source and sink will be sensitively dependant on the details of the source-sink interactions. There are other sources of NO as well. Stamler et al. have reported that blood and plasma contains a number of S-nitrosothiols of which the major one is S—NO-albumin. (Nitric oxide circulates in mammalian plasma primarily as an S-nitroso adduct of serum albumin. Proc. Natl. Acad. Sci. USA vol. 89, 764-7677, 1992.)

NO can be cleaved from S-nitrosothiols with light, and by various enzymes including xanthine oxidase, copper ions and copper containing enzymes including Cu,Zn SOD. Many of the metabolic functions of NO do not require liberation of free NO. When a cysteine in the active region of a protein is S-nitrosylated, the activity of the protein is affected. Transfer of NO from one S-nitrosothiol to another is termed transnitrosation, and is catalyzed by a number of enzymes including protein disulfide isomerase. Many of the metabolic effects of NO are known to be mediated through S-nitrosothiols, for example S-nitrosothiols mediate the ventilatory response to hypoxia.

In the example of a 50% reduction in hematocrit, the NO concentration at the capillary wall will increase to match the prior destruction rate, and may double. NO will also passively diffuse throughout the body, and with the major sink being the hemoglobin in the blood, the concentrations elsewhere will increase too. It should be noted, that with the sink being the hemoglobin, the minimum NO concentration occurs at the site of consumption, the hemoglobin in the blood. Thus there will naturally be a gradient of NO concentration that is the reverse of the O₂ gradient, provided there is a source of NO in the peripheral tissues. Although NOS is expressed in many tissues, such a source has not been reported (probably largely due to the experimental difficulty of measuring NO gradients between capillaries).

In one aspect of the invention, it is appreciated that one component of this volume source of NO is low molecular weight S-nitrosothiols produced in the erythrocyte free skin from NO produced on the external skin by autotrophic ammonia oxidizing bacteria. These low molecular weight S-nitrosothiols are stable for long periods, and can diffuse and circulate freely in the plasma. Various enzymes can cleave the NO from various S-nitrosothiols liberating NO at the enzyme site. It is the loss of this volume source of NO from AAOB on the skin that leads to disruptions in normal physiology. The advantage to the body of using S-nitrosothiols to generate NO far from a capillary is that O₂ is not required for NO production from S-nitrosothiols. Production of NO from nitric oxide synthase (NOS) does require O₂. With a sufficient background of S-nitrosothiols, NO can be generated even in anoxic regions. Free NO is not needed either since NO only exerts effects when attached to another molecule, such as the thiol of a cysteine residue or the iron in a heme, so the effects of NO can be mediated by transnitrosation reactions even in the absence of free NO provided that S-nitrosothiols and transnitrosation enzymes are present.

In another embodiment of the invention, it is appreciated that in the absence of overt anoxia, elevated NO may be a more effective “hypoxia” signal to regulate hematocrit and other “hypoxia” mediated factors, than depressed O₂. Since the “normal” hematocrit set point is determined in the absence of overt hypoxia, the “normal” Hct setpoint may be determined by NO and not O₂ levels, or more precisely, by the ratio of NO to O₂ (NO/O₂). The “hypoxia” signal need not be linear with NO/O₂, but the “hypoxia” signal may increase with increased NO and may increase with decreased O₂. Each may have an effect on the “hypoxia” signal, but not necessarily an equal effect.

Similarly, the vascular remodeling that normally occurs continuously and in the absence of overt anoxia must also be regulated through a “hypoxia” signal that also occurs continuously and in the absence of overt anoxia. When blood flow to a capillary bed is reduced, O₂ delivery to portions of the tissue served by that bed is reduced. This results in the heterogeneous appearance of hypoxia, with the cells farthest (in the sense of O₂ diffusion resistance) from the capillaries experiencing hypoxia first. This has been observed in vitro, where perfused rat hearts were infused with a Pd porphine which has its fluorescence quenched by O2, and the fluorescence of the Pd porphine and the fluorescence of NADH (a measure of mitochondria deoxygenation) were observed by Ince et al. during normoxic and hypoxic perfusion. (Heterogeneity of the hypoxic state in rat heart is determined at capillary level. Am. J. Physiol. 264 (Heart Circ. Physiol. 33): H294-H301, 1993.) During the transition from anoxic to normoxic conditions, the regions that had less O₂ matched those that had greater NADH, and the length scale of the heterogeneity of those regions matched that of the capillaries. The literature demonstrates that “hypoxia” is a local effect, it is heterogeneous at the capillary level, that heterogeneity is due to capillary spacing, and that “hypoxia” due to stopped flow has the same heterogeneity as “hypoxia” due to anoxic fluid at high flow. The greatest heterogeneity was observed during recovery from anoxia. It should also be noted that in the absence of sufficient NO, the activity of cytochrome oxidase for O₂ is greater, that is the activity at a given O₂ concentration is greater. Thus cells in close proximity to capillaries will consume more O₂ leaving even less for cells far from a capillary. Insufficient NO will exacerbate the degree of heterogeneity of hypoxia, and will therefore increase the number of transitions between hypoxic and oxic conditions. The production of superoxide is greatest during reoxygenation following hypoxia. The mitochondria respiration chain becomes fully reduced, and O₂ captures the electron before it can be shuttled to cytochrome oxidase. With a reduced NO level, the operating point of the mitochondria is shifted to a lower O₂ concentration. This means that there is less “capacitance” due to O₂ stored in the tissues. More superoxide gets produced, and because superoxide destroys NO with diffusion limited kinetics, more superoxide means even less NO. This destruction of NO by superoxide caused by local hypoxia may exacerbate conditions of insufficient perfusion.

The O₂ partial pressure of the blood is normally quite constant and very well regulated. In order to regulate the spacing of capillaries, the body must measure the diffusion resistance of O₂ to that site and generate capillaries where the O₂ diffusion resistance is too high, and ablate capillaries where the resistance is too low. The O2 demand of tissues fluctuates with their metabolic activity, and the “normal” capillary spacing must be sufficient for “normal” metabolic demand (plus some reserve). The simplest way that O₂ diffusion resistance can be determined and hence regulated is to decrease supply at constant demand. The alternative, increasing demand at constant supply, would require a method to dissipate the metabolic heat that would be liberated, which is not observed. Since the demand must exceed the supply, a “hypoxic” state must be induced, at which time normal functionality must be compromised (otherwise it wouldn't be hypoxia). Decreasing the O₂ concentration or flow rate of blood, while maintaining basal metabolic load, would induce a state of hypoxia and so allow cells to determine the diffusion resistance of O₂. Since metabolic functionality is necessarily compromised, a preferred time to do this would be when metabolic demand is at a minimum, when the organism is not moving or needing to evade predators, such as during sleep. Inducing hypoxia at the lowest metabolic rate also results in the longest time constant, which minimizes the chance of overshoot and hypoxic damage.

Erythropoiesis is mediated in part through erythropoietin (EPO), which is produced primarily by the kidney in response to “hypoxic” stimuli, including hypobaric hypoxia, isovolemic anemia, cobalt chloride, and deferroxamine. Many of the effects of “hypoxia” are mediated through hypoxia-inducible factor (HIF-1α) which activates transcription of dozens of genes including the EPO gene. Complex behavior of HIF-1α in response to NO exposure has been demonstrated by Britta et al, by using authentic NO, NO donors and also transfected cells expressing INOS as NO sources. (Accumulation of HIF-1α under the influence of nitric oxide, Blood 2001; 97: 1009-1015.)

Sandau et al. found that lower NO levels induced a more rapid response and produced more HIF-1α than did higher levels. The only NO donor tested which did not induce HIF-1α was sodium nitroprusside which also releases cyanide. They also determined that the induction of HIF-1α was not mediated through cGMP. Kimura et al, have shown that Angiogenesis is mediated in part through VEGF, which is induced by HIF-1α which is induced by NO. (Hypoxia response element of the human vascular endothelial growth factor gene mediates transcriptional regulation by nitric oxide: control of hypoxia-inducible factor-1 activity by nitric oxide, Blood, 2000; 95: 189-197.) Transcription of enzymes necessary for glycolytic production of ATP occurs in response to HIF-1α. Insufficient NO will then lead to insufficient levels of glycolytic enzymes as well.

Frank et al. have shown that the angiogenesis that accompanies normal wound healing is produced in part by elevated VEGF which is induced by increased nitric oxide. (Nitric oxide triggers enhanced induction of vascular endothelial growth factor expression in cultured keratinocytes (HaCaT) and during cutaneous wound repair, FASEB J. 13, 2002-2014 (1999).)

Thus, when hypoxia is not accompanied by sufficient NO, a lower level of O₂ for a longer period of time is required to elicit induction of HIF-1α and VEGF. It should be remembered that with low NO levels, mitochondrial consumption of O₂ is faster, so the O₂ level will drop faster and farther and for a longer period of time than with high NO.

According to another embodiment of the invention, it is appreciated that accelerated turnover of organ cells by hypoxia induced by capillary rarefaction may be a factor in the accelerated aging that is observed in the chronic degenerative diseases. The body controls spacing between capillaries so as to match the local O₂ demand with the local blood supply. To do this, it induces a state of “hypoxia” and, through HIF-1α and VEGF, initiates angiogenesis where needed. To ensure that the capillaries are not too close, there may also be a signal indicating an absence of nearby “hypoxia” which may lead to capillary ablation, through endothelial cell apoptosis. This ablation may be mediated through the absence of VEGF (or other endothelial cell survival factors) diffusing from “hypoxic” cells nearby. Lang et al. have reported that VEGF deprivation does induce apoptosis in endothelial cells. (VEGF deprivation-induced apoptosis is a component of programmed capillary regression, Development 126, 1407-1415 (1999).) Insufficient VEGF, due to low basal NO, from cells that have insufficient O₂ but which don't have the NO/O₂ ratio to initiate HIF-1α prevents new capillaries from being formed and ablates already formed nearby capillaries by depriving them of VEGF. Thus low basal NO may induce a state of chronic insufficient O₂ in that population of cells farthest from the capillaries, and may increase the average spacing between capillaries. The number of cells that may be affected at any one time is small, and may occur in isolated regions with lengths scales less than the capillary spacing. Moreover, cells may be affected only one at a time. Such an isolated hypoxic cell would be difficult to detect. When such a cell dies through apoptosis or necrosis, the resulting inflammation would also be difficult to detect. Over time, affected cells would die and be cleared, the geometry of the capillary structure would collapse, new cells would move into the hypoxic zone, more capillaries would ablate, and over many years, many of the cells of an organ could be affected. If surviving cells divide to replace the ones that die, the cycle of cell death and cell replacement could occur many times, and over many years the number of so affected cells could exceed the total in the organ, perhaps even by many fold. With each cell division, the telomeres in the cell become shorter, and when the telomeres become too short, the cell can no longer divide.

According to an embodiment of the invention, it is appreciated that capillary rarefaction can then be seen as the consequence of too little NO at cells remote from a capillary. Without enough NO, the cells may not produce the signal to initiate angiogenesis. In spite of chronic low O₂, without enough NO there is no “hypoxic” signal to initiate angiogenesis. However, cells require O₂ for oxidative phosphorylation to supply the ATP and other species needed to perform the various metabolic functions. With inadequate O₂, cell function will be degraded. It should be noted that in the absence of sufficient NO, the O₂ gradient (dO₂/dx) is steeper due to the lack of inhibition of cytochrome oxidase at low O₂. Thus cells that are beyond the NO/O₂ threshold for inducing angiogenesis may experience greater hypoxia induced dysfunction. Some cells can generate ATP through anaerobic glycolysis. However, anaerobic glycolysis consumes 19 times more glucose than does aerobic glycolysis per unit of ATP generated. If even a few cells are producing ATP through anaerobic glycolysis, the local glucose concentration may become depleted. The effect of this localized depletion in glucose levels due to hypoxia will be apparent later.

Reliance on anaerobic glycolysis has another effect, the generation of NADH, or reducing equivalents. These reducing equivalents cannot be oxidized because there is insufficient O₂. One way for the cell to “dispose” of them is to use them in the synthesis of lipids. This may be one source of the liver lipids observed in non-alcoholic steatohepatitis. Just as the metabolism of alcohol by the liver produces “excess” reducing equivalents which lead to fatty liver, so to may anaerobic glycolysis due to chronic diff-use hypoxia from capillary rarefaction.

When cells are hypoxic, or when they alternate between oxic and hypoxic states, the production of superoxide is increased. This superoxide further decreases NO levels because NO and superoxide react with diffusion limited kinetics, and will exacerbate any effects of low NO. This may be what brings on the NO crisis and the constricted capillaries of Raynaud's phenomena. When capillaries become rarefacted, the tissue is especially sensitive to any hypoxic insult, to any change that decreases the perfusion of the volume of tissue, such as cold. When this happens, the tissue becomes hypoxic, superoxide is produced, NO is destroyed, capillaries become more constricted due to reduced vasodilatation which leads to further hypoxia, further superoxide and further constriction. The hypoxia exacerbates the low NO and vice versa. It is a case of positive feedback. One solution is to stop the capillary rarefaction in the first place. When NO is destroyed with superoxide, peroxynitrite is formed. Peroxynitrite is a strong oxidant which affects a number of enzymes. An enzyme that is affected is eNOS. Goligorsky et al. have reported that eNOS synthesizes NO from L-arginine, O₂, NADPH, and tetrahydrobiopterin. (Goligorsky et al., Relationships between caveolae and eNOS: everything in proximity and the proximity of everything, Am J Physiol Renal Physiol 283: F1-F10, 2002.)

Electrons are shuttled from NADPH, through calmodulin and onto the eNOS dimer. When the eNOS dimer is exposed to peroxynitrite, the zinc thiolate complex is destabilized, and eNOS becomes “uncoupled”. Zou et al. have shown it produces superoxide instead of NO. ( Zou et al., Oxidation of the zinc-thiolate complex and uncoupling of endothelial nitric oxide synthase by peroxynitrite, J. Clin. Invest. 109:817-826 (2002).)

In another aspect of the invention it, is appreciated that peroxynitrite injury may not be a case of too much NO, but may be a case of too little. Many of the experimental results showing increased damage due to increased NO, may be artifacts of the experimental techniques used. Most NO donors used in such experiments release NO indiscriminately. It is not surprising that releasing a compound as reactive as NO indiscriminately causes problems. Similarly, many of the NOS inhibitors not only inhibit NO production, they also inhibit superoxide production by NOS. Thus a “protective” effect of a NOS inhibitor on ischemic injury, doesn't necessarily demonstrate that the injury is a result of NO.

Even if only one cell becomes hypoxic, around that cell the resulting superoxide will destroy NO and the cell and cells in the vicinity will become further depleted in NO. With less NO, the signals of HIF-1α and VEGF will be attenuated, and capillary rarefaction may progress.

Reliance solely on O₂ levels for control of capillary spacing would be problematic in tissues where O₂ levels do not reflect capillary spacing, such as in the gas exchange regions of the lung.

Cancer

According to another embodiment of the invention, it is appreciated that the presence of NO during hypoxia may prevent cells from dividing while under hypoxic stress, when cells are at greater risk for errors in copying DNA. One cell function is the regulation of the cell cycle. This is the regulatory program which controls how and when the cell replicates DNA, assembles it into duplicate chromosomes, and divides. The regulation of the cell cycle is extremely complex, and is not fully understood. However, it is known that there are many points along the path of the cell cycle where the cycle can be arrested and division halted until conditions for doing so have improved. The p53 tumor suppressor protein is a key protein in the regulation of the cell cycle, and it serves to initiate both cell arrest and apoptosis from diverse cell stress signals including DNA damage and p53 is mutated in over half of human cancers as reported by Ashcroft et al. in “Stress Signals Utilize Multiple Pathways To Stabilize p53” (Molecular And Cellular Biology, May 2000, p. 3224-3233.). Hypoxia does initiate accumulation of p53, and while hypoxia is important in regulating the cell cycle, hypoxia alone fails to induce the down stream expression of p53 mRNA effector proteins and so fails to cause arrest of the cell cycle. Goda et al. have reported that Hypoxic induction of cell arrest requires hypoxia-inducing factor-1 (HIF-1α). (Hypoxia-Inducible Factor la Is Essential for Cell Cycle Arrest during Hypoxia. Molecular And Cellular Biology, January 2003, p. 359-369.) Britta et al. have reported that NO is one of the main stimuli for HIF-1α. (Britta et al., Accumulation of HIF-1α under the influence of nitric oxide, Blood, Feb. 15, 2001, Volume 97, Number 4.) In contrast, NO does cause the accumulation of transcriptionally active p53 and does cause arrest of the cell cycle and does cause apoptosis. (Wang et al., P53 Activation By Nitric Oxide Involves Down-Regulation Of Mdm2, The Journal Of Biological Chemistry Vol. 277, No. 18, Issue Of May 3, Pp. 15697-15702, 2002.)

Hypoxia in tumors during cell division increases genetic instability, including increased mutations, deletions and transversions. Graeber et al. disclose that Hypoxia in tumors selects for tumor cells that are resistant to hypoxia mediated apoptosis. (Graeber et al., Hypoxia-mediated selection of cells with diminished apoptotic potential in solid tumours, Nature, Jan. 4, 1996;379(6560):88-91.) If an error is introduced in the p53 gene (as has occurred in more than half of all cancers) then that cell (and all daughter cells) no longer has one of the main tumor suppressor genes which prevent cancers from growing uncontrollably. Many tumor cells are quite resistant to hypoxia, hypoxia confers resistance to both chemotherapy drugs and radiation, and many tumors have hypoxic regions. Postovit et al. report that tumor invasiveness is increased by hypoxia, and that increase is blocked by compounds that release NO. Postovit et al., Oxygen-mediated Regulation of Tumor Cell Invasiveness Involvement Of A Nitric Oxide Signaling Pathway, The Journal Of Biological Chemistry, Vol. 277, No. 38, Issue of September 20, pp. 35730-35737, 2002.) Postovit et al. also note that the various NOS enzymes use O₂ to generate NO, and so will produce less NO under conditions of hypoxia, exactly the time when more NO is needed. Hypoxia induces the production of VEGF and so reduces apoptosis due to serum deprivation. There are many growth factors in serum, only some of which have been characterized. One wonders if the increase in insulin (which is also a growth factor for endothelial cells) in type 2 diabetes might be compensatory, to reduce apoptosis of the vasculature due to low basal NO levels. Marchesi et al. disclose that administering L-arginine to type 2 diabetics increases insulin sensitivity and increases forearm blood flow. (Marchesi et al., Long-Term Oral L-Arginine Administration Inproves Peripheral and Hepatic Insulin Sensitivity in Type 2 Diabetic Patients, Diabetes Care 24:875-880, 2001.) This indicates that reduced basal NO levels are characteristic of type 2 diabetes. It is further reported by Wideroff et al. that the total incidence of cancer, as well as cancers of the breast, liver, kidney, pancreas, colon, brain, and others are all elevated in patients diagnosed with diabetes. (Wideroff et al., Cancer Incidence in a Population-Based Cohort of Patients Hospitalized With Diabetes Mellitus in Denmark, J Natl Cancer Inst 1997; 89:1360-5.)

In another aspect of the invention, it is appreciated that early menarche and increased height are markers for increased basal metabolism due to low basal NO. In breast cancer, it is well known that factors that increase risk are early menarche, never being pregnant, never breast feeding, living in a developed region, living in an urban area, being tall. For example, Yoo et al. have reported that the age-corrected incidence for ethnic Chinese living in Los Angeles is 48.7 per 100,000 while for Chinese living in Shanghai it is 21.2; for ethnic Japanese in L. A. it is 72.2, in Osaka it is 21.9), (Epidemiology of breast cancer in Korea: Occurrence, high-risk groups, and prevention, J Korean Med Sci 2002; 17: 1-6.). Factors that do not seem to affect incidence of breast cancer include PCB or DDT exposure suggesting that exposure to “chemicals” is not the main factor. It may be that it is the vascular proliferation and increased capillary density that accompanies pregnancy and lactation that provides the protective effects. It has been suggested that the increased exposure to estrogen “hormones” which accompanies early menarche is causal. However, while many breast tumors are estrogen dependant, it is not clear how estrogen would induce the genetic abnormalities that lead to cancer initiation. Pregnancy induces many growth factors, it would seem unlikely that the many growth factors of pregnancy are some how “protective”, but the few growth factors of early menarche are “causal”. The urban/rural and developed/undeveloped effects may be due to AAOB and their effect on basal NO levels. Many of the known protective factors are consistent with greater capillary density and many of the known risk factors are consistent with decreased capillary density. That the incidence of breast cancer in the developed world is in places more than twice that of the undeveloped World implies that most developed World cancers are caused by the environmental changes accompanying development.

Migration studies have shown that the breast cancer incidence of migrants initially matches that of location of origin, and over time shifts to match that of the area migrated to. However, Grover et al. have shown that the time constant for this shift is on the order of decades (Commentary The initiation of breast and prostate cancer, Carcinogenesis vol. 23 no. 7 pp. 1095-1102, 2002.). It has been shown that exposure to antibiotics increases the risk of breast cancer. (Velicer et al., Antibiotic use in relation to the risk of breast cancer. JAMA. 2004; 291: 827-835. ) Antibiotic exposure may modify breast cancer risk by eliminating AAOB resident on the skin, or perhaps even in the breast ducts.

Adverse Consequences of ATP Depletion

Since virtually all metabolic processes utilize ATP, insufficient ATP will compromise virtually all cellular functions. A reduction in ATP can lead to apoptosis, and if severe, to necrosis. Such apoptosis and necrosis would be expected at those cells farthest from a capillary and would likely occur one cell at a time. Diff-use apoptosis or necrosis would be difficult to observe, yet might explain the chronic diffuse inflammation also observed in many of these same degenerative diseases.

Any insults that increase metabolic load, would be expected to be exacerbated under conditions of ATP depletion due to nitropenia.

In all cells, damaged and misfolded proteins are disposed of by conjugation with ubiquitin and transport to the proteasome where they are disassembled by ATP mediated proteolysis. Under conditions of insufficient ATP, it would be expected that damaged and ubiquitinated proteins would accumulate to pathological levels, as is observed in many disorders. For example in Alzheimer's disease, amyloid deposits accumulate in the brain. Similarly, in Parkinson's disease, Lewy bodies composed of damaged hyperubiquitinated proteins accumulate in the brain. Similarly, in Rheumatoid arthritis, amyloid deposits in abdominal fat are not uncommon. Similarly, in patients undergoing dialysis, accumulation of amyloid is not uncommon. In congestive heart failure, damaged, hyperubiquitinated proteins accumulate in the heart. The pathological accumulation of proteins may be a symptom of insufficient ATP due to nitropenia.

In another aspect of the invention, it is appreciated that increased sodium intake may increase metabolic load on the kidney and increase sensitivity to ischemic insults, thereby accelerating the progression of low NO induced capillary rarefaction. Increased cell division while under hypoxic stress will lead to increased mutations and increase the likelihood of a cancerous transformation. It should be recognized that under conditions of chronic low NO, after capillaries have become rarefacted, the cells-farthest from the capillaries are always in a chronic state of hypoxic stress and so are especially sensitive to insults that drive them over the edge and into apoptosis or necrosis or genetic instability. Any insult that increases metabolic load will increase the local hypoxia and increase the rate at which they die or mutate. In the kidney, a major metabolic load is due to sodium resorption. Increased sodium will increase metabolic load on the kidney and increase sensitivity to ischemic insults and accelerate the progression of low NO induced capillary rarefaction. This may explain why a high salt diet exacerbates hypertension and kidney damage. Lieber et al. have reported that in the liver, alcohol metabolism can displace up to 90% of other metabolic substrates. (Lieber et al., Pharmacology and Metabolism of Alcohol, Including Its Metabolic Effects and Interactions With Other Drugs, Clinics in Dermatology 1999;17:365-379.) Stressing cells in the liver with alcohol would be expected to worsen their response to hypoxic stress. Hypertrophic hearts are especially vulnerable to hypoxia. Thus many of the recognized risk factors for degenerative diseases are factors that may be well tolerated in patients with normal capillary density, but may exacerbate the metabolic deficiency of any tissue with refracted capillaries.

Similarly, mitochondria depletion will also increase vulnerability to ischemic or hypoxic insults.

In another aspect of the invention, it is appreciated that preventing the necrotic death of cells by preventing the capillary rarefaction and mitochondria depletion that leads to their hypoxic/ischemic death may prevent autoimmune disorders. When cells are exposed to chronic hypoxia, the production of reactive oxygen species (ROS) is increased, and there is increased damage to the cells metabolic machinery and ultimately to the cells DNA. Decreased metabolic capacity will decrease capacity for repair of damage due to ROS and due to exogenous carcinogen exposure. Over time, the damage accumulates and will ultimately result in one of 3 events. The cell will undergo deletion of cancer preventing genes and the cell will become cancerous, the cell will die through necrosis, or the cell will die through apoptosis. When cells die, either through necrosis or apoptosis, the cell debris must be cleared from the site. Dead cells are phagocytosed by immune cells, usually dendritic cells. When dendritic cells phagocytose a body, it is digested by various proteolytic enzymes into antigenic fragments, and then these antigens are attached to the major histocompatability complex (MCH1, MHC2) and the antigen-MHC complex is moved to the surface of the cell where it can interact with T cells and activate the T cells in various ways. Any cell injury releases adjuvarts which stimulate the immune system in various ways. In general, cells that undergo necrosis stimulate a greater immune response than cells that undergo apoptosis. Chronic exposure of dendritic cells to dead and dying cells is therefore likely to lead to autoimmune disorders. Chronic inflammation is well known to increase cancer incidence.

According to another aspect, it is appreciated that the generalized shrinkage of organs that occurs with age may result from the gradual apoptotic loss of cells due to capillary rarefaction/mitochondria depletion. When cells die through necrosis, they induce inflammation and the cell debris must be phagocytosed for disposal. When necrotic tissue is phagocytosed by dendritic, cells the dendritic cells mature and express antigens derived from the necrotic tissue and the major histocompatability complex resulting in the induction of immunostimulatory CD4+ and CD8+ T cells. Significant quantities of necrotic tissue (one cell at a time) could very well prime the immune system for autoimmune diseases. It should be recognized that a significant component of inflammation is increased production of superoxide. This superoxide will destroy NO and locally exacerbate nitropenia.

Any organ that experiences capillary rarefaction/mitochondria depletion is a candidate for autoimmune sensitization. The progression from PRP to SSc and autoimmune sensitization is simply a reflection of greater capillary rarefaction and increased opportunities for autoimmune sensitization. Similarly, other autoimmune disorders are due to chronic inflammation induced by capillary rarefaction.

Bukhari et al. have demonstrated that in primary Raynaud's phenomena (PRP), the nailfold capillaries are sparser (slightly) than in normal controls, and more abundant than in patients that have progressed to systemic sclerosis (SSc). (Bukhari et al., Increased Nailfold Capillary Dimensions In Primary RaynaudIS Phenomenon And Systemic Sclerosis, British Journal Of Rheumatology Vol 24 NO 35: 1127-1131, 1996.)

They found that the capillary density decreased from 35 loops/mm2 (normal controls) to 33 (PRP), to 17 (SSc). The average distance between capillary limbs was 18μ, 18μ, and 30μ for controls, PRP and SSc. Even if only a few cells between each capillary were damaged due to hypoxia at any one time, that damage would accumulate, and eventually, those cells would necrose and be phagocytosed. With so many opportunities for autoimmune sensitization, it would seem only a matter of time before autoimmune sensitization occurred. If the stressed cells are removed through apoptosis, there might be no sign on autopsy that they were ever there. The generalized shrinkage of organs that occurs with age might result from the gradual apoptotic loss of cells due to capillary rarefaction.

In another aspect of the invention, it is appreciated that low basal NO leads to fibrotic hypertrophy. Once a dead cell has been cleared, a new cell cannot easily take its place, because there is insufficient O₂ to support it. Any such new cell would suffer the same fate. The space can remain empty, in which case the organ shrinks, the capillaries draw closer together, new cells are now deprived of the VEGF formally produced by the now missing cell, so capillaries ablate and the hypoxic zone reforms. This could result in a general shrinkage of the affected tissues. In tissues that support fibrosis, relatively inert collagen fibers can fill the space. Since the metabolic requirements of the body for the particular organ in question are not reduced, the organ may attempt to grow larger, but now with a significant fibrous content. This may result in fibrotic hypertrophy, such as of the heart, liver and kidney. Some organs, such as the brain, cannot grow larger or smaller because the 3 dimensional connectivity of nerves and blood vessels are important, and cannot be continuously and simultaneously mapped onto an asymmetrically shrinking brain. The space must be filled with something, and β-amyloid might be the (not so inert) space filler. The kidney cannot grow larger because of the renal capsule, so the number of living cells becomes smaller and they are replaced with fibrotic tissue. If the dead cells are cleared, the tissue shrinks, and the ratio of NO/O₂ goes down again, and the capillaries again become sparser. This may set up the vicious circle of end stage renal disease, congestive heart failure/cardiac hypertrophy, primary biliary cirrhosis, Alzheimer's disease, atherosclerosis, inflammatory bowel disease, hypertrophic scar formation, and the multiple connective tissue diseases starting with Raynaud's phenomena and ending with Systemic Sclerosis and primary Sjogren's syndrome where capillary rarefaction is also observed. Ferrini et al, have shown that a reduction in basal NO levels through chronic inhibition of NOS with L-NAME leads to generalized fibrosis of the heart and kidneys. (Ferrini et al., Antifibrotic Role of Inducible Nitric Oxide Synthase. Nitirc Oxide: Biology and Chemistry Vol. 6, No. 3, pp. 283-294 (2002).) It may be that low basal NO leads to fibrotic hypertrophy.

Capillary density and mitochondria depletion as factors in appetite regulation

In another embodiment of the invention, it is appreciated that capillary rarefaction/mitochondria depletion affects a subject's ability to control their appetite. Capillary rarefaction is observed in the brains of aged humans and animals. Capillary rarefaction is associated with declines in circulating growth factors including insulin like growth factor-1. Neurogenesis in the adult brain is coordinated with angiogenesis. Since the brain regulates many homeostatic functions, increased diffusion lengths between capillaries to control elements of the brain might be “interpreted” as inadequate blood concentrations of those species. The flux of glucose in the brain is quite close to normal metabolic needs, where maximum glucose flux is only 50 to 75% greater than glucose consumption and the glucose transporters across the blood brain barrier are saturable, steriospecific and independent of energy or ion gradients. A large part of the regulation of appetite is mediated through the brain, and capillary rarefaction may cause an adequate blood concentration of “nutrients” (or marker compounds proportional to “nutrients”) to be interpreted as insufficient. This may be one cause of the epidemic of obesity. Individuals who cannot control their appetite might simply have too long a path between their capillaries and the brain cells that trigger appetite. Their brains might be telling them they are “starving”, because those brain cells that are a little bit too far from a capillary are “starving”. This may not result simply from the longer diffusion path, but by consumption of the “nutrient” by the intervening cells. When cells are hypoxic or have insufficient mitochondria, and are unable to derive ATP from oxidative glycolysis, they instead generate ATP through anaerobic glycolysis. The amounts of glucose required to support metabolism through anaerobic glycolysis is 19 times greater than through oxidative glycolysis. Thus a single hypoxic/mitochondria depleted cell could consume as much glucose as 19 non-hypoxic cells. If even a few partially hypoxic cells were between a “glucose sensing cell” and the capillary which is the glucose source, the “glucose sensing cell” would necessarily receive an erroneously low reading. While neurons generate ATP only through oxidative phosphorylation, other brain cells such as astrocytes can also generate ATP through anaerobic glycolysis. A few hypoxic astrocytes in proximity to a neuron would likely deprive that neuron of glucose. The craving for sugar and carbohydrate that plague many people may derive from specific neurons being deprived of glucose due to nearby hypoxic astrocytes. The elevated blood sugar may be an attempt to get more glucose to those cells, but because the glucose transporters are saturable and the pathway is blocked by too many hypoxic astrocytes, it may not be possible for blood sugar to be high enough. The association of obesity with chronic degenerative diseases may not be because obesity “causes” them, but because the thing that does cause obesity (capillary rarefaction and mitochondria depletion) also causes degenerative diseases. Kingwell has shown that exercise does increase basal NO levels in normal healthy and hypercholesterolemic individuals. (Kingwell, Nitric oxide-mediated metabolic regulation during exercise: effects of training in health and cardiovascular disease. FASEB J. 14, 1685-1696 (2000).) It may be the positive effects of exercise on obesity could be mediated through nitric oxide mediated angiogenesis. Induction of ketosis, either through starvation or through a ketogenic diet (low carbohydrate) causes the liver to generate ketone bodies acetoacetate and β-hydroxybutyrate from lipids. These ketone bodies circulate and are used by neurons instead of glucose in oxidative phosphorylation. A ketogenic diet increases the threshold for seizure induction through electroshock, hyperbaric O₂, and chemically induced seizures. A ketogenic diet has been used to treat epilepsy for over half a century. It has been suggested that the anti-seizure effects of a ketogenic diet are due to greater neuron energy reserves. The appetite suppression effects of a ketogenic diet may similarly derive from greater neuron energy reserves.

The inventor has applied AAOB over a year and has noticed a pronounced reduction in appetite, and has lost ˜30 pounds over the course of a year, simply by eating less without pronounced discomfort. While the inventor was formally unable to function while skipping meals, he is now able to skip multiple meals with no loss in ability to function either mentally or physically.

Capillary Rarefaction/Mitochondria Depletion as a Cause of Non-Insulin Dependent Diabetes

According to another aspect of the invention, it is appreciated that capillary rarefaction/mitochondria depletion may be a cause of non-insulin dependent diabetes. Non-insulin dependent diabetes (NIDDM) is also known as the Metabolic Syndrome or Diabetes type 2, and is characterized by insulin resistance. The sensitivity of the body to insulin is reduced, and insulin levels increase. The “cause” remains unknown in spite of intense research. It is observed in all developed regions of the World, across many cultures and many ethnic groups. People with NIDDM have high blood glucose, high blood triglycerides, are typically obese, hypertensive, and typically have significant visceral fat.

Other symptoms accompany NIDDM, which the inventor believes point to capillary rarefaction as the cause. In a study of 40 men, with and without NIDDM, obese (BMI 29) and lean (BMI 24) (10 of each), Konrad et al. report that blood lactate levels at rest were 1.78, 2.26, 2.42, and 2.76 (mM/L) for lean men without, obese men without, lean men with NIDDM, obese men with NIDDM respectively. (Konrad et al., A-Lipoic acid treatment decreases serum lactate and pyruvate concentrations and improves glucose effectiveness in lean and obese patients with type 2 diabetes, Diabetes Care 22:280-287, 1999.) Lactate is a measure of anaerobic glycolysis. When O₂ is insufficient to generate ATP through oxidative phosphorylation, cells can produce ATP through anaerobic glycolysis. One of the products of anaerobic glycolysis is lactate, which must be exported from the cells, otherwise the pH drops and function is compromised. Blood lactate is commonly measured in exercise studies, where an increase indicates the work load at which maximum oxidative work can be done. Higher levels of lactate at rest would indicate increased anaerobic glycolysis at rest, which is consistent with capillary rarefaction. It is interesting to note that lean diabetic men had higher lactate than obese non-diabetic men.

Muscle cells of NIDDM individuals have higher ratios of glycolytic to oxidative enzymes than do non-NIDDM individuals. NIDDM individuals thus derive a greater fraction of their muscle energy from anaerobic glycolysis than from oxidative phosphorylation.

Measurement of muscle pH and phosphate species with MRI before and during muscle activity has demonstrated that men with well controlled diabetes type 1 have altered muscle physiology. In a study by Crowther et al., Diabetic men have reduced oxidative capacity, and derive a greater fraction of their ATP from anaerobic glycolysis, and this difference is apparent even at rest. (Crowther et al., Altered energetic properties in skeletal muscle of men with well-controlled insulin-dependent (type 1) diabetes, Am J Physiol Endrocrinol Metab 284: E655-E662, 2003.) This study is interesting because it measures lactate production in vivo through pH changes. In their study they noted that some individuals had two distinct populations of muscle cells with different pH and hence lactate production, 4 of 10 diabetics and 2 of 10 non-diabetics. In their study they simply averaged the values, however, distinct populations of cells with different lactate production is indicative of different oxidative phosphorylation capacity and hence different O₂ supply.

Woman with NIDDM have decreased VO₂max when compared with both lean and obese controls. This reduced VO₂max is apparent even in the absence of any cardiovascular complications. Women with NIDDM had lower peak work production and greater blood lactate levels, both at rest and during exercise.

These observations of increased anaerobic glycolysis in people with both type 1 and type 2 diabetes are consistent with chronic decreased O₂ delivery to the peripheral tissues, and/or to insufficient mitochondria. That this increased anaerobic glycolysis is observed at rest, when metabolic demand is at a minimum, indicates that this decreased O₂ delivery/insufficient mitochondria is chronic.

Capillary Rarefaction/Mitochondria Depletion as a Cause of Insulin Dependent Diabetes (Diabetes Type 1).

Diabetes type 1 is characterized by the autoimmune destruction of the pancreatic islets that release insulin in response to increases in blood glucose levels. ATP depletion due to nitropenia mediated through capillary rarefaction, mitochondria depletion, and reduced expression of glycolytic enzymes will push the mitochondria in the pancreas to higher potential, which will generate superoxide, which will lead to induction of uncoupling protein, which will then cause ATP levels to fall, and which will then lead to islet apoptosis or necrosis. Autoimmune sensitization can then occur. Once the immune system is sensitized to attack the pancreatic islets, superoxide is produced in their vicinity, which lowers local NO levels still further, exacerbating capillary rarefaction, mitochondria depletion, and insufficient glycolytic enzymes.

Treatment of Liver Inflammation with AAOB

Primary biliary cirrhosis is associated with Raynaud's phenomena, pruritus, sicca syndrome, osteoporosis, portal hypertension, neuropathy, and pancreatic insufficiency. Liver abnormalities are associated with rheumatic diseases. Elevated liver enzymes are a symptom of liver inflammation, and elevated liver enzymes are observed as an early symptom of “asymptomatic” primary biliary cirrhosis.

Elevated liver enzymes are commonly seen in patients with collagen diseases, including biliary cirrhosis, autoimmune hepatitis and nodular regenerative hyperplasia of the liver matoid arthritis (RA), polymyositis and dermatomyositis (PM and DM), systemic sclerosis (SSc), mixed connective tissue disease (MCTD) and polyarteritis nodosa (PAN).

The progression of primary biliary cirrhosis is characterized by 4 stages, first is the inflammatory destruction of the intrahepatic small bile ducts due to previously unknown causes, followed by the proliferation of ductules and/or piecemeal necrosis, followed by fibrosis and/or bridging necrosis, followed by cirrhosis. Benvegnù et al. report a correlation between cirrhosis of the liver and liver cancer. (Benvegnù et al., Evidence for an association between the aetiology of cirrhosis and pattern of hepatocellular carcinoma development. Gut 2001;48:110-115.) A variety of autoimmune connective tissue diseases are associated with primary biliary cirrhosis, including Sjogren's syndrome, scleroderma, CREST syndrome (calcinosis, Raynaud's phenomenon, esophageal dysmotility, sclerodactyly, or telangiectasia), inflammatory arthritis, or thyroid disease.

The treatment of choice for primary biliary cirrhosis is oral ursodeoxycholic acid. This is a hydrophilic bile salt that displaces other more toxic hydrophobic bile salts in the hepatic circulation. While the mechanism is not fully understood, a component of the therapeutic effects may derive from reduced metabolic load on the liver through reduced bile synthesis.

While anti-mitochondrial anti-bodies are usually present in primary biliary cirrhosis, 5-10% of patients with PBC do not have such antibodies moreover, most of these patients have autoimmune antibodies to smooth muscle or nuclear factors. However, immunosuppressant therapy is not as effective at slowing the progression of PBC as oral ursodeoxycholic acid is. This indicates that autoimmune antibodies are not the cause of PBC, but instead are a consequence of some other cause.

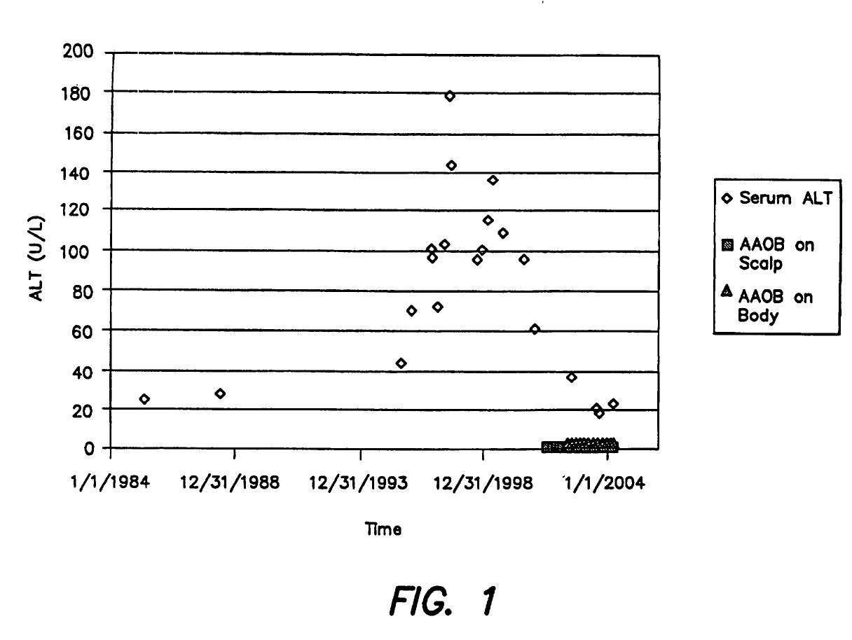

In one embodiment of the invention, application of AAOB to the scalp and body of an individual resulted in a lowering of liver enzymes. FIG. 1 shows a plot of liver enymes, alanine transaminase levels (SGPT or ALT) for a single individual both before and during application of AAOB to the scalp and body. Following application of the AAOB, the SGPT level dropped to the lowest point in nearly 20 years. Schoen et al. have reported that nitric oxide is known to trigger the initiation of liver regeneration. ( Schoen et al., Shear Stress-Induced Nitric Oxide Release Triggers the Liver Regeneration Cascade, Nitric Oxide: Biology and Chemistry Vol. 5, No. 5, pp. 453-464 (2001).) Thus the application of AAOB is shown to be effective in reducing elevated liver enzymes and the chronic liver inflammation that elevated liver enzymes indicate. While there is only sparse data to indicate the time scale of the reduction in liver enzymes following application of AAOB, it appears to not be instantaneous. A gradual reduction is consistent with the gradual resolution of long standing capillary rarefaction through capillary remodeling following increased basal NO levels.

Reducing liver inflammation slows the progression of PBC and of other liver diseases and reduces the progression to cirrhosis which is associated with liver cancer.

In another aspect of the invention, it is appreciated that “hypoxia” used to regulate capillary density may occur during sleep. Though not being bound by one particular theory, the drop in blood pressure and in blood flow rate that normally occurs during sleep is one of the body's normal “housekeeping” functions, and serves to reset the O₂ diffusion resistance between the capillaries and the cells that those capillaries support. According to Zoccoli et al., the normal drop in blood pressure at night is attributed to increased NO, where inhibition of NOS with L-NNA abolishes wake-sleep differences in cerebral blood flow. (Zoccoli et al., Nitric oxide inhibition abolishes sleep-wake differences in cerebral circulation, Am J Physiol Heart Circ Physiol 280: H2598-H2606, 2001.) Kapfis et al. have shown that inhibition of NOS in rats inhibits normal sleep. (Kapfis et al., Inhibition of nitric oxide synthesis inhibits rat sleep. Brain Research 664 (1994) 189-196. ) Weitzberg et al. have reported that humming greatly increases nasal NO by increase gas exchange with the sinuses where NO is produced. (Weitzberg et al., Humming Greatly Increases Nasal Nitric Oxide, Am J Respir Crit Care Med Vol 166. pp 144-145, 2002.) A number of the disorders associated with capillary rarefaction are also associated with disordered breathing at night, either snoring or sleep apnea. Obesity, age, cardiovascular disease, hypertension, rheumatoid arthritis, are all associated with disordered breathing during sleep. Therefore, it is appreciated that high levels of NO may be advantageous during sleep, and sweating at night as well as snoring may both physiological mechanisms to increase basal NO. High levels of NO during sleep increase the NO/O₂ ratio and so increase the “hypoxia” signal.

The hypothesis that capillary spacing is determined during sleep is supported by the exercise training philosophy of “living high-training low,” where athletes train at low altitude, but go to high altitude to live and sleep. Training at low altitude allows greater metabolic load on the muscles being trained, where hypoxia is induced by near maximal metabolic load. Inducing hypoxia by reducing O₂ supply at night might not be effective for muscle because of their high capacity for anaerobic respiration and high levels of O₂ storing myoglobin. However, avoiding subjecting muscle to nightly hypoxia with insufficient NO might be an explanation for why cancers of muscle are rare. Hypoxia in organs not under conscious control cannot be induced voluntarily through exercise. For example, erythropoietin is produced by the kidney under conditions of “hypoxia” and regulates the production of erythrocytes and Hct. Ge et al. have shown that Erythropoietin is up regulated almost immediately with hypobaric hypoxia with nearly a 50% increase after 6 hours at 2800 meters. (Ge et al., Determinants of erythropoietin release in response to short-term hypobaric hypoxia. J Appl Physiol 92: 2361-2367, 2002.) EPO is commonly given to kidney dialysis patients to compensate for the loss of EPO from diseased or missing kidneys and to raise hematocrit. However, raising hematocrit close to the “normal” range increases mortality over lower levels. In a randomized study of 1233 patients by Besarab et al., raising Hct to 42% resulted in a 22% greater death rate over 29 months than patients with Hct raised to 30% (183 vs. 150 deaths) and the causes of death were similar in the two groups, and characteristic of dialysis patients, there were simply more deaths in the high Hct group. (Besarab et al., The Effects Of Normal As Compared With Low Hematocrit Values In Patients With Cardiac Disease Who Are Receiving Hemodialysis And Epoetin, N Engl J Med 1998;339:584-90.) It may be that the elevated Hct decreased the basal NO level, and the increased death rate was due to decreased basal NO. The causes of death were similar because both groups actually have low NO levels, it is low NO that brought about the kidney damage in the first place. While low Hct is “bad”, low NO is bad too. Without a good way to increase basal NO levels (until now), balancing the increased O₂ capacity of the blood with the decreased NO concentration is a difficult treatment choice.

Alzheimer's Disease

Torre et al have reported that Alzheimer's disease (AD) is a microvascular disorder with neurological degeneration secondary to hypoperfusion, resulting in part from insufficient nitric oxide. (Review: Evidence that Alzheimer's disease is a microvascular disorder: the role of constitutive nitric oxide, Brain Research Reviews 34(2000) 119-136.)

AD does not occur in all individuals, and it does not occur in single or even a few episodes of hypoperfusion, rather it occurs over time, sometimes over many years. The course of Alzheimer's, while inexorable and monotonic, is not steady, and is not associated with known episodes of hypoperfusion or syncope. In the early stages there can be considerable variability in degree of neuropathy and in rate of decline. That is one factor that can make the diagnosis of Alzheimer's difficult in the early stages.

Levels of ischemia sufficient to produce the levels of oxidative damage observed in AD due to hypoperfusion would produce noticeable contemporaneous mental effects. Levels of hypoxia and ischemia not producing oxidative damage are noticeable. Levels of hypoperfusion resulting in confusion or syncope are typically not reported by Alzheimer's patients, so the oxidative damage must have occurred during a non-reportable time, it may have occurred during sleep.

During sleep, the metabolism of all parts of the body is reduced. The blood pressure falls and the blood flow decreases. The velocity of blood flow throughout the body decreases, and with less shear at the vessel walls eNOS is down regulated and NO production by eNOS is reduced. The energy demands of the brain are reduced. The brain however is still quite active and still requires substantial blood flow.

Hypothermia is known to reduce cerebral damage during ischemic events. Hypothermia both during and even after such events reduces brain damage by reducing the reperfusion injury. Sleep normally causes a drop in body temperature of 0.5-0.7° C. Mild hypothermia during sleep would independently reduce energy needs of the brain and would reduce the ischemic threshold for damage. The basal metabolism rises approximately 14% for every 1° C. of fever, so the “normal” reduction, during sleep, of 0.5-0.7° C. is a reduction of 7 to 10% in metabolic rate.

NO is known to be necessary in the reduction of basal temperature due to hypoxia. Almeida et al. have reported that when NO synthesis is inhibited with N-nitro-L arginine (L-NNA) the reduction in basal temperature following hypoxia is greatly diminished. (Almeida et al., Role of nitric oxide in hypoxia inhibition of fever, J. Appl Physiol. 87(6): 2186-2190, 1999.)

The reports of a “protective effect” on Alzheimer's associated with non-steroidal anti-inflammatory drugs (NSAIDs), could, in part, result from their effect in lowering body temperature.

The epidemiology of Alzheimer's is well studied in developed countries but much less so in underdeveloped countries. Reliable and consistent differential diagnosis across many patients, many physicians, and many cultures is difficult and perhaps fraught with error. That said, according to the present theory that the causal events of hypoxia occur during sleep, then the incidence should increase with increasing sleeping temperatures. Tables 1 and 2 show the incidence of Alzheimer's reported in a review article by Suh and Shah. (Guk-Hee Suh, Ajit Shah, Review Article: A review of the epidemiological transition in dementia—cross-national comparisons of the indices related to Alzheimer's disease and vascular dementia, Acta Pyschiatr Scand 2001: 104: 4-11.)

The temperatures were taken from tabulated monthly averages fromYahoo weather, www.yahoo.com. When data for the study city was unavailable, a nearby city was used (in parentheses).

The data was divided into two sets, a “developed” and an “undeveloped” group. Beijing was included in both, with 1987 data as “undeveloped” and 1999 data as “developed”. The two groups were divided on the basis of perceived per capita water consumption for bathing. The relevant population is the populations at risk for AD, the elderly. That population is likely to lag behind others in the adoption of new bathing practices.

Table 1 shows maximum and minimum average monthly temperatures and incidence of Alzheimer's Disease and Total Dementia for undeveloped cities. Table 2 shows maximum and minimum average monthly temperatures and incidence of Alzheimer's Disease and Total Dementia for developed cities.

TABLE 1

| | | | Average | Average | Prevalence | Prevalence |
|---|---|---|---|---|---|---|
| Undeveloped | Date of | Hottest | High | Low | Alzheimer's | Total |
| City | Study | month | Temperature | Temperature | Disease | Dementia |
| Beijing | 1987 | July | 87.4 | 70.9 | 0.4 | 0.8 |
| Shanghai | 1990 | July | 88.9 | 76.6 | 3 | 4.6 |
| Hong Kong | 1998 | July | 92.7 | 74.5 | 4 | 6.1 |
| Taiwan | 1998 | July | 90 | 77.9 | 2.3 | 4 |
| (Taipei) | | | | | | |
| Ibadan | 1997 | February | 91.8 | 75.4 | 1.1 | 1.4 |
| (Lagos) | | | | | | |
| Kerala | 1998 | April | 93.6 | 71.2 | 1.4 | 3.4 |
| (Bangalore) | | | | | | |
| Tokyo | 1982 | August | 87.6 | 75.2 | 1.2 | 4.8 |
| Okinawa | 1995 | July | 88 | 79 | 3.1 | 6.7 |
| Hiroshima | 1999 | August | 87.6 | 74.5 | 2.9 | 7.2 |
| Aichi | 1986 | August | 90 | 74.3 | 2.4 | 5.8 |
| (Nagoya) | | | | | | |
| Wuhan | 1981 | July | 88.9 | 76.6 | 0.1 | 0.5 |
| (Wuhu) | | | | | | |

TABLE 2

| | | | | | Prevalence | Prevalence |
|---|---|---|---|---|---|---|
| Developed | Date of | Hottest | Average | Average | Alzheimer's | Total |
| City | study | month | High | Low | Disease | Dementia |
| Beijing | 1999 | July | 87.4 | 70.9 | 4.8 | 7.8 |
| Boston | 1989 | July | 81.8 | 65.1 | 8.7 | 10.3 |
| Odense | 1997 | August | 69.4 | 52.2 | 4.7 | 7.1 |
| London | 1990 | July | 71.1 | 52.3 | 3.1 | 4.7 |
| Stockholm | 1991 | July | 71.4 | 56.1 | 6 | 11.9 |
| Rotterdam | 1995 | July | 85.5 | 43.7 | 4.5 | 6.3 |
| (Amsterdam) | | | | | | |

The bathing practice believed to be important is the washing of the head and scalp with detergents which washes off the natural population of autotrophic ammonia oxidizing bacteria which produce nitric oxide for absorption into the scalp. In one aspect of the invention, not washing one's head is protective regarding AD, the populations likely show mixed behavior with different patterns of head washing. In developed cities with abundant shampoo products and clean hot water, washing one's head is common, and the population that washes their head less frequently than once per week is likely small. Washing one's head is common in the developed cities, and the population that washes their head less than once per week is likely small. In the undeveloped cities, there are likely still a considerable number that wash their head frequently enough to be essentially free from autotrophic bacteria. That part of the population may represent the majority of the AD cases in the undeveloped cities.

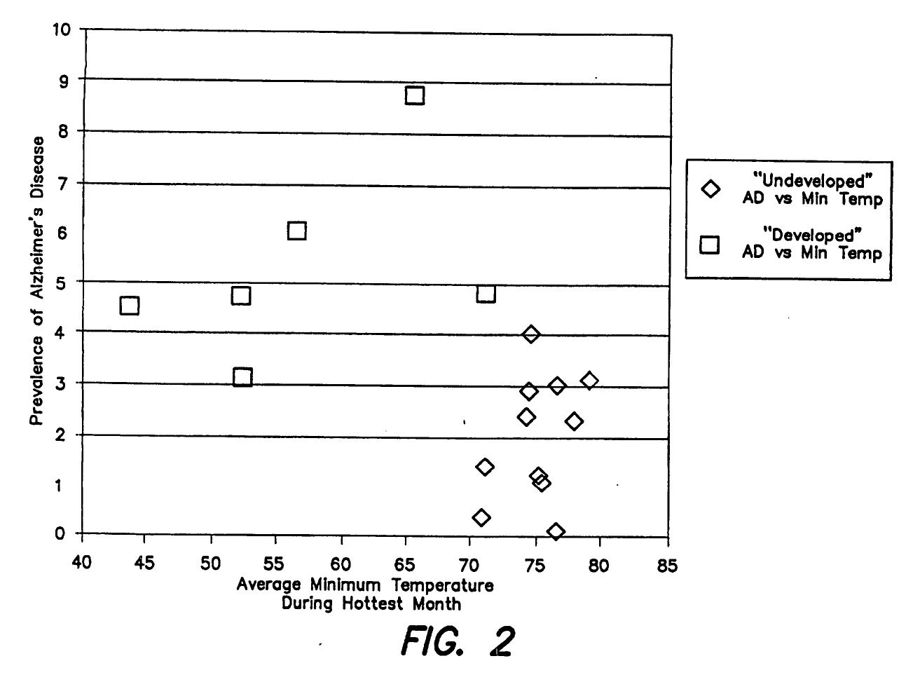

The data is plotted in FIG. 2, which shows the incidence of AD verses minimum temperature during the hottest month (i.e. temperature at night during sleep). The two data sets seem to fall into two groups, with increased minimum temperature correlating with increased incidence of AD, but with a different slope and intercept. The undeveloped intercept is around 70 F. Any intercept for the “developed” group would be off the chart, and would be unrealistic because heating would be used to raise the temperature into a “comfort zone”. While the progression of AD in undeveloped regions may show seasonality due to different sleeping temperatures, in developed regions, the intercept is below the minimum temperature that most people sleep at irrespective of outside temperature.

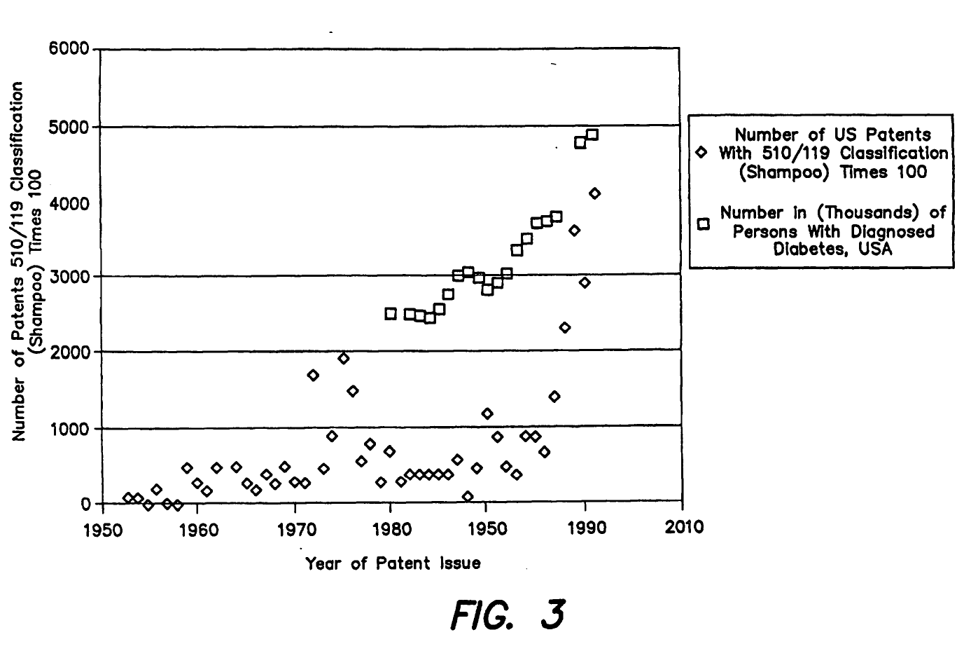

According to one aspect of the invention, it is appreciated that a factor in the current high incidence of AD is the improvement in shampoo technology that occurred in the early 1970's allowing one to shampoo often, even daily. Prior to that time, if one were to shampoo everyday, one's hair would “turn to straw”, and would be unaesthetic. It was the development of “conditioning” shampoos that allowed daily hair washing. A chart of the number of US patents issued on shampoo is shown in FIG. 3. There is a large surge in the early 1970's. Similarly, there is a surge in the number of persons diagnosed with diabetes type 1 approximately 10 to 15 years later. According to one aspect of the current invention, the current epidemic of obesity, diabetes, and AD derives from the development of conditioning shampoos and the adoption of their frequent use.

Other adverse health effects that are associated with hypertension may also be consequences of low basal NO. In hypertension, there is reduced vascular reactivity. The decreased response to vasodilatation is also consistent with low basal NO. NO is a diffusible molecule that diffuses from a source to a sensor site where it has the signaling effect. With low NO levels, every NO source must produce more NO to generate an equivalent NO signal of a certain intensity a certain distance away. NO diff-uses in 3 dimensions and the whole volume within that diffusion range must be raised to the level that will give the proper signal at the sensor location. This may result in higher NO levels at the source and between the source and the sensor. Adverse local effects of elevated NO near a source may then arise from too low a NO background. There is some evidence that this scenario actual occurs. In rat pancreatic islets, Henningsson et al have reported that inhibition of NOS with L-NAME increases total NO production through the induction of NOS. (Chronic blockade of NO synthase paradoxically increases islet NO production and modulates islet hormone release. Am J Physiol Endocrinol Metab 279: E95-E107, 2000.) Increasing NO by increasing NOS activity will only work up to some limit. When NOS is activated but is not supplied with sufficient tetrahydrobiopterin (BH4) or L-arginine, it becomes “uncoupled” and generates superoxide (O₂—) instead of NO. This O₂— may then destroy NO. Attempting to produce NO at a rate that exceeds the supply of BH4 or L-arginine may instead decrease NO levels. This may result in positive feedback where low NO levels are made worse by stimulation of NOS, and uncoupled NOS generates significant O₂— which causes local reactive O₂ species (ROS) damage such as is observed in atherosclerosis, end stage renal disease, Alzheimer's, and diabetes.

Osteoporosis

Osteoporosis is a disorder that affects many elderly. The age adjusted incidence of bone fractures in the elderly is increasing. The incidence of childhood distal forearm fractures has increased in the last 30 years, as reported by S. Khosla et. al. in Incidence of childhood distal forearm fractures over 30 years, in JAMA. 2003; 290;: 1479-1485. Nitric oxide is well known to affect bone density. Some of the positive effects of estrogen on bone density are mediated through the effect of estrogen on NO metabolism, where S. J. Wimalawansa reports that nitroglycerin is as effective as estrogen to prevent bone loss in “Nitroglycerin therapy is as efficacious as standard estrogen replacement therapy (Premarin) in prevention of oophorectomy-induced bone loss: a human pilot clinical study(Journal of Bone and mineral research Vol. 15, NO. 11, 2000.). It may be that the increase in fractures during childhood and in the elderly is a consequence of the loss NO from the loss of AAOB on the skin. Replacing the AAOB on the skin will reduce osteoporosis.

Aging

A gents to slow the progression of aging have been searched for since antiquity, but to little effect. The only demonstrated treatment that prolongs life is calorie restriction, where Holloszy reported that restricting food intake to 70% of ad lib controls, prolongs life in sedentary rats from 858 to 1,051 days, almost 25%. (Holloszy, Mortality rate and longevity of food restricted exercising male rats: a reevaluation. J. Appl. Physiol. 82(2): 399-403, 1997.) The link between calorie restriction and prolonged life is well established, however, the causal mechanism is not. Lopez-Torres et al. reported that the examination of liver mitochondrial enzymes in rats indicates a reduction in H₂O₂ production due to reduced complex I activity associated with calorie restriction. (Lopez-Torres et al., Influence Of Aging And Long-Term Caloric Restriction On Oxygen Radical Generation And Oxidative DNA Damage In Rat Liver Mitochondria, Free Radical Biology & Medicine Vol. 32 No 9 pp 882-8899, 2002.) H₂O₂ is produced by dismutation of O₂—, which is a major ROS produced by the mitochondria during respiration. The main source of O₂— has been suggested by Kushareva et al. and others to be complex I which catalyzes the NAD/NADH redox couple by reverse flow of electrons from complex III, the site of succinate reduction. The free radical theory, proposed by Beckman, of aging postulates, that free radical damage to cellular DNA, antioxidant systems and DNA repair systems accumulates with age and when critical systems are damaged beyond repair, death ensues. (Beckman, The Free Radical Theory of Aging Matures. Physiol. Rev. 78: 547-581, 1998.) It is to be recognized that the mitochondria are the major producers of superoxide, and that the superoxide production rate and mitochondria efficiency depends strongly on the mitochondria potential. The lower the mitochondria potential, the more efficient is the production of ATP, and the lower is the production of superoxide. Calorie restriction may exert its protective effects on aging via forcing the cells to produce more mitochondria to achieve greater metabolic efficiency, a side effect of which is reduced superoxide.

In addition to free radical damage leading to senescence, there is also programmed senescence based on the length of telomeres which shorten with each cell division. NO has been demonstrated by Vasa et al. to activate telomerase and to delay senescence of endothelial cells. (Vasa et al., Nitric Oxide Activates Telomerase and Delays Endothelial Cell Senescence. Circ Res. 2000;87:540-542.) Low basal NO will increase basal metabolic rate by disinhibition of cytochrome oxidase. Increased basal metabolism will also increase cell turn-over and growth rate. Capillary rarefaction, by inducing chronic hypoxia may increase free radical damage and may also increase cell turn-over, and so accelerate aging by both mechanisms.

In another aspect of the invention, it is appreciated that AAOB affects the age of puberty onset. An interesting observation in human aging is that the age of menarche declines as a region becomes more developed. A number of factors have been used to explain this, however the correlation that “best” fits the data, is an inverse relationship with illiteracy rate proposed by Thomas et al. (Thomas et al., International Variability of Ages at Menarche and Menopause: Patterns and Main Determinants. Human Biology, April 2001, v. 73, no. 2, pp. 271-290.) However, Freedman et al. reported that in the US, the median ages of menarche in 1974 were 12.9 and 12.7 years for black and white girls respectively. (Freedman et al., Relation of Age at Menarche to Race, Time Period, and Anthropometric Dimensions: The Bogalusa Heart Study, Pediatrics 2002;110(4).) In 1994 they were 12.1 and 12.5 years. It has been suggested that this decline in age of menarche relates to dietary practices, in particular to increased fat in the diet. However, from 1965 to 1995, the percentage of fat in the diet of 11 -18 year olds actually dropped from 38.7% to 32.7%. In Norway, the age of menarche has dropped from 16.9 years in 1850 to 13.3 years in 1950. The change is quite linear over time. In the US, from 1910 to 1950, the drop was from 14 to 13, also quite linear, with no increase observed during the Depression, when presumably food availability would have been less. The age of puberty may be actually due to the loss of AAOB through bathing, and not due to increased availability of food. The association of early menarche with literacy rate may be due to the adoption of the Western notion that “cleanliness is next to godliness.” Disease is not associated with dirt, disease is associated with pathogens, which may or may not be associated with dirt. The elimination of diarrheal diseases due to modern sanitation may not be due to increased bathing, but may be due to sanitary disposal of pathogen containing fecal matter, and the prevention of the contamination of the water supply by pathogen containing wastes.

Life expectancy generally increases with economic development. This increase is due to a number of factors. Infant mortality decreases due to declining starvation, diarrheal diseases, and other infections. Life expectancy of adults increase due to better access to health care. However, some developed countries have started to see the life expectancy of their aged populations actually decline. In the Netherlands, the life expectancy at age 85 has declined in men since the 1980's and in both sexes since 1985/89 as reported by Nusselder et al. (Nusselder et al., Lack of improvement of life expectancy at advanced ages in The Netherlands, International Journal of Epidemiology 2000;29: 140-148. ) There are increases due to mental disorders (presumably Alzheimer's Disease), cancer and diabetes, and chronic obstruction pulmonary disease, all conditions expected to be exacerbated by a reduction in basal NO levels.

Allergies and Autoimmune Disorders

In another aspect of this invention, it is appreciated that autotrophic ammonia oxidizing bacteria may produce protective aspects for allergies and autoimmune disorders. The incidence of allergy among children has been increasing throughout the developed world and asthma is now the most common chronic disease of childhood. No clear explanation of the different incidence of allergies and asthma among different population groups has been proposed. The data is quite complex and seemingly contradictory. Autoimmune disorders are also common. The best known is perhaps Diabetes Type 1, which results from the destruction of the insulin producing cells in the pancreas by the immune system. Recurrent pregnancy loss is also associated with autoimmune disorders where the number of positive autoimmune antibodies correlated positively with numbers recurrent pregnancy losses. Systemic Sclerosis, Primary Biliary Cirrhosis, autoimmune hepatitis, and the various rheumatic disorders are other examples of autoimmune disorders.

In general, the incidence of allergies increases with affluence, both as the affluence of a population increases through development, and within a population the incidence is higher in the most affluent group. However, Platts-Mills et al. have reported that in the US, the incidence of asthma in urban African Americans is three times that of suburban children. (Platts-Mills et al., Asthma and Indoor Exposure to Allergens, New England Journal of Medicine Volume 336:1382-1384 May 8, 1997 Number 19.)

Rasmussen et al. have reported that Swedish conscripts born in Africa show lower allergy symptoms than those of African decent born in Sweden. (Rasmussen et al., Migration and atopic disorder in Swedish conscripts, Pediatr Allergy Immunol 1999: 10: 209±215.) This paper shows significant differences in allergy incidence based on “socio-economic status” (as measured by >12 years maternal education) for those of “tropical decent”, (those with maternal birth in Africa, Latin America or Asia) for both those born in Sweden and those born outside of Sweden. Interestingly, there is much less difference based on “socioeconomic status” for those with maternal birth in “temperate” regions (Eastern, Western Europe, and Sweden). Those with mothers from intermediate regions (Middle East, Southern Europe) exhibit higher allergy with “socioeconomic status,” but only for those born in Sweden. The incidence of asthma in those of African decent of “high” “socioeconomic status” born in Sweden is 2.9 times greater than Swedes, roughly the same ratio seen in the US between urban African Americans and suburban (presumably Caucasian) children. Low “socioeconomic status” reduces the incidence to only 1.1 times that of low “socioeconomic” Swedes. Being born outside of Sweden has little protective value for high “socioeconomic status” the incidence still being 2.5 times greater. However, being of low “socioeconomic status” and being born outside of Sweden confers substantial protection, the incidence being only 0.56 that of Swedes. Thus there is a 5 fold difference in incidence of asthma for those of African decent depending on place of birth. It is interesting that the increase in incidence of allergies with increased maternal education parallels the decrease in age of menarche with maternal literacy.

In rural Bavaria Germany, it was found that there was a correlation between the type of fuel used for domestic heating and the development of asthma and other allergies. Heating with coal or wood (compared with central heating) was found to be protective. It was suggested that perhaps cooler bedroom temperatures might explain less sensitization to dust mites, however there was also less sensitization to cats, dogs and pollen. The percentage of homes with cats and with dogs was greater in the coal/wood group. The “socioeconomic status” was lower in the coal/wood group.

Observations such as these have led people to propose the “Hygiene Hypothesis” where increased exposure to allergens or diseases during childhood is believed responsible for protective effects regarding the development of later allergies. However, a consensus statement by a number of professionals at a conference devoted to the Hygiene Hypothesis stated that the data remain conflicting, and there is no indication of which microbe or other agent might be responsible for the protective effects.

Application of AAOB has been found to actually reverse a long standing allergy, namely seasonal hay fever of the inventor. The presence/absence of AAOB may-explain the “contradictory” data in the literature and dermonstrate that it is not contradictory at all. Virtually all studies may be explained through the causal mechanism described here, as is the reason for the sharply increased incidence of allergies for those of tropical decent when born and living in the developed world. It may also explain why low economic status is especially protective when living in regions where bathing practices are a function of economic status. The rural Germans who heated with coal/wood, likely didn't have copious running hot water with which to bathe. It was not how they heated their home that was protective, but instead the shortage of hot water with which to bathe.

The reason that thee agent of the “hygiene hypothesis” has been so elusive is that it does not cause any disease. In fact, the agent cannot cause disease (probably not even in immunocompromised individuals) because it is autotrophic ammonia oxidizing bacteria (AAOB). They do not grow on any heterotrophic media such as is used for isolating pathogens (all of which are heterotrophic as reported by Schechter et al.). (Schechter et al., Mechanisms of Microbial Disease, Williams & Wilkins, Baltimore, Md., USA, 1989.) The only reason they have not been found on the human body is that no one has looked for them with the proper culture media and techniques. They are universally present in all soils where they are responsible for the first step in the oxidation of ammonia into nitrate in the process of nitrification. As autotrophic bacteria, they are incapable of growing anywhere that lacks the substrates they require, ammonia or urea, O2, mineral salts. These substrates are abundantly available on the unwashed skin from sweat residues, and in the “wild” and in the absence of frequent bathing with soap, humans would be unable to prevent the colonization of their external skin with these bacteria. Actually, these bacteria are beneficial, and according to an aspect of the invention, it is appreciated that they are commensal, and that many aspects of human physiology have evolved to facilitate the growth of these bacteria and the utilization of the NO they so abundantly produce.

Another factor that perhaps has prevented their isolation is the bathing practices in developed regions. It has become customary to bath with sufficient frequency so as to prevent the development of body odor. Body odor generally occurs after a few days of not bathing, and the odor compounds are generated by heterotrophic bacteria on the external skin which metabolize exfoliated skin and sweat residues into odiferous compounds. In 3 days, autotrophic bacteria could double approximately 7 times for approximately a 100-fold increase over the post bathing population. In contrast, heterotrophic bacteria could double approximately 200 times for a 10e±60-fold increase. Obviously heterotrophic bacterial growth would be nutrient limited. Assuming similar kinetics of removal through bathing of autotrophic and heterotrophic bacteria, controlling heterotrophic bacteria though bathing would reduce autotrophic bacteria to low, perhaps undetectable levels.

In one embodiment of the invention, it is appreciated that a sufficient population of AAOB on the skin substantially suppresses body odor due to heterotrophic bacteria. The inventor has applied AAOB to his skin and has refrained from bathing for 15 months now, including two summers. There is little body odor associated with sweating. In fact, sweating may decrease body odor by nourishing the AAOB and enhancing their production of NO and nitrite which suppress heterotrophic bacteria. During the winter, with decreased sweating due to low ambient temperatures, there was an increase in odor. However, with increased clothing, (wearing sweaters) the inventor was able to increase basal sweating and reduce body odor to near zero again. There has been no incidents of itching, rashes, skin infections, or athlete's foot infection, and substantially no foot odor.

The AAOB produce nitric oxide as an intermediate in their normal metabolism as reported by Pough et al. (Pough et al., Energy Model and Metabolic Flux Analysis for Autotrophic Nitrifiers, Biotechnol Bioeng 72: 416-433, 2001.) One strain tested by Zart et al. had optimum growth at concentrations of NO in air around 100 ppm (highest level tested in this study). (Zart et al., Significance of gaseous NO for ammonia oxidation by Nitrosomonas eutropha. Antonie van Leeuwenhoek 77: 49-55, 2000.) They can tolerate higher levels. With other strains reported by Schmidt et al., there was no decline in NH3 consumption from 0 to 600 ppm (anaerobic in Ar plus CO2) but it declined by ⅓ at 1000 ppm NO. (Schmidt et al., Anaerobic Ammonia Oxidation in the Presence of Nitrogen Oxides (NOx) by Two Different Lithotrophs, Applied and Environmental Microbiology, November 2002, p. 5351-5357.) Most are aerobic, but some strains can utilize nitrite or nitrate in addition to O2 which increases the NO production. 1000 ppm NO in air corresponds to about 2 μM/L in aqueous solution. The strain used by the inventor has produced a measured NO concentration of 2.2 μM. Most studies of AAOB metabolism have been motivated by their utilization in waste water treatment processes for ammonia and nitrate removal from waste water. Operation of waste water treatment facilities at hundreds of ppm NO is undesirable, so it is not unexpected that the physiology of these bacteria under those conditions has not been well studied.

One mechanism by which AAOB may exert their protective effect on allergies and autoimmune disorders is through the production of nitric oxide, primarily through the regulatory inhibition of NF-κB and the prevention of activation of immune cells and the induction of inflammatory reactions. NF-κB is a transcription factor that up regulates gene expression and many of these genes are associated with inflammation and the immune response including genes which cause the release of cytokines, chemokines, and various adhesion factors. These various immune factors cause the migration of immune cells to the site of their release resulting in the inflammation response. Constitutive NO production has been shown to tonicly inhibit NF-κB by stabilizing IκBα (an inhibitor of NF-κB) by preventing IκBα degradation.

Allergy, asthma, and autoimmune disorders are characterized by an inappropriate, hyper response of the immune system to a particular antigen. This is thought to derive first from an initial “priming” of T-cells either in utero or shortly after birth, followed by priming to a TH2 phenotype, followed by a skewing and polarization of the TH1/TH2 to a TH2 (allergenic) type.

Administration of an NO donor has been shown by Xu et al. to prevent the development of experimental allergic encephalomyelitis in rats. (Xu et al., SIN-1, a Nitric Oxide Donor, Ameliorates Experimental Allergic Encephalomyelitis in Lewis Rats in the Incipient Phase: The Importance of the Time Window. The Journal of Immunology, 2001, 166: 5810-5816.) In this study, it was demonstrated that administering an NO donor, reduced the infiltration of macrophages into the central nervous system, reduced the proliferation of blood mononuclear cells, and increased apoptosis of blood mononuclear cells. All of these results are expected to reduce the extent and severity of the induced autoimmune response.

Allergen exposure is a necessary aspect of sensitization, however there is little evidence that incidence of allergy is directly related to allergen exposure. Exposure to similar quantities of allergens does not always produce similar levels of allergy. Similar levels of asthma occur in populations with very different exposures to the same and different allergens. In a comparison of East and West German levels of allergens prior to unification and subsequent atopic sensitization, the highest exposure levels were in East Germany and the highest levels of atopic sensitization were in West Germany. There is good evidence that allergen reduction prevents allergic response in sensitized individuals, but there is not good evidence causally linking magnitude of allergen exposure to sensitization. For some allergens, there does seem to be a positive dose-response effect (dust mites), but for others, there is an inverse dose-response effect (cat allergies).

According to another aspect of the invention, it is appreciated that inhibition of allergies and autoimmune sensitization may be achieved through topical application of AAOB which produce active NO species in the skin. The exact details of how the immune system works are not fully understood. In general, bacteria, dead or dying cells, foreign organisms, or other debris are first phagocytosed by antigen presenting cells. A major class of these antigen presenting cells are the dendritic cells (DC). These phagocytosed components are digested into smaller fragments, and these fragments are presented to the surface of the antigen presenting cell along with proteins of the major histocompatability complexes I and II (MHC I and MHC II). Immature DC digest the foreign body through either the proteosomal or the endosomal pathway. In the proteosomal pathway, proteins (primarily) from the DC cytoplasm are digested and the resulting antigens are bound to the MHC I. In the endosomal pathway foreign bodies are digested and the resulting antigens bound to the MHC II. The antigens bound to the MHC are then transported to the cell surface where they can interact with T helper cells which come in contact with the antigen presenting cell. In general “self-type” antigens are processed through the proteosomal pathway and “foreign-type” antigens through the endosomal pathway, but there is some cross-priming where and become activated by binding simultaneously to the antigen and the major histocompatability complex. These activated T helper cells, then cause the activation of other immune cells. Gaboury et al. have reported that nitric oxide inhibits mast cell induced inflammation. (Gaboury et al., Nitric Oxide Inhibits Numerous Features of Mast Cell-Induced Inflammation, Circulation. 1996;93:318-326.) Forsythe et al. have shown that nitric oxide inhibits mast cell adhesion through S-nitrosylation of cysteine residues. (Forsythe et al., Inhibition of Captain is a Component of Nitric Oxide-Induced Down-Regulation of Human Mast Cell Adhesion, The Journal of Immunology, 2003, 170: 287-293.) S-nitrosoglutathione (GSNO) strongly down regulated mass cell adhesion. GSNO is the species which would be expected to be formed in the skin from AAOB.

Autism

Low basal NO may lead to autism via the mechanism that new connections in the brain are not “well formed”, and that this malformation of connections is a result of insufficient basal nitric oxide. Insufficient basal nitric oxide may result from a lack of sufficient nitric oxide during the formation and/or refinement of neural connections. Formation and/or refinement of neural connections may predominantly occur during sleep.

Additional symptoms exhibited in autistic individuals may also point to low NO as a cause, including increased pitch discrimination, gut disturbances, immune system dysfunction, reduced cerebral blood flow, increased glucose consumption of the brain, increased plasma lactate, attachment disorders, and humming. Each of these symptoms may be attributed to a low basal NO level.

One method to prevent autism is to increase basal NO levels by restoring the previously unrecognized commensal autotrophic ammonia oxidizing bacteria (AAOB) that in the “wild” (under prehistoric conditions) would live on the scalp and external skin and generate nitric oxide from sweat derived urea. I have previously reported that modern bathing practices wash these bacteria off faster than they can proliferate and the loss of the nitric oxide they generate may cause many of the chronic diseases of the modern world, including hypertension, heart disease, obesity, diabetes, and Alzheimer's Disease. (D. Whitlock, NO production on human skin from sweat derived urea by commensal Autotrophic Ammonia Oxidizing Bacteria, Poster P208, Presented at: The 3rd International Conference on the Biology, Chemistry, and Therapeutic Applications of Nitric Oxide/The 4th annual Scientific meeting of the Nitric Oxide Society of Japan May 24-28, 2004.)

Increasing basal NO levels through the application of AAOB to the external skin may improve some symptoms found in the autism spectrum of disorders. In common with many other people who are successful in science and technology, I consider that I have a mild form of Asperger's Syndrome. Increasing my basal NO level through application of these bacteria has subjectively improved my ability to think creatively, while decreasing my ability to ignore distracting stimuli.

Autotrophic ammonia oxidizing bacteria are universally present in all soils, where they perform the first step in the process of nitrification, the oxidation of ammonia to nitrite. As obligate autotrophs, they are incapable of growth on any standard media used for isolation of pathogens, and may explain why they have not been identified as human commensals earlier, and may not be pathogenic. All known pathogens are heterotrophic. Many animals instinctively cover themselves with dirt and young children also instinctively play in dirt. It may therefore be nearly impossible for humans living in the “wild” in tropical regions where year round sweating occurs to not develop a biofilm containing these bacteria on the external skin. Having such a source of NO continuously available over evolutionary time, humans would evolve to utilize that NO in their physiology. It may be that one physiological reason for non-thermoregulatory sweating is to increase NO production on the skin. All mammals have sweat glands and those mammals that do not thermoregulate via sweating (rats, mice, dogs) have sweat glands concentrated on their feet, perhaps to facilitate prevention of infection by heterotrophic bacteria and fungi. Removal of this NO source through modern bathing practices may cause dysfunction.

Axon Direction, Synaptogenesis in CNS, ANS:

The brain is exquisitely complex and has connections that span many inches. It is well known that neurons are motile, and do move and that axons extend in length, make connections, and retract when misdirected. Inappropriate connections are eliminated and appropriate connections are stabilized. The many connections in the brain are not “random”, but are “programmed” in ways that are not fully understood. Various neurotropic factors are implicated in providing chemical cues for the growth cone of the axon to be repelled from and to “home in on.” No compound has properties that would allow for purely attractive diffusion over a length of several inches. The time constants for diffusion and axon extension cannot be matched to attainable and detectable concentrations.

Therefore, much of the direction of axons may be repulsive, where axons are repelled from inappropriate brain regions. When the growth cone gets “close enough” it can home in using an attractive diffusant. That these connections span several inches, suggests that multiple neurotropic factors are implicated in the long, medium and short range tropism. The number of neurons exceeds the number of possible neurotrophic factors and neurotropic factor receptors Therefore, many of these factors may be used by more than one neuron. The “effective range” of a potential neurotropic factor depends on its production rate, background concentration, destruction rate and diffusion coefficient. The “ideal” attractive compound would be a small molecule with a high diffusivity, a short lifetime, a low background and low detection limit. NO has such properties. Repulsive compounds could be completely immobile and static and some are likely fixed in the cell membrane. The range of an “attractive” compound must be sufficient to reach the target growth cone, but cannot exceed the distance over which a growth cone can accurately register a gradient due to diffusion. A repulsive compound may have zero range and need only work on contact. A growth cone must be repelled at many places along its growth path, but may be attracted to only one site where it forms its terminal connection.

The balance between the extension of a growing axon and the length scale which it can retract when misdirected, may determine a length scale in the developing brain. Presumably, one “characteristic length scale” of the brain is the distance between the last repulsive interaction and the final “correct” connection of a growing axon. Presumably, this length scale is on the same order as the range of the attractive diffusant. An axon need not be connected to a specific cell to function properly. Presumably a connection that is “near enough” may allow for subsequent Hebbian refinement to “improve” the functionality of the connection until it was sufficient.

H-J Song et al. have shown that cyclic nucleotides including cGMP cause a change in a neuronal growth cone from repulsion to attraction. Conversion of neuronal growth cone responses from repulsion to attraction by cyclic nucleotides. Science Vol 281 Sep. 4, 1998. cGMP is produced by guanylyl cyclase when stimulated by NO. Thus NO may provide a signal to signal advancing growth cones to home in. The first few axon connections may be made at “random”, but once some of the appropriate axons have migrated to the proper region, they may stimulate the release of NO in phase with the action potentials in the migrating axons. “Weak” coupling through NO may be transformed to “strong” coupling via synapse formation. Jeseph A. Gally et al. have suggested that NO is the “second messenger which links the activities of neurons in a local volume regardless of whether they are connected by synapses. (Jeseph A. Gally et al., The NO hypothesis: Possible effects of a short-lived, rapidly diffusible signal in the development and function of the nervous system, Proc Natl Acad Sci. USA Vol. 87, 3547-3551, May 1990.)

One of the few neural structures where neural growth and connection making can be observed is in chick embryos. The mapping of connections between the retina and the visual cortex of the chick brain goes through significant refinement during development. Nitric oxide has been shown to be essential for this refinement of the topographic precision of the connectivity. During this refinement, NOS is expressed in target areas of the brain and not in the retina. Hope H. Wu et al. have shown that systemic inhibition of NOS prevents the refinement of connectivity. (Hope H. Wu et al., The role of nitric oxide in development of Topographic precision in the retinotectal projection of chick, J Neurosci. 2001, 21 (12):4318-4325.) Yan He has demonstrated that nitric oxide produces axonal retraction while leaving a thin trailing remnant. (Yan He, Microtubule reconfiguration during axonal retraction induced by nitric oxide, J Neurosci. 2002, 22(14):5982-5991.) This retraction occurred without large scale depolymerization of microtubules and microfilaments. In the presence of brain-derived neurotrophic factor (BDNF) NO stabilizes neuronal growth cones. Alan F. Ernst et al. stabilized growth cones in contact with BDNF coated beads against NO-induced retraction. (Alan F. Ernst et al., Stabilization of growing retinal axons by the combined signaling of nitric oxide and brain-derived neurotrophic factor, J Neurosci 2000, 20(4):1458-1469.) Other factors, nerve growth factor (NGF) and neurotrophin-3 (NT-3) did not prevent NO induced growth cone collapse. Hope H. Wu et al. showed that inhibition of NOS increases the number of ipsilaterally projecting ganglion cells by 1000% over controls, yet only 10% of them survived. (Hope H. Wu et al., Involvement of nitric oxide in the elimination of a transient retinotectal projection in development, Science; Sep. 9, 1994; 265, 5178.) P. Cammpello-Costa et al. showed that blockage of NOS induces increased errors in connectivity and increases lesion-induced plasticity in the rat retinotectal projection. (P. Cammpello-Costa et al., Acute blockade of nitric oxide synthesis induces disorganization and amplifies lesion-induced plasticity in the rat retinotectal projection, J. Neurobiol 44:371-381, 2000.)

Marriann Sondell et al. have shown that axon growth is stimulated by VEGF. (Marriann Sondell et al., Vascular Endothelial Growth Factor Has Neurotrophic Activity and Stimulates Axonal Outgrowth, Enhancing Cell Survival and Schwann Cell Proliferation in the Peripheral Nervous System, The Journal of Neuroscience, Jul. 15, 1999, 19(14):5731-5740.) VEGF transcription is initiated by HIF-1α, which is initiated by the combined signal of low O₂ and high NO as illustrated by Greg L. Semenza in HIF-1α: mediator of physiological and pathophysiological responses to hypoxia, Invited Review (J. Appl Physiol 88: 1474-1480, 2000); and by Sandau et al. in Accumulation of HIF-1α under the influence of nitric oxide, (Blood. 2001;97:1009-1015.) Blood flow is known to be strongly correlated with neural activity. Vasodilatation may be mediated through NO activation of guanylyl cyclase and cGMP production leading to relaxation of vascular smooth muscle. Neuronally generated NO may provide the signal to initiate transcription of VEGF and stimulate angiogenesis as well as to couple blood supply with neural activity. With the “sink” for NO being oxygenated hemoglobin, there may be a natural feedback mechanism to prevent “too much” angiogenesis. The factor that controls brain angiogenesis may be limited to molecules that the blood brain barrier is permeable to, such as NO. Kon et al. have shown that inhibition of NOS retards vascular sprouting in angiogenesis. Nitric oxide synthase inhibition by N(G)-nitro-L-arginine methyl ester retards vascular sprouting in angiogenesis. (Kon et al., Microvascular research 65 (2003) 2-8.) Toshiro Matsunaga et al. have shown that ischemia induced growth of cardiac collateral vessels requires eNOS and NO. Ischemia-induced coronary collateral growth is dependent on vascular endothelial growth factor and nitric oxide. (Circulation 2000;102:3098-3103.) Dong Ya Zhu has shown that neurogenesis following focal cerebral ischemia requires nitric oxide, and is absent in adult mice lacking the iNOS gene. (Dong Ya Zhu et al., Expression of inducible nitric oxide synthase after focal cerebral ischemia stimulates Neurogenesis in the adult rodent dentate gyrus, J. Neurosci. Jan. 1, 2003 23(1):223-229.) Presumably, neurogenesis at other times may also require NO. J. D. Robertson et al., have reported that inhibition of nitric oxide synthase blocks tactile and visual learning in the octopus. (J. David Robertson, et al. Nitric oxide is required for tactile learning in Octopus vulgaris, Proc. R. Soc. Lond. B (1994) 256, 269-273; and J. David Robertson et al., Nitric oxide is necessary for visual learning in Octopus vulgaris, Proceedings; Biological Sciences, Vol. 263, No. 1377 (Dec. 22, 1996), 1739-1743.)

Many neural connections in the brain are “well formed.” Presumably, to achieve this, there may be a mechanism whereby connections can be “tested” and “correct” connections stabilized and “incorrect” connections removed. Presumably, the development of a particular neural structure may involve the proliferation of the relevant cells, projection of axons to the relevant brain volumes, repulsion from inappropriate volumes, connection to the appropriate cells, feedback inhibition of proliferation, followed by pruning of excess or misconnected cells. Presumably the length scale at which these connections can occur depends on the range of the diffusive attractant the migrating axons use to home in on. If that diffusive attractant is NO, anything that lowers the range of NO diffusion may decrease the volume size of brain elements that can be “well connected.” A brain which developed under conditions of low basal NO levels may be arranged in smaller volume elements because the reduced effective range of NO.

NO has been implicated as a volume signaling molecule. A unique feature of NO, as a very small hydrophobic molecule is that it can diffuse large distances compared to other neurotransmitters and pass through lipid membranes and through the blood-brain barrier. The distance which NO can diffuse and achieve a certain terminal concentration depends on the background concentration of NO. The diffusing signal of NO may add to the background NO concentration, and when the sum exceeds the action level, the action of the NO signal may occur. When a signal produces a specific quantity of NO, the range of that signal may depend on the NO background. With a lower background, the quantity of NO required to raise a volume to the action level may be increased. Alternatively, the volume which an NO signal can affect may be reduced when the NO background is lower, or in other words, the effective range of the NO signal may be reduced.

The background concentration dependence on the range of action of NO may explain some effects seen in autism. Some autistic individuals exhibit superior auditory pitch discrimination, reduced auditory “global interference,” and/or increased discrimination of “false memories.” So called “savant” type abilities are not uncommon. A change in the “homing range” distance for protecting axons may produce improved neural processing of “simple” tasks by increasing local short distance neural connection density in areas providing that “simple” mental function, but it may occur at the expense of more “complex” tasks which require integration of multiple processes over larger volumes through connections spanning longer distance.

Dr. E. H. Aylward et al., has reported that autistic individuals, in their limbic system, have decreased neuron size, increased neuron density, and reduced dendrite complexity. (E. H. Aylward, PhD et al., MRI volumes of amygdala and hippocampus in non-mentally retarded autistic adolescents and adults, Neurology 1999;53:2145.)

Similarly, M. F. Casanova et al, have reported that cells in minicolumns are reduced in size but increased in number. (Manuel F. Casanova, et al., Minicolumnar pathology in autism, Neurology 2002;58:428-432.) It is also reported by D. G. Amaral et al, that in the amygdala, cells are reduced in size, but increased in number density. (D. G. Amaral, M. D. et al., The amygdala and autism: implications from non-human primate studies, Genes, Brain and Behavior (2003) 2: 295-302 Review.) In fMRI comparisons of autistic and dyslexic brains, similarities have been noted in white matter volume excesses. M. R. Herbert et al. have shown that global volume excesses are observed in autistic individuals, and volume excesses in the parietal lobes are observed in dyslexics. (Martha R. Herbert et al., Localization Of White Matter Volume Increase In Autism And Developmental Language Disorder, Ann. Neurol 2004; 55:530-540.) While some autistic individuals are also dyslexic, rarely autistic individuals are hyperlexic. In one case reported by Peter E. Turkeltaub et al., an autistic boy learned to read before he could speak, and his first spoken word was a word he read. (Peter E. Turkeltaub, et. al., The neural basis of hyperlexia reading: an fMRI case study, Neuron, vol 41, 11-25, Jan. 8, 2004.) Autistic individuals showing greater skill in tests such as Block Design have led people, such as H. Tager-Flusbert et al., to propose the weak central coherence hypothesis, that there is inadequate connectivity between different components of the brain, and this inadequate connectivity translates into impaired ability to process gestalts. (Helen Tager-Flusberg, et al, Current Directions in Research on Autism, Mental Retardation and Development disabilities Research Reviews 7: 21-29 (2001).)

NO may work in concert with NMDA receptors. Excessive NO production inhibits NMDA receptors, which is reported by A. Contestabile to be involved in the feedback control of neuron excitability. (Antonio Contestabile, Role of NMDA receptor activity and nitric oxide production in brain development, Brain Research Reviews 32(2000) 476-509.) M. Virgili et al report that neonatal blockage of NMDA receptor in rats results in long term down regulation of NNOS. (M. Virgili et al., Neuronal nitric oxide synthase is permanently decreased in the cerebellum of rats subjected to chronic neonatal blockade of N-methyl-D-aspartate receptors, Neurosci Lett. 258 (1988) 1-4.) R. J. Nelson et al demonstrated that nNOS knock-out mice and mice treated with NNOS inhibitors display excessive aggression toward other mice. R. (J. Nelson et al. Behavioral abnormalities in male mice lacking neuronal nitric oxide synthase, Nature 378 (1995) 383-386.) NO may therefore be important in neuronal proliferation, neuronal migration, synaptogenesis. Presumably disruption in NO metabolism may have multiple effects in neural development.

Nitric oxide has been demonstrated by Klyachko et al, to increase the excitability of neurons by increasing the after hyperpolarization through cGMP modification of ion channels. (Klyachko et al., cGMP-mediated facilitation in nerve terminals by enhancement of the spike after hyperpolarization, Neuron, Vol. 31, 1015-1025, Sep. 27, 2001.) C. Sandie et al. have shown that inhibition of NOS reduces startle. (Carmen Sandi et al., Decreased spontaneous motor activity and startle response in nitric oxide synthase inhibitor-treated rats, European journal of pharmacology 277 (1995) 89-97.) Attention-Deficit Hyperactivity Disorder (ADHD) has been modeled using the spontaneously hypertensive rat (SHR) and the Naples high-excitability (NHE) rat. Both of these models have been shown by Raffaele Aspide et al, to show increased attention deficits during periods of acute NOS inhibition. (Raffaele Aspide et al., Non-selective attention and nitric oxide in putative animal models of attention-deficit hyperactivity disorder, Behavioral Brain Research 95 (1998) 123-133.)

Inhibition of NOS has also been shown by M. R. Dzoljic to inhibit sleep. (M. R. Dzoljic et al., Sleep and nitric oxide: effects of 7-nitro indazole, inhibitor of brain nitric oxide synthase, Brain Research 718 (1996) 145-150.) G. Zoccoli has reported that a number of the physiological effects seen during sleep are altered when NOS is inhibited, including rapid eye movement and sleep-wake differences in cerebral circulation. (G. Zoccoli, et al., Nitric oxide inhibition abolishes sleep-wake differences in cerebral circulation, Am. J. Physiol. Heart Circ Physiol 280: H2598-2606, 2001.) NO donors have been shown by L. Kapas et al. to promote non-REM sleep, however, these increases persisted much longer than the persistence of the NO donor, suggesting perhaps a rebound effect. (Levente Kapas et al., Nitric oxide donors SIN-1 and SNAP promote nonrapid-eye-movement sleep in rats, Brain Research Bullitin, vol 41, No 5, pp.293-298, 1996.) M. Rosaria et al., Central NO facilitates both penile erection and yawning. (Maria Rosaria Melis and Antonio Argiolas, Role of central nitric oxide in the control of penile erection and yawning, Prog Neuro-Psychopharmacol & Biol. Phychiat. 1997, vol 21, pp 899-922.) P. Tani et al, have reported that insomnia is a frequent finding in adults with Asperger's. (Pekka Tani et al., Insomnia is a frequent finding in adults with Asperger's syndrome, BMC Psychiatry 2003, 3:12.) Y. Hoshino has also observed sleep disturbances in autistic children. (Hoshino Y et al., An investigation on sleep disturbance of autistic children. Folia Psychiatr Neurol Jpn. 1984;38(1):45-51(abstract).) K. A. Schreck et al. has observed that the severity of sleep disturbances correlates with severity of autistic symptoms. (Schreck K A, et al., Sleep problems as possible predictors of intensified symptoms of autism, Res Dev Disabil. January-February 2004;25(1):57-66 (abstract).)

It may be that high NO levels are essential for sleep, and that these high NO levels are also necessary for the neural refinement that may occur during sleep. Night time may be an ideal time to administer large doses of NO to the brain. Basal metabolism is at its lowest level, therefore, there may be maximum metabolic reserves to compensate for NO induced hypotension and NO induced inhibition of cytochrome oxidase. The individual subject is immobile so the brain need not function to control physical activity. The individual subject is unconscious so the brain need not function to integrate sensory data. It may be that during this night time surge in NO that much of long term potentiation occurs. A large surge in NO may serve to cause misdirected axons to retract, and may strengthen newly formed synapses. The brain activity that occurs during sleep could serve to exercise the newly formed synapses so as to impedance match and optimize the various connections. Using a global mechanism from outside the brain, such as night time sweating on the scalp, may relieve the brain of local regulation of basal nitric oxide level.

It may further be that high levels of NO during sleep may be part of the “normal” “housekeeping” functions of the brain, and may serve in general to refine connections, make short term memory permanent, and “optimize” brain function. It may be that the neural activity that accompanies REM sleep is part of the 'testing” of neural connections necessary to “decide” which ones to keep and which ones to ablate. High levels of NO during sleep may be necessary for sleep to be effective for these “housekeeping” functions. It is these high levels of NO generated in part by neural activity of the sleeping brain that may be responsible for the drop in blood pressure observed during sleep. Adrenergic sweating at night, particularly on the scalp, causes the release of urea to the scalp where autotrophic ammonia oxidizing bacteria (AAOB) would generate NO.

S. Ogawa has reported that blood flow in the brain is closely coupled with neural activity, and this close coupling is the basis for fMRI studies where prompt (sub second) alterations in hemoglobin oxygenation (increase in O₂ level) can be correlated with neural activity. (Seiji Ogawa, et al., An approach to probe some neural systems interaction by functional MRI at neural time scale down to milliseconds. PNAS Sep. 26, 2000. vol 97 no 19, 10661-10665.) In the peripheral circulation, blood flow may be regulated though NO mediated activation of guanylyl cyclase and cGMP mediated relaxation of vascular smooth muscle. Presumably a similar mechanism may hold for the brain vasculature as well. NO generated from neuronal activity may provide NO to relax vascular smooth muscle. However, the promptness of changes in hemoglobin oxygenation might suggest changes in O₂ consumption (by inhibition of cytochrome oxidase by NO) rather than increased supply (though vasodilatation mediated flow increase). Since mitochondria are regulated by NO, and the operating point of mitochondria is fixed by the instantaneous concentrations of both O2 and NO, any increase in NO may decrease mitochondria activity. Both effects of NO may likely occur simultaneously.

It may also be that measuring NO levels, namely the ratio of NO/O₂ may provide a better measure of the “O₂ diffusive closeness” to O₂Hb, and hence the regulation of capillary spacing in the brain. Presumably, the “O₂ diffusive closeness” of a particular site to oxygenated hemoglobin (O₂Hb) (the source of O₂) must be measured and angiogenesis initiated when it is too low, and capillaries ablated when it is too high. However, it may be that simply measuring the O₂ level is inadequate because the detection of pathologically inadequate perfusion would necessitate pathological O₂ levels. Also, areas with adequate capillary density may not be distinguished from areas with excess capillary density because in both cases O₂ levels are adequate. Measuring NO levels would provide a better measurement. NO has a diffusivity very similar to that of O₂. O₂Hb is the source of O₂, and is also the sink for NO, where O₂Hb destroys NO with diffusion limited kinetics. Low NO may therefore be the “signal” that indicates adequate “O₂ diffusive closeness.” Low basal NO may lead to the capillary rarefaction observed in many disorders, including hypertension and diabetes. Low basal NO in the brain may lead to capillary rarefaction and hypoperfusion, as well as the characteristic white matter hyperintensity observed in fMRI and which accompanies many neurological disorders. High local levels of NO due to neural activity may signal both the greater innervation of those areas by nearby growing axons, and also greater vascularization through angiogenesis.

Takashi Ohnishi et al. have reported that autistic individuals show decreased blood flow. (Takashi Ohnishi et al., Abnormal regional cerebral blood flow in childhood autism, Brain (2000), 123, 1838-1844.) J. M. Rumsey et al. have reported that autistic individuals have increased glucose consumption. (Rumsey et al., Brain metabolism in autism, Resting cerebral glucose utilization rates as measured with positron emission tomography. Arch Gen Psychiatry, May 1985;42(5):448-55 (abstract).) D. C. Chugani has reported that autistic individuals have an increased plasma lactate levels. (Chugani D C, et al., Evidence of altered energy metabolism in autistic children, Prog Neuropsychopharmacol Biol Psychiatry. May 1999;23(4):635-41.) The occurrence of these effects may be a result of capillary rarefaction in the brain, which may reduce blood flow and O₂ supply, such that some of the metabolic load of the brain may be produced through glycolysis instead of oxidative phosphorylation. Glycolysis consumes 19 times more glucose than oxidative phosphorylation does to produce the same ATP and produces lactate. While neurons don't produce ATP through glycolysis, other cells in the brain do, namely astrocytes. Capillary rarefaction may both decrease blood flow and increase glucose consumption and increase lactate generation.

It may be that a lack of NO during certain critical periods of development interferes with the formation of high fidelity and efficient neural connectivity over certain length scales. The impairment in connectivity observed in chick visual cortex when basal NO is lowered through NOS inhibition, may also occur in humans when basal NO is reduced by whatever means. Presumably, other neurons use the same NO mediated mechanism that is utilized in the visual cortex. High levels of local connectivity may provide for superior processing of simple neural tasks, at the expense of an inability to integrate those simple tasks into a whole.

Percolation and Critical Connectivity

Much of the brain is essentially a two dimensional association of individual minicolumns. The main difference between human and animal brains is not the structure of the individual minicolumns, but the greatly increased number and connectivity in humans. Presumably, it is the connectivity of those individual minicolumns that produces the “emergent” human characteristics, such as language, that distinguish humans from animals. If the association of minicolumns is looked at as a connected network, the connectivity of that network may be represented by a length scale. G. Grimmett reported that near the percolation threshold, the overall connectivity of a network becomes very sensitive to small changes in local connectivity. (Geoffrey Grimmett, Percolation, Springer-Verlag, 1989.) Every element in a functioning neural network cannot be connected to every other element. Neither can every element be disconnected. As the degree of connectivity changes, the degree of connectivity where the properties of the network change most rapidly is at the percolation threshold, where “critical” behavior is observed. That is, various properties of the network diverge at the percolation threshold. For example, slightly below the percolation threshold the length scale of the largest connected cluster is finite; slightly above the threshold is it infinite. Presumably, the neural network that forms the brain may be above the percolation threshold. Otherwise there would be regions of the brain that are not connected. The brain is not a “simple” network. There are multiple neurotransmitters, perhaps each representing a different network.

It may be that NO, acts as a coupling agent between the various (somewhat) independent networks. “Weak” coupling with NO may facilitate axonal migration and neurogenesis and the formation of “strong” coupling through formation of synapses at the exact “right spot.” Some parts of the brain may likely be close to the percolation threshold. There is no strong advantage to a degree of connectivity much higher than the percolation threshold. Connectivity much higher than the percolation threshold is likely to increase the stability of the network, but at the expense of sensitivity of that network to change. Autistic individuals may simply have a slightly too low a degree of local connectivity, which may be brought about by a low basal NO-level. Below the percolation threshold, the functionality of a network may be expected to degrade rapidly.

Decreased stability of a neural network would cause increased vulnerability to seizures and it is noted that autistic individuals do have a greater incidence of seizures. Interestingly, I. T. Demchenko et al. have reported that hyperbaric O2 reduces cerebral NO levels and also induces seizures. (Ivan T. Demchenko, et. al., Hyperbaric O2 reduces cerebral blood flow by inactivating nitric oxide. Nitric oxide: Biology and Chemistry vol 4, No. 6, 597-608 (2000).) NOS inhibitors increase the latency to seizure as does L-arginine however, the NO donor S-nitroso-N-acetylpenicilamine (SNAP) significantly shortens it as reported by N. Bitterman. (Noemi Bitterman et al., L-Arginine-NO pathway and CNS O₂ toxicity, J Appl Physiol 84 (5): 1633-1638, 1998.) NOS does generate NO, however it can also generate superoxide which destroys NO. NOS inhibitors may block both NO and superoxide production. When NO and superoxide are produced together, peroxynitrite is produced. Peroxynitrite may oxidize the Zn-thiolate group in the NOS complex and “uncouple” NOS leading to superoxide formation. Thus the effect of NOS inhibitors on seizure thresholds may be due to its blocking of superoxide formation and not due to blocking of NO formation.

One can look at the brain as a number of somewhat independent processes such as visual processing, auditory processing, individual primitive function generation, language, motor, ANS, etc. Presumably each of these different “functions” may require an individual brain structure. Presumably that individual brain structure may be a local network with some degree of local connectivity. The percolation threshold for a network may be a critical point. Near the percolation threshold, the properties of the network change exponentially, that is it requires an exponentially smaller and smaller change to effect a macroscopic change in the network the closer to the percolation threshold one is. Presumably different brain structures may require different degrees of connectivity to accomplish the required function. Presumably, for relatively “simple” functions like sensory processing “robust” operation is more important than extreme sensitivity to change. Such structures likely have connectivity well above the critical percolation level. Greater computational effectiveness, such as for functions such as creativity, may require connectivity closer to the percolation threshold. It has been suggested that a “touch” of autism or Asperger's can contribute to intelligence and to creativity. (Ed. Uta Frith, Elisabeth Hill. Autism: Mind and Brain, Oxford University Press: 2003, reviewed Nature 428, Apr. 1, 2004, 470-471.) A quote attributed to Hans Asperger, “it seems that for success in science or art a dash of autism is essential.” (Allan Snyder, Autistic genius? Book review: Nature 428, Apr. 1, 2004, 470-471.) Perhaps the increased abilities of autistic individuals in some mental areas may be derived from a reduced connectivity in those brain structures leading to a closer approach to the percolation threshold and greater sensitivity to change. A reduced connectivity length is only helpful to a point. Once the percolation threshold is reached, the functionality of the network may rapidly degrade.

If reduced connectivity is the problem in autistic brains, increasing the connectivity may be expected to improve function. If the connectivity is in the near percolation threshold region, the change may be exponential, highly non-linear and improvement may be dramatic.

Impaired ability to “see” gestalts may extend into other areas as. well. The inability to perceive “shades of grey”, to perceive things as either “black or white”, may derive from a lessened ability to integrate numbers of diverse stimuli (or primitive elements) into a whole. Obsessive attachment to specific objects may derive from a similar collapse of the responding brain structures to highly local tiny areas. A significant component of the volume of the brain consists of axons which join different brain regions. Efficient connectivity may minimize path length and minimize axon volume. Inefficient connectivity may result in increased brain volume without an increase in functionality. The increased brain size observed in autistic children may be a measure of inefficient connectivity.

N. Schweighofer et al. have reported that diffusion of NO can facilitate cerebellar learning. (Nicolas Schweighofer et al., Diffusion of nitric oxide can facilitate cerebellar learning: A simulation study. PNAS Sep. 12, 2000, vol 97, no. 19, 10661-10665.) This was a simulation study that showed that plausible NO concentrations and diffusion properties could improve error correcting. M. F. Casanova et al. have reported that there is an increased density of smaller minicolumns in autism. (Manuel F. Casanova et al., Minicolumnar pathology in autism. Neurology 2002; 58:428432.) Low NO background may decrease the range at which a NO signal may act, and perhaps provides a rational for the increased density of smaller minicolumns. Just as there may be a signal to initiate neurogenesis, there may also be a signal to stop neural proliferation. NO could provide both signals. A high level of NO close to a source may initiate proliferation, and a low level of NO at the distance where diffusion lowers the NO concentration may terminate it. Tenneti et al. have reported that S-nitrosylation of neural caspase has been shown to inhibit neuronal apoptosis. (Lalitha Tenneti et al., Suppression of neuronal apoptosis by S-nitrosylation of caspases. Neuroscience Letters 236 (1997) 139-142.) E. Ciani et al., have reported that NO protects neuroblastoma cells from apoptosis due to serum deprivation. (Elisabetta Ciani et al., Nitric oxide protects neuroblastoma cells from apoptosis induced by serum deprivation through cAMP-response element-binding progein (CREB) activation, J Bio Chem, 277 (51) 49896-49902, 2002.) C. Nucci et al. have reported that NO may be implicated in diverse roles in the lateral geniculate nucleus, from signal transduction to both causing and preventing neuronal apoptosis. (C. Nucci et al., Multifaceted roles of nitric oxide in the lateral geniculate nucleus: from visual signal transduction to neuronal apoptosis, Toxicology letters 139 (2003) 163-173.)

The brain is not the only place where neuronal connections are made during early childhood. One of the reasons that infants are incontinent is that they lack neuronal control of the voiding functions. Just as the voluntary muscles must be properly innervated to function, so too the various smooth muscles and visceral organs must be connected to the autonomic nervous system (ANS) to function properly. Part of the inability of infants to digest adult foods may derive from a lack of control of the various digestive organs by the ANS. Some of the digestive disturbances seen with autism may derive from a lack of the proper connectivity of the ANS to the viscera. D. Blottner has implicated Nitric oxide as a messenger in the ANS where nitrinergic pathways are important. (Dieter Blottner, Nitric oxide and target-organ control in the autonomic nervous system: Anatomical distribution, spatiotemporal signaling, and neuroeffector maintenance, J Neurosci Res. 58:139-151 (1999).) H. Matsuama et al. have reported that vasoactive intestinal protein (VIP) release is regulated by NO. (H. Matsuyama Et Al., Peptidergic and Nitrergic Inhibitory Neurotransmissions In The Hamster Jejunum: Regulation Of Vasoactive Intestinal Peptide Release By Nitric Oxide, Neuroscience Vol. 110, No. 4, pp. 779-788, 2002.)

D. Blottner has also reported that Nitric oxide is involved in trophic mechanisms in the maintenance and plasticity of the autonomic nervous system. (Dieter Blottner, Nitric Oxide and Target-Organ Control in the Autonomic Nervous System: Anatomical Distribution, Spatiotemporal Signaling, and Neuroeffector Maintenance, Journal of Neuroscience Research 58:139-151 (1999).) E. Niebergall-Roth et al. reported that release of digestive enzymes by the pancreas is controlled in part by the ANS. (E. Niebergall-Roth et al., Central and peripheral neural control of pancreatic exocrine secretion, Journal of physiology and pharmacology 2001, 52, 4, 523-538.) H. E. Raybould also reported that release of digestive enzymes is also regulated by compositional feedback from sensors in the gut. (Helen E. Raybould. Does your gut taste? Sensory transduction in the gastrointestinal tract, News Physiol. Sci. vol 13, December 1998, 275-280.)

Presumably, improper innervation of the gut by the ANS may impair function. T. Wester et al. have shown that the density of neurons in the gut staining positive for NADPH diaphorase (equivalent to NOS) drops markedly in early childhood, and that “nitric oxide is the most important transmitter in non-adrenergic non-cholinergic nerves in the human gastrointestinal tract.” (T. Wester et al., Notable post natal alterations in the myenteric plexus of normal human bowel, Gut 1999;44:666-674.)

Nitric Oxide Involvement in Attachment:

NO is involved in the development of the bonding and smell recognition that occurs in ewes within 2 hour of giving birth. K. M. Kendrick et al., showed that inhibition of rNOS blocks formation of olfactory memory, and this blockage can be reversed by infusion of NO into the olfactory bulb. (Kendrick K M et al., Formation of olfactory memories mediated by nitric oxide, Nature, Aug. 14, 1997;388(6643):670-4.) J. N. Ferguson et al. reported that oxytocin is essential in the formation of normal social attachment in mice. (Jennifer N. Ferguson et al., Oxytocin in the medial amygdale is essential for social recognition in the mouse, Journal Neuroscience, Oct. 15, 2001, 21 (20):8278-8285.) G. L. Williams et al. reported that a reduction in oxytocin release following epidural anesthesia in heifers preceded a reduction in maternal bonding type behaviors. (G. L. Williams et al., Physiological regulation of maternal behavior in heifers: Roles of genital stimulation, intracerebral oxytocin release and ovarian steroids, Biology of Reproduction 65, 295-300 (2001).) G. Gimpl et al. reported that activation of the oxytocin receptor causes activation of nitric oxide synthase. (Gerald Gimpl et al., The oxytocin receptor system: structure, function, and regulation, Physiological reviews vol. 81, No. 2, 629-683, April 2001.) S. K. Mani et al. reported that inhibition of nitric oxide synthase inhibits lordosis in progesterone stimulated estrogen primed ovariectomized rats. (Shailaja K. Mani, et al., Nitric oxide mediates sexual behavior in female rats, Proc Natl Acad Sci, Vol. 91, 6468-6472, July 1994.)

W. D. Ratnasooriya et al reported that inhibition of NOS in male rats reduces pre-coital activity, reduces libido, and reduces fertility. (W. D. Ratnasooriya et al., Reduction in libido and fertility of male rats by administration of the nitric oxide (NO) synthase inhibitor N-nitro-L-arginine methyl ester, International journal of andrology, 23: 187-191 (2000).) R. R. Ventura et al. reported that nitric oxide modulates the activity of oxytocin and vasopressin in the regulation of sodium and water balance. (R. R. Ventura, et al., Nitrergic modulation of vasopressin, oxytocin, and atrial natriuretic peptide secretion in response to sodium intake and hypertonic blood volume expansion, Brazilian journal of medical and biological research (2002) 35: 1101-1109.) Thus nitric oxide may be involved in pathways known to be important in attachment.

The neurological changes that occur during attachment, either maternal bonding or pair bonding following intercourse can be robust and long lasting, indicating “well formed” connections. C. O. Okere et al. reported that these connections can occur in the space of a few hours. (Okere and Kaba, Increased expression of neuronal nitric oxide synthase mRNA in the accessory olfactory bulb during the formation of olfactory recognition memory in mice, Eur J Neurosci. December 2000;12(12):4552-6.) The distance over which axons must migrate to form these new connections may therefore be limited. If the “attachment” neural connections are formed during a period of low NO, perhaps those connections may only be formed in a very local area, thereby forming a powerful “attachment”, but perhaps one that may not be modulated by input from other areas. Perhaps this may also lead to dysfunctional attachments, attachment to abusers, attachments to inanimate objects, and perhaps obsessive compulsive behavior.

“Attachment” is in some senses “programmed”. Humans (and other animals) are “programmed” to attach to their offspring and to their mates. This characteristic response can occur rapidly (hours in ewes), shorter than the time for neurogenesis, indicating that the behavior originates from neurons that are already present, but that they become connected in different ways during that time.

Immune System Interactions

The onset of autistic symptoms in children has been anecdotally associated with childhood vaccinations. While epidemiologic studies have shown no change in incidence in large populations coincident with MMR use or disuse. A consequence of vaccination and activation of the immune system is release of cytokines and induction of iNOS. Elevated plasma nitrate is associated with stimulation of the immune system and is a consequence of iNOS induction. INOS transcription is mediated through NFκB. M. Colasanti et al. have reported that NFκB is inhibited by NO and so INOS transcription is inhibited by NO. (Marco Colasanti et al., Induction of nitric oxide synthase mRNA expression suppression by exogenous nitric oxide, J Bio Chem 270, 45, 26731-26733, 1995.) Thus a low basal NO level may cause increased INOS expression and increased NO levels during immune activation (over levels reached with a higher basal NO level). Because INOS is regulated with a “feed forward” type regulation, if too much INOS is generated, NO levels may rise to pathological levels, as in septic shock.

iNOS induction may have an effect on neuronal signaling. Increased background of NO may lower the amount on NO necessary to produce effects and may increase the range at which these effects could occur. Effects of NO mediated through nNOS and eNOS would occur at lower thresholds of NO production. Feedback inhibition of nNOS and eNOS transcription may likely occur at lower nNOS and eNOS expression. U. Forstermann et al. have reported that in vitro following treatment with bacterial lipopolysaccharide (which causes expression of INOS), that nNOS expression is down regulated. (Ulrich Förstermann et al., Expressional control of the ‘constitutive’ isoforms of nitric oxide synthase (NOS I and NOS III), FASEB J. 12, 773-790 (1998).) After the INOS induced increase in basal NO, basal NO may fall to pre-iNOS levels (or lower). nNOS is synthesized in the cell body, in the endoplasmic reticulum, and is then transported to the site of activity through the axon. This transport necessarily takes some time. Reduced nNOS transcription by high NO levels following immune stimulation during low NO levels may cause NO levels to drop still further. S. H. Fatemi have demonstrated that prenatal viral infection of mice has been demonstrated to produce long term increases and-decreases in nNOS expression in different mouse brain regions. (Fatemi SH et al., Prenatal viral infection causes alterations in NNOS expression in developing mouse brains, Neuroreport. May 15, 2000;11(7):1493-6 (abstract).)

For NO to function as a transmitter between cells, it is necessary that NO be produced at one cell and be detected at another cell. Production of NO by a cell is regulated within that cell and is also regulated by receptors on the surface of the cell. There are very few molecules that diff-use as fast as NO. Feedback regulation of NO production by a cell with a non-NO transmitter, may necessarily entail a significant time lag during which time the NO production would be unregulated and could reach supraphysiological levels.

However, immunizations are not the only sources of immune system activation leading to iNOS induction during early childhood. Early childhood is characterized by many infections, colds, runny noses, diarrheas. While perturbation of NO metabolism might occur as a consequence of any particular immunization, it might equally occur as a consequence of any other immune stimulation. Thus MMR vaccination could be the proximate “cause,” for a susceptible individual, but in the absence of MMR, some other immune stimulation, perhaps one of the many diseases of childhood, may invariably initiate the change in NO metabolism. Thus the absence of changes in incidence of autism observed in large populations may result from a myriad of other immune system stimulation events of early childhood being equally effective at triggering the autism response in susceptible individuals.

If there is a causal chain between vaccination and autism, a NO mediated pathway may be a conceivable link in that causal chain. However, is it unclear whiter it is the high levels reached during immune stimulation, and/or the low level post vaccination that initiates autistic symptoms. Low levels post iNOS stimulation likely initiate autistic symptoms. Development does not occur all at once, but it is an ongoing process. Any disturbance to that process may likely be ongoing as well. In the absence of AAOB generated NO, basal NO levels may become unstable. Low NO leads to increased iNOS expression during immune stimulation and a drop in eNOS and nNOS leading to still lower basal NO levels. Thus, each instance of immune stimulation could cause the basal NO level to ratchet lower. In the “wild” chronic infection with parasites or colonization of the skin with AAOB may exert a stabilizing effect on basal NO levels. The desire of individuals in developed regions to remain free from parasites may increase susceptibility to other disorders. Similarly, a biofilm of AAOB may raise basal NO levels and exert a stabilizing effect on NO levels.

Dr. N. A. Halsey et al. reported that an immune system deviation has been observed in autistic children, characterized by a decrease in Th1 cells and an increase in Th2 cells. (Neal A. Halsey et al., Measles-Mumps-Rubella Vaccine and Autistic Spectrum Disorder: Report From the New Challenges in Childhood Immunizations Conference Convened in Oak Brook, Ill., Jun. 12-13, 2000, Pediatrics 2001; 107(5).URL:http://www.pediatrics.org/cgi/content/full/107/5/e84.) R. C. van der Veen et al noted that Th1 cells, when incubated with antigen, generate NO which inhibits T cell proliferation. (Roel C. van der Veen, et al., Antigen Presentation to Th1 but Not Th2 Cells by Macrophages Results in Nitric Oxide Production and Inhibition of T Cell Proliferation: Interferon-γ is essential but insufficient, Cellular Immunology 206, 125-135 (2000) doi:10.1006/cimm.2000.1741, available online at http://www.idealibrary.com.) C. S. Benn et al reported that immune system deviation has been seen to increase with increased number of serious infections in early childhood. (Christine Stabell Benn et al., Cohort study of sibling effect, infectious diseases, and risk of atopic dermatitis during first 18 months of life, BMJ, doi:10.1136/bmj.38069.512245.FE (published 30 Apr. 2004).) Thus a “NO ratchet” in children may lead to a progressively worse immune deviation.

Cellular ATP and Energy Depletion may be a Consequence of Nitropenia

ATP is the cell's major energy transfer species. When ATP is cleaved to ADP+Pi, energy is released, and many physiological processes couple that energy to the performance of energy consuming processes. Virtually all of the cell's metabolic processes require ATP, and if ATP levels fall too low, a cell will invariably deteriorate and ultimately die. ATP production and regulation is thus critically important, and there are multiple redundant mechanisms for ATP production and regulation. However, a number of these are regulated via NO mediated processes, and when there is insufficient NO, or nitropenia, one consequence is a lowered basal ATP-level. As used herein the term “nitropenia” is used to describe low basal nitric oxide.

Since virtually all metabolic processes utilize ATP, insufficient ATP will compromise virtually all cellular functions. A reduction in ATP can lead to apoptosis, and if severe, to necrosis. Such apoptosis and necrosis would be expected at those cells farthest from a capillary and would likely occur one cell at a time. Diffuse apoptosis or necrosis would be difficult to observe, yet might explain the chronic diffuse inflammation also observed in many of these same degenerative diseases.

It should be recognized that ATP demands are not constant, that ATP demand fluctuates with the metabolic load on a cell due to all cellular functions. Obviously problems of insufficient ATP only result if demand exceeds supply. ATP levels are under feedback control. A mismatch in ATP demand and supply can occur with a small disruption within the feedback system (i.e. nitropenia), or with a gross disruption outside the feedback system (i.e. ischemia or hypoxia or mitochondria inhibition).

ATP production is “robust”. The ATP production systems can tolerate some amount of disruption and still maintain ATP levels in the physiologic range. However, at some level of disruption, ATP production would be compromised, and with insufficient ATP, the various “housekeeping” functions of the cell are compromised, which would degrade all cell processes, including ATP production. Which processes would degrade “first”, is unknown, and is likely dependant on idiosyncratic details of individual cell metabolism, local O2 and glucose supply, local metabolic demand, local mitochondria density, and details of DNA expression. Different mitochondrial proteins are expressed in different organs, which because of different metabolic demands, must have different ATP regulation pathways.

It should be recognized that ATP demands are not constant, that ATP demand fluctuates with the metabolic load on a cell due to all cellular functions. Obviously problems of insufficient ATP only result if demand exceeds supply. ATP levels are under feedback control. A mismatch in ATP demand and supply can occur with a small disruption within the feedback system (i.e. nitropenia), or with a gross disruption outside the feedback system (i.e. ischemia or hypoxia or mitochondria inhibition).

Production and regulation of ATP production and consumption is not simple. Because the many pathways are non-linear and are coupled and are not fully understood, their modeling and analysis is difficult. My objective is not to exhaustively explain all pathways, but simply to point out a number of pathways that are NO mediated, and which would be down-regulated by a state of nitropenia and which would then cause a lower ATP production rate. Because the various metabolic processes involving ATP depletion and nitric oxide are non linear and “coupled”, they do not occur in a linear fashion, either in time, or in space, this paper describing them isn't arranged in a linear fashion either. Rather it is arranged in little vignettes discussing various consequences of nitropenia and how some of those consequences exacerbate ATP depletion and how ATP depletion exacerbates many of these conditions.

ATP production comprises a number of sequential and parallel pathways, each of which requires a driving force, and so trades incremental “non-reversibility” for incremental kinetics. Because ATP production pathways have evolved over long periods of time, the various pathways have become “optimized”. What I mean by this is that in general, the various “inefficiencies” in the pathway are distributed over the entire pathway, so as to minimize the total inefficiency. What this means is that there is no one “controlling” pathway that limits ATP production, but rather that the “capacity” of each step in the metabolic pathway is (approximately) matched to the “capacity” of every other step. Excess capacity in any one step is effectively “wasted”, and what ever resources are devoted to that excess capacity would be better spent on other steps that are not present in excess.

It may be that a number of seemingly disparate disorders, characterized by ATP depletion and eventual organ failure are actually “caused” by nitropenia, caused by a global deficiency in basal nitric oxide. When this occurs in the heart, the result is dilative cardiomyopathy. When this occurs in the brain, the result is white matter hyperintensity, Alzheimer's, vascular depression, vascular dementia, Parkinson's, and the Lewy body dementias. When this occurs in the kidney, the result is end stage renal disease, when this occurs in the liver, the result is primary biliary cirrhosis. When this occurs in muscle, the consequence is fibromyaligia, Gulf War Syndrome, or chronic fatigue syndrome. When this occurs in the bowel, the consequence is ischemic bowel disease. When this occurs in the pancreas, the consequence is first type 2 diabetes, followed by chronic inflammation of the pancreas, followed by autoimmune attack of the pancreas (or pancreatic cancer), followed by type 1 diabetes. When this occurs in the connective tissue, the consequence is systemic sclerosis.

While ATP depletion will eventually affect every metabolic process, I will focus on the processes that are known to be disrupted in the major degenerative disorders which I hypothesize are caused by nitropenia. It should be noted that there is positive feedback. Once the cell's ATP production has been compromised and damage starts occurring, that damage will accumulate and ATP production will be further compromised. As the cells “machinery” is damaged, the rate of damage accelerates.

ATP from Oxidative Phosphorylation

Mammalian cells are aerobic. Organic compounds (primarily glucose and fatty acids) are conveyed via the blood stream, actively ported to cells, broken into small bits, fed into the citric acid cycle, oxidized to CO2 and water in the mitochondria, producing reducing equivalents and ATP. To accomplish this, mitochondria must be supplied with organic compounds and O2. O2 is absorbed in the lung, transferred to hemoglobin in erythrocytes, carried by the blood stream, where it diffuses from the terminal capillaries to the mitochondria. The transport of O2 is a purely passive diffusion down a concentration (actually chemical potential) gradient. There is no “active” O2 transport. The chemical potential of O2 (often measured as a partial pressure) at the mitochondria may be at the lowest point in the body because it is at the mitochondria where the O2 is consumed.

Many organs have a variable metabolic rate. For example, the metabolic rate of the heart can vary by nearly an order of magnitude. The geometry of the vasculature does not change appreciably during this change (although there is some increased recruitment of blood vessels). With a constant O2 partial pressure in the blood, and a constant mass transfer area, and a constant diffusion length, the only way 10 times more O2 can be delivered to the mitochondria, is if the concentration gradient increases. The only way for the concentration gradient to increase is for the O2 level at the mitochondria to go down because the level in the capillary is nearly constant and is fixed by the O2 content of the atmosphere. If the mitochondria O2 level goes down an order of magnitude, and the mitochondria O2 consumption goes up an order of magnitude, the specific O2 consumption (O2 consumed per cytochrome oxidase per Torr O2) must go up 2 orders of magnitude. Under basal conditions, O2 consumption occurs at cytochrome oxidase and is inhibited by nitric oxide (NO). To remove the NO inhibition, the NO must be removed. One way to accomplish this is to generate superoxide, which reacts with NO at diffusion limited rates. Thus, one way to accelerate metabolism is to generate superoxide, which destroys NO, disinhibits cytochrome oxidase, the mitochondria now consume O2 at a higher rate, the O2 level local to the mitochondria drops, the concentration gradient of O2 from the vessel to the mitochondria increases, and more O2 can diffuse to the now more active mitochondria. Thus generation of superoxide is seen to be a “feature” that increases local metabolic rate by disinhibiting cytochrome oxidase. However, this only works if the cytochrome oxidase is inhibited by NO. If cytochrome oxidase is not inhibited by NO (i.e. under conditions of nitropenia), adding superoxide does not increase metabolism, it simply causes oxidative damage.

Production of reactive oxygen species (ROS) is observed in hypoxia and in reperfusion, and is a major cause of the damage done by ischemia and hypoxia. A little ROS might be good, if it increased O2 availability by increasing O2 diffusion, but this can only occur when there is sufficient NO present.

The “O2 diffusion resistance” (or some parameter proportional to O2 diffusion resistance) may be measured to determine how the normal capillary spacing and hence the normal diffusion resistance of O2 is set. Hypoxia inducible factor, (HIF-1α) is turned on by “hypoxia”, and causes the transcription of a number of genes that turn on angiogenic factors including VEGF. Sandau et al. have reported to HIF-1α is turned on by the combined signal of high NO and low O2. (Accumulation of HIF-1α under the influence of nitric oxide. Blood. 2001; 97: 1009-1015.)

While the body must initiate angiogenesis when there is insufficient vascular supply, (which might be measured by O2 levels), it must also ablate capillaries when there is “too much” vascular supply. Ablation of capillaries cannot be mediated simply by an “adequate” O2 supply. In organs like the heart, the normal O2 consumption is much lower than the peak consumption. Since “normal” capillary spacing is determined under “normal” conditions, it may be that “hypoxic” sensing is not achieved simply by “low O2 levels”, but may be determined in part by basal NO level, specifically by high NO levels, or more particularly, by the ratio of NO to O2.

Oxygenated hemoglobin (O2Hb) destroys NO at near diffusion limited rates. O2Hb is located in the blood stream and delivers O2 to mitochondria. All mitochondria must necessarily be diffusively close to O2Hb so as to receive O₂ for oxidative phosphorylation. With O2Hb also being the sink of NO, the minimum NO level must also be at the site of O2Hb. Thus in the extravascular space, the vessel wall is the NO minimum, and the NO concentration is a measure of “how far” a cell is from O2Hb, exactly the measure that is needed to determine O2 diffusion resistance. The ratio of NO/O2 would thus be an excellent measure of when a particular site needs more (or less) O2 exchange capacity. A number of physiological responses to “not enough O2”, are mediated through HIF-1α. HIF-1α is regulated in part by NO, where a higher NO level increases the O2 level at which HIF-1α is turned on.

Nitropenia may have an effect on the spatial distribution of HIF-1α as a function of O2 level. With a lower NO level, lower O2 levels will be required to turn on HIF-1α. Thus as capillaries remodel (which they do continuously), they will gradually become farther apart until the O2 level drops low enough for the NO/O2 ratio to trigger HIF-1α at the point farthest from a capillary. The “normal” capillary spacing is determined during “normal” physiological conditions. A slightly lower O2 level might be tolerable under basal conditions, but inadequate under higher metabolic load.

With a lower NO level, lower O2 levels will be required to turn on HIF-1α. Thus as capillaries remodel (which they do continuously), they will gradually become farther apart until the O2 level drops low enough for the NO/O2 ratio to trigger HIF-1α at the point farthest from a capillary. The “normal” capillary spacing is determined during “normal” physiological conditions. A slightly lower O2 level might be tolerable under basal conditions, but inadequate under higher metabolic load.

There are no reports of NO gradients between capillaries, and few reports of O2 gradients. However, when people do not exercise regularly, they go “out of shape.” Their capacity for aerobic metabolic activity is reduced. This indicates that vascular remodeling does ablate capillaries so as to reduce O2 diffusive capacity. The time scale for changes in aerobic capacity indicates the time scale at which this vascular remodeling occurs. Low NO levels would modify the level of aerobic exercise necessary to effect physical conditioning. With high NO levels, modest exercise might produce significant aerobic capacity. With lower NO levels, greater levels of exercise producing greater metabolic hypoxia would be required. While increased metabolic activity can be induced periodically in muscle through exercise, the metabolic demand of some organs does not fluctuate the way muscle does.

Thus capillary rarefaction would reduce the maximum metabolic capacity of the tissue served by that capillary bed. Under basal conditions, the reduced maximum capacity might not be apparent, under conditions of nitropenia, in large part because with low NO, the O2 level at the mitochondria is lower too, and O2 diffusion to meet basal demands can be accommodated through rarefacted capillaries because of the increased O2 gradient. However, under conditions of increased metabolic load, metabolic capacity might be insufficient to meet metabolic demand and conditions of ATP depletion would occur.

Each organ has different metabolic functions, and different circumstances that increase metabolic load. For example, in the kidney, a major metabolic load is resorption of sodium. Increased dietary sodium will then increase the metabolic load on the kidney and if the metabolic capacity is exceeded, will cause ATP depletion and dysfunction. In dilative cardiomyopathy, the heart becomes more sensitive to hypoxia and to overload. In fact, in animals, dilative cardiomyopathy can be induced simply by chronic heart overload, either through pacing, or through pressure overload. This is consistent with the hypothesis of NO mediated capillary rarefaction. When the heart is overloaded, there is insufficient O2 delivered to the heart muscle. Superoxide is generated to destroy NO, disinhibit cytochrome oxidase, and drop O2 concentration so that more O2 can diffuse to the overloaded muscle. Acutely, this increases metabolic capacity (but only when cytochrome oxidase is inhibited by NO). However, chronic low NO causes vascular remodeling and the capillary rarefaction that is characteristic of dilative cardiomyopathy. The superoxide damages proteins, the low ATP level reduces the rate of ubiquinated protein disposal by the proteosome, and hyperubiquinated proteins accumulate.

Similarly, in the remnant kidney model of end stage renal disease, part of the kidney is removed, (either surgically or with a toxin) which increases the metabolic load on the remainder. Superoxide is generated to decrease NO and increase O2 diffusion to the kidney mitochondria. Chronic overload results in progressive kidney capillary rarefaction and progressive kidney failure. In acute kidney failure, putting people in dialysis can give the kidney a “rest”, and allows it to recover. In acute renal failure induced by rhabdomyolysis (muscle damage which releases myoglobin into the blood stream) kidney damage is characterized by ischemic damage. Myoglobin scavenges NO, just as hemoglobin does, and would cause vasoconstriction in the kidney leading to ischemia. Myoglobin would also induce local nitropenia and the cascade of events leading to further ATP depletion.

Lowering metabolic load can allow the kidney time to recover, but if there is a low basal level of NO, the kidney vasculature would remain rarefacted and the kidney would remain very susceptible to metabolic overload.

Increased capillary spacing increases the diffusion resistance for O2, which is in part compensated by reduced inhibition of cytochrome oxidase by NO, leading to a lower O2 concentration at the mitochondria. Transport capacity of glucose is also reduced. O2 is carried by erythrocytes, which remain confined to the vasculature. In contrast, glucose is dissolved in the plasma, and plasma permeates the extravascular space and is actively ported into cells via numerous types of glucose transporters. Unfortunately, measurement of extravascular glucose is difficult and there are few measurements reported in the literature. However, it must be lower than blood sugar, because glucose is consumed as extravascular fluid permeates the extravascular space. Because glucose is consumed, there must be gradients in glucose concentration, just as there are gradients in O2 concentration. Transport of O2 is by diffusion, transport of glucose is by diffusion, convection and by active transport. Presumably, capillary rarefaction would result in lower glucose concentrations because more cells are consuming the glucose supplied by a given capillary. In contrast to O2 concentration, glucose concentration can be increased to provide a larger concentration gradient. Similarly, the concentration of glucose transporters can also be increased. It is perhaps possible that the increased blood sugar observed in type 2 diabetes is compensatory, so as to increase delivery of glucose to tissues too far from a capillary. Similarly, the increased insulin release may be compensatory so as to increase the concentration of glucose transporters.

The main source of ATP is oxidative phosphorylation. Cells can derive ATP through glycolysis, however, glycolysis consumes 19 times more glucose per unit of ATP than does oxidative phosphorylation. If capillary rarefaction proceeds to the point where O2 supplies are compromised, and the cell must derive ATP from glycolysis, glucose consumption would increase greatly. If glucose consumption exceeded supply, ATP depletion would invariably occur.

Appetite is regulated in part through measurement of glucose concentration. Presumably, this measurement does not occur precisely in the large vessels where glucose is most constant, but in peripheral tissues, in the extravascular space. If the cells which sense glucose and so regulate appetite are in between rarefacted capillaries, they might register a low glucose level in spite of the bulk glucose content of the blood being adequate. In the presence of rarefacted capillaries, “normal” blood sugar may register as too low, and the body might respond with hyperglycemia. If capillary rarefaction is sufficient to impair oxidative phosphorylation, glycolysis may be insufficient to maintain ATP supplies despite elevated blood sugar and elevated insulin levels. If cells in a rarefacted capillary bed experienced low glucose and/or low ATP levels, they might send the signal “I am starving” to the brain and increase appetite. People with rarefacted capillaries may continue to eat, despite adequate reserves of body fat, because the cells that sense glucose homeostasis don't have enough. The carbohydrate craving, elevated blood sugar, insulin resistance and dysregulated appetite of obesity may be a consequence of the rarefacted capillaries which are observed in obesity.

Mitochondria biogenesis is initiated by cGMP from guanylyl cyclase either through an increase in NO at constant ATP, or a drop in ATP at constant NO. A reduced basal NO level will therefore reduce the concentration of mitochondria and will decrease the basal ATP concentration. The efficiency of oxidative phosphorylation decreases as the rate (mL O₂/mg protein) increases. The rate of ATP production depends on the mitochondria potential with a high ATP production rate at a high ratio of ATP/ADP requiring a high mitochondrial potential.

A number of the symptoms of the metabolic syndrome may be exacerbated by ATP depletion due to mitochondria depletion caused by nitropenia. With mitochondria depletion there is increased generation of ATP via glycolysis. However because glycolysis produces 1/19 as much ATP, greater blood glucose is required. Glucose import in cells is limited by glucose transporters, which are induced by insulin. Most cells are not in direct contact with blood, but are in the extravascular space where they are perfused by plasma, and where the glucose and insulin concentrations are less than in the blood due to consumption by intervening cells. Capillary spacing appropriate for glucose delivery to produce ATP via oxidative phosphorylation will be woefully inadequate to produce the same ATP via glycolysis. Cells “too far” from a capillary might have local inadequate glucose even under conditions of hyperglycemia in bulk blood. Such ATP depleted cells might send the signal “I am starving”. Such starvation signals might compel consumption of carbohydrate despite adequate or even surplus whole organism reserves of depot fat.

Mitochondria Biogenesis/Regulation

The critical “engine” of ATP production is the mitochondria. All multi-cellular organisms have mitochondria, as do some single celled organisms. The mitochondria content of tissues is variable, with heart muscle approaching 20-30% by volume, compared to a few % in less aerobic muscles. Mitochondria are the site of much ROS generation, and some components of mitochondria are sensitive to irreversible damage and when mitochondrial components become inoperative, they must be replaced. Because different cells have different mitochondria densities, presumably there are mechanism(s) that regulate the different densities in the various cells. Presumably this includes mechanism(s) for increasing mitochondria number when too low, and for ablating mitochondria when too high.

Mitochondria biogenesis has been shown by Nisoli et al. to be initiated by NO via soluble guanylyl cyclase (sGC) via cGMP. (Nisolie, et al., Mitochondrial biogenesis in mammals: The role of endogenous nitric oxide, Science, Feb. 7, 2003, Vol 299, 896-899.) sGC has been shown by Ruiz-Stewart et al. to be sensitive to both NO and ATP levels, where the threshold for NO triggering of cGMP production is proportional to ATP level, that is, at a lower ATP level, sGC is more sensitive to NO, and vice versa. (Ruiz Stewart et al., Guanylyl cyclase is an ATP sensor coupling nitric oxide signaling to cell metabolism, PNAS Jan. 6, 2004, Vol 101, No. 1, 37-42.) At constant NO levels, falling ATP will trigger sGC and produce cGMP. However, at low basal NO levels (nitropenia) the ATP level which triggers cGMP production will be lower than at high NO levels. Thus mitochondria biogenesis will be lower under conditions of nitropenia. With fewer mitochondria, each mitochondria will be working at a higher O2/substrate turnover rate.

It is necessary for peak metabolic capacity to exceed “normal” metabolic capacity “at rest”. Presumably, this difference arises from the production of “excess” mitochondria, that is more mitochondria than are needed to supply basal metabolism. Presumably, if ATP is the signal for mitochondria biogenesis, there must be mitochondria inhibition under basal conditions to allow for excess mitochondria production at basal ATP concentrations. That inhibition is then released during peak metabolic capacity allowing for increased ATP production. NO fills the role of the inhibitor. NO inhibits cytochrome oxidase. Reduction in NO accelerates metabolism.

Nogueria et al. has reported that, in general, the efficiency of oxidative phosphorylation decreases as the rate (mL O₂/mg protein) increases. (Nogueria et al., Mitochondrial respiratory chain adjustment to cellular energy demand, J. Biol Chem 276, 49, 46104-46110, 2001.) Also, Kadenbach has reported that the rate of production of ATP depends on the mitochondria potential with a high ATP production rate at a high ratio of ATP/ADP requiring a high mitochondrial potential. (Kadenbach, Intrinsic and extrinsic uncoupling of oxidative phosphorylation, Biochimica et Biophysica Acta 1604 (2003) 77-94.)

Mitochondria are major producers of ROS. The production of ROS by mitochondria is strongly dependent on the mitochondria potential, with higher potential exponentially increasing ROS generation.

When the density of mitochondria is lower, each mitochondria will be working “harder”, operating at a higher potential, producing more ROS and producing ATP with a lower efficiency. With higher ROS generation, mitochondrial protein damage is expected to be greater. High mitochondrial potential and high ROS generation cause induction of uncoupling proteins as reported by Echtay et al. ( Echtay et al., Superoxide activates mitochondrial uncoupling protein 2 from the matrix side, J Biol Chem 277, 49, 47129-47135, 2002). This serves to reduce the mitochondrial potential and reduce ROS generation as reported by Sluse et al. (Uncoupling proteins outside the animal and plant kingdoms: functional and evolutionary aspects. FEBS Letters 510 (2002) 117-120.) Uncoupling protein 2 is abundantly expressed in primary biliary cirrhosis and is reduced following successful treatment with ursodeoxycholic acid (which decreases liver metabolic load by displacing bile synthesis) as reported Taniguchi et al. (Taniguchi et al., Expression of uncoupling protin-2 in biliary epithelial cells in primary biliary cirrhosis, Liver 2002: 22: 451-458.)

The consumption of O₂ by cytochrome oxidase is inhibited by NO. Under basal-conditions, cytochrome oxidase is mostly inhibited, and consumption of O2 occurs at a high O2 partial pressure. The consumption of O₂ at the mitochondria produces the O₂ concentration gradient which drives the purely passive O₂ diffusion to the mitochondria. At higher levels of oxidative phosphorylation, O₂ consumption can increase ˜10×, however, the path length for diffusion of O₂ is not greatly altered, and neither is the O₂ concentration at the vessel wall. To increase the O₂ consumption of heart muscle ˜10× at constant diffusion geometry, the O₂ gradient must increase ˜10× and the terminal O₂ concentration must drop ˜ 1/10. This change in the affinity of cytochrome oxidase for O2 is accomplished in part by changing the NO concentration. By lowering the NO concentration, the affinity of mitochondria for O₂ is increased, and the ATP production per mitochondria is increased, albeit at a reduced efficiency and increased ROS generation. The superoxide that accompanies higher O₂ consumption lowers NO levels and allows high O₂ consumption at low O₂ concentration which allows for high O₂ diffusion to the mitochondria. Thus the production of superoxide at high ATP production rate is a “feature” which facilitates high O₂ consumption by consuming NO.

It may be that cellular demand for ATP is not reduced despite decreased mitochondria density. Producing the same ATP at a reduced mitochondria density will result in an increase in O2 consumption, or an accelerated basal metabolic rate. An accelerated basal metabolic rate is observed in a number of conditions, including: Sickle cell anemia, Congestive heart failure, Diabetes, Liver Cirrhosis, Crohn's disease, Amyotrophic lateral sclerosis, Obesity, End stage renal disease, Alzheimer's, and Chronic obstructive pulmonary disease.

While some increased O2 consumption might be productively used, in many of these conditions uncoupling protein is also upregulated, indicating that at least part of the increased metabolic rate is due to inefficiency. Conditions where uncoupling protein is known to be upregulated are: Obesity and Diabetes.

It may be that conditions that increase ROS would cause the induction of UCP2, which would have the effect of reducing ATP levels further. Superoxide destroys NO, and reduces NO levels still further. Thus nitropenia sufficient to reduce mitochondria biogenesis will result in ATP depletion, which will lead to greater mitochondria ROS generation which will lead to further NO reduction and still lower mitochondria biogenesis. Nitropenia will lead to end stage degenerative diseases characterized by ATP depletion, ROS generation, UCP induction, mitochondria ablation, and eventual organ failure.

Thus, nitropenia will result in fewer mitochondria which can produce the same ATP but with lower efficiency, with lower “reserve” metabolic capacity, at lower O₂ concentration at the mitochondria, and with greater superoxide production.

With fewer mitochondria consuming O₂ to a lower O₂ concentration, the O₂ gradient driving O₂ diffusion is greater, so the O₂ diffusion path length can increase resulting in capillary rarefaction, which is observed in dilative cardiomyopathy, hypertension, diabetes type 2, renal hypertension.

Hypoxia Inducible Factor HIF-1α

Many of the effects of “hypoxia” are mediated through hypoxia-inducible factor (HIF-1α) which activates transcription of dozens of genes including the EPO gene. Complex behavior of HIF-1α in response to NO exposure has been demonstrated using authentic NO, NO donors and also transfected cells expressing iNOS as NO sources as reported by Sandau et al. (Sandau et al., Accumulation of HIF-1α under the influence of nitric oxide. Blood. 2001;97:1009-1015.) Sandau et al. found that lower NO levels induced a more rapid response and produced more HIF-1α than did higher levels. The only NO donor tested which did not induce HIF-1α was sodium nitroprusside which also releases cyanide. Because HIF-1α senses both high NO and low O2, with low NO, a lower O2 level is required to turn HIF-1α on. A number of pathways require HIF-1α induction, including anaerobic glycolysis, which can produce ATP under anaerobic conditions from glucose and produce lactate, glucose transporters which port glucose into the cell, VEGF which is part of the angiogenesis pathway, and erythropoietin which triggers the production of erythrocytes and raises hematocrit.

Goda et al. have reported that HIF-1α is also necessary for arrest of the cell cycle via p53. (Goda et al., Hypoxia-Inducible Factor 1α is essential for cell cycle arrest during hypoxia, Molecular and cellular biology, January 2003, p 359-369.) Arrest of the cell cycle is important under conditions of hypoxic stress, so that cell division does not occur under conditions of insufficient ATP, which leads

Thus a reduced basal NO level would result in reduced expression of HIF-1α mediated genes, and lower levels of glucose transporters (causing glucose “resistance”), reduced levels of Epo (causing anemia),

Estimate of NO Absorption on Skin from AAOB

The motivation for this analysis is to estimate the bioavailability of NO produced by AAOB and absorbed through the skin. The main difference between the lung and the skin as exchange surfaces for gases has to do with the proximity of hemoglobin. In the lung, efficient O2 loading is required and arterial blood leaving the lung is typically >90% saturated with O2. Oxygenated Hb destroys NO very rapidly. Deoxygenated Hb also binds NO rapidly, rendering it unavailable. In contrast to the reactions with Hb, the reactions with albumin preserve the vasodilatory activity of NO through the formation of a variety of NO containing species, including S—NO-albumin, as NO physically adsorbed in hydrophobic regions of the albumin molecule as reported by Sampath et al. (Sampath et al., Anesthetic-like Interactions of Nitric Oxide with Albumin and Hemeproteins, A Mechanism For Control Of Protein Function, The Journal Of Biological Chemistry Vol. 276, No. 17, Issue of April 27, pp. 13635.13643, 2001.) There is also formation of a nitrosating species reported by Nedospasov et al. (Nedospasove et al., An autocatalytic mechanism of protein nitrosylation, PNAS, Dec. 5, 2000, vol. 97, no. 25, 13543-13548.) The nitrosating species is reported by Rafikova et al to be N2O3 also adsorbed in hydrophobic regions. ( Rafikova et al., Catalysis of S-nitrosothiols formation by serum albumin: The mechanism and implication in vascular control, PNAS Apr. 30, 2002, vol. 99, no. 9, 5913-5918.) This last reference demonstrates that albumin can promptly react with authentic NO and O2 to form complexes that are stable for minutes and which slowly release authentic NO, and that these NO—O2-albumin complexes cause vasodilatation in vivo on rats vasoconstricted with L-NAME. These complexes also cause the nitrosation of diverse materials including low molecular weight thiols. In vitro, blocking the sulfhydryl groups prevented formation of S—NO-albumin, but did not prevent the formation of this NO—O2-albumin nitrosating complex. S—NO-albumin also transnitrosates glutathione, especially in the presence of Cu containing proteins such as ceruloplasmin. S—NO-thiols also release NO, and it is not clear exactly which species, NO, GSNO, other low molecular weight S—NO-thiols or S—NO-albumin are important active species, but perhaps all of them are.

According to one aspect of the invention, it is appreciated that the transport mechanism for moving NO species from the skin to guanylyl cyclase (GC) where it can act is via S—NO-thiols, either S—NO-albumin, GSNO, or other low molecular weight species. The advantages of using the skin as the exchange surface for nitrosylation of albumin are several. First, it would allow the NO to be absorbed into the extravascular plasma substantially without encountering Hb. The lifetime of NO species in plasma without Hb is very long. Second, the external skin is much more tolerant of NOx than is the lung. The outer surface is actually dead, and is continually renewed. If the NO-albumin complexes formed in vitro are the species which transport NO systemically in vivo, then the therapeutic effectiveness of transdermal NO would be many-fold higher than that through inhalation. Third, since the expected active species is an S—NO-thiol, the non-enzymatic oxidation of NO with O2 does not destroy NO, it converts it to N2O3 which is a good nitrosating agent.

Autotrophic ammonia oxidizing bacteria may be commensal, and humans may have evolved to utilize the NO that they produce, so there should not be any deleterious side effects from their use to raise basal NO levels. According to one aspect of the invention, it is appreciated that many of the diseases of the modern world result from an NO deficiency due to the loss of these bacteria through modern bathing practices. Positive side effects, particularly in those of recent African decent whose recent ancestors didn't evolve compensatory NO pathways to deal with the loss of NO from AAOB during winter may result from use of AAOB. This may be one reason why the African American community is hit harder by obesity, diabetes, hypertension, asthma, atherosclerosis, heart disease, end stage renal disease, precocious puberty, etc. Photochemical dissociation of NO from SNO-thiols is well known, and the loss of skin and hair pigmentation at high latitudes may derive from a need for increased photochemical dissociation of SNO-thiols in the external skin and not from vitamin D metabolism. Sweating on the scalp increases at night, when photo dissociation of SNO-thiols would be at a minimum. Hair becomes white with age, perhaps to allow greater light penetration for photochemical NO release. Tyrosinase, the enzyme that forms melanin is a type-3 copper containing oxidase, a number of which catalyze the formation of SNO-thiols.

The external skin derives all of its metabolic O2 needs from the external air. There is thus no need for erythrocytes to circulate through those regions, and for the most part, they does not. For the most part the color of skin is due to pigment and erythrocytes. Non pigmented skin is relatively transparent, and the color accurately reflects the circulation of erythrocytes in the surface layers. While the living outer layers of skin derive O2 from the atmosphere, they derive all other nutrients from the blood. Plasma is blood without erythrocytes, and thus can supply everything except O2. Since the outer layers of skin are essentially erythrocyte free, but are still actively metabolizing, plasma may be circulating through those outer layers of skin which derive O2 from the atmosphere. It is in this erythrocyte free skin that conversion of NO to S—NO-albumin occurs.

The lifetime of NO in the blood is extremely short. NO is rapidly oxidized by O2Hb, rapidly binds to Hb, is complexed by albumin, is oxidized to N2O3 and NO2 through non-enzymatic reaction with O2, and also forms S—NO-thiols. Bellamy et al. reported that a significant site of action of NO is guanylyl cyclase (GC) where the apparent EC50 is about 45 nM/L for rapid (˜100 ms) and 20 nM /L for slow (˜1 to 10 sec) activation. (Bellamy et al., Sub-second Kinetics of the Nitric Oxide Receptor, Soluble Guanylyl Cyclase, in Intact Cerebellar Cells, The Journal Of Biological Chemistry Vol. 276, No. 6, Issue of February 9, pp. 4287-4292, 2001.) There are significant difficulties in estimating the fraction of an administered dose of an NO source that will reach the target tissues in pharmacological amounts. For example, when inhaled NO is administered at 80 ppm in >90% O2 (16 μM/min=14 μM/kg/hr) there is no change in mean arterial pressure. In contrast, Cockrill et al. reported that sodium nitroprusside (SNP) at 0.9 μM/min (0.75 μM/kg/hr) causes a 25% reduction in mean arterial pressure. (Cokrill et al., Comparison of the Effects of Nitric Oxide, Nitroprusside, and Nifedipine on Hemodynamics and Right Ventricular Contractility in Patients With Chronic Pulmonary Hypertension* CHEST 2001; 119:128-136.) This may indicate that when administering NO through inhalation, the concentration of NO at the resistance determining vessels does not increase to 20 nM/L and activate GC. Thus SNP is many times more “effective” at delivering “NO active species” to peripheral GC than is inhaled NO.

SNP has also been compared to intravenous NO, where intravenous NO, SNP, and S—NO-glutathione (GSNO) were shown by Rassaf et al. to have relative “maximally effective doses” administered as bolus infusions in local brachial artery vasodilatation of 6 μM, 34 nM, and 5 nM respectively. (Rassaf et al., Evidence for in vivo transport of bioactive nitric oxide in human plasma, J. Clin. Invest. 109:1241-1248 (2002).) This puts the relative effectiveness of intravenous NO, SNP, and GSNO at 1:176:1200. There were significant differences in the temporal course of vasodilatation induced through the above treatments. Both the NO and the GSNO treatments had a more sustained effect than SNP. Thus GSNO is roughly 7 times more “effective” at getting “NO active species” to peripheral GC than is SNP. Presumably then, a dose of about 0.1 μM/kg/hr of GSNO would have a vasodilatation effect equivalent to 0.75 μM/kg/hr SNP. The basal nitrate excretion is about 1 μM/kg/hr. If we assume that the vasodilatory effects of 0.75 μ/kg/hr SNP are on the “same order” as the indigenous NO already produced, then the 0.1 μkg/hr GSNO represents an increase in “effective NO” of 50% over basal levels.

Copper, either as Cu2+ or as ceruloplasmin (CP) (the main Cu containing serum protein which is present at 0.38 g/L in adult sera and which is 0.32% Cu and contains 94% of the serum copper) catalyzes the formation of S—NO-thiols from NO and thiol containing groups (RSH). CP in sub μM/L concentrations had activity greater than that of free Cu2+, and in the presence of physiologic chloride concentrations the activity was approximately doubled. A number of other Cu containing enzymes also catalyze the formation of S—NO—R:

Katsuhisa Inoue et al., demonstrate that copper ions and a number of copper containing enzymes catalyze the formation of S—NO—R compounds, for example they measure the nitrosothiol-producing activities of various copper-containing proteins. (Katsuhisa et al., Nitrosothiol Formation Catalyzed by Ceruloplasmin Implication For Cytoprotective Mechanism In Vivo, The Journal Of Biological Chemistry Vol. 274, No. 38, Issue of September 17, pp. 27069-27075, 1999.) RS—NO was formed in the reaction of reduced glutathione (GSH) (20 μM) or N-acetyl-L-cysteine (NAC) (20 μM) and P-NONOate (10 μM) with or without CuSO4 or various copper containing proteins. CuSO4 or copper-containing proteins (protein subunits) were used at a concentration of 2.0 μM. The amount of RS—NO (GS—NO and NAC—NO) reached a plateau or declined when the concentration of CuSO4 or each copper-containing protein exceeded 2 μM. Data are the means 6 S.E. of four experiments”.

The formation of GSNO from NO and GSH is shown to be approximately 100 times greater in the presence of physiologic concentrations of CP. They also report that CP produced significant GSNO even at nanomolar concentrations of NO.

They also show that in cell culture, murine macrophage cells (RAW264) with iNOS-induced by interferon-γ and lipopolysaceharide, and supplemented with CP (2 μM/L) in Krebs-Ringer-phosphate, roughly ⅓ of the oxidized NO species produced, (nitrate, nitrate and RSNO) ended up as recovered NAC—NO. This finding is remarkable. It demonstrates that in the absence of hemoglobin, conversion of authentic NO to RSNO can be quite efficient and as high as 33%.

The Cu content of plasma is variable and is increased under conditions of infection. Berger et al. reported that the Cu and Zn content of burn-wound exudates is considerable with patients with ⅓ of their skin burned, losing 20 to 40% of normal body Cu and 5 to 10% of Zn content in 7 days. (Berger et al., Cutaneous copper and zinc losses in burns, Burns, October 1992;18(5):373-80.) It may be that the Cu in burn exudates is there to catalyze the conversion of NO into S—NO-thiols. As an aside, if the patients skin were colonized by AAOB, wound exudates which contains urea and Fe, Cu, and Zn that AAOB need, would be converted into NO and nitrite, greatly supplementing the local production of NO by iNOS, without consuming resources (such as O₂ and L-arginine) in the metabolically challenged wound. A high production of NO and nitrite by AAOB on the surface of a wound would be expected to inhibit infection, especially by anaerobic bacteria such as the Clostridia which cause tetanus, gas gangrene, and botulism. The xanthine oxidase content of the skin would increase NO levels by reducing any nitrite produced by the AAOB into NO. Inhibiting the Clostridia which cause botulism food poisoning is the primary reason for the use of nitric oxide (as nitrite) to cure and preserve meat. In a textbook on microbial disease, the author of the chapter on Clostridia, Rubin writes: “In some developing countries the umbilical stump of newborn children is packed with mud or dung to soothe the infant.” (E. Rubin, The Clostridia chapter 11 in Mechanisms of Microbial Disease ed. M. Schaechter, G. Medoff, D. Schlessinge, Williams & Wilkins, 1989, Baltimore Md.) Rubin suggests that such a procedure prevents tetanus infection by rendering the wound aerobic however, the actual anti-tetanus agent may be nitric oxide produced by the AAOB bacteria in mud when acting on the ammonia and urea found in dung.

The skin contains 9.2 ppm Fe, while whole blood contains 500 ppm Fe and plasma contains 1 ppm Fe. The major concentration of hemes in the skin is hemoglobin in the capillaries, which is why the color of skin reflects perfusion. Since the heme content of the skin is at most 2% that of the blood, it would be expected that in the skin, NO would have a lifetime at least 50 times that in the blood. Actually it would be more, because some of the iron is present not as hemes, but as iron complexes that are not reactive toward NO. The skin represents 18% of adult body weight and contains 23% of the body's albumin (about 65 g for 70 kg male). NO reacts with O2Hb to form nitrite and nitrate which are inactive. NO reacts with thiols to form S—NO-thiols, and has a non-enzymatic reaction with O2 to form NO2. NO2 can readily nitrosate thiols too. The non-enzymatic reaction with O2 thus does not remove and prevent NO from forming S—NO-thiols. A reaction in determining the production of S—NO-albumin in the skin is the destruction of NO by O2Hb. All of the NO that is not so destroyed should instead form S—NO-albumin. Actually, Godber et al. reported that NO that is converted into nitrite or nitrate can be reduced into NO by xanthine oxidoreductase. (Gobert et al., Reduction of Nitrite to Nitric Oxide Catalyzed by Xanthine Oxidoreductase, The Journal Of Biological Chemistry. Vol. 275, No. 11, Issue of March 17, pp. 7757-7763, 2000.) Similarly, nitrite and nitrate can be excreted by sweat ducts and then “recycled” by the AAOB, which can use nitrite or nitrate instead of O2 under anaerobic conditions.

The O2 permeability of the stratum corneum of the skin is about 3.7E-7 ml/m/min/mmHg and 1.3 E-6 in the living portion. The stratum corneum is about 10 to 20 microns thick. The viable epidermis and the stratum papillare extend to about 250 microns, and both are supplied with O2 from the external atmosphere and not from the vasculature. The permeability of both tissues increases as the water content increases. The hydration state of the stratum corneum was not specified, so a higher permeability might be expected on a sweating scalp.

The physical properties of O2 and NO are quite similar, including the partitioning between aqueous and lipid phases, so the permeability of skin to NO is similar to that of O2, however, NO is a lighter molecule which has greater solubility in water and other fluids. If we assume the permeabilities vary as does the solubility in water, then NO would have a 1.5 greater permeability than O2. If the internal NO concentration exceeded 20 nM/L, then GC would be activated, the local vessels would dilate, blood flow would increase, and the NO in excess of 20 nM/L would be convected away or oxidized by O2Hb. 20 nM/L corresponds to a gas phase concentration of 10 ppm. The NO flux through the skin would then be proportional to the concentration difference, the permeability of the skin, and the thickness of the various layers.

The main unknowns are the thickness of skin that the NO must diffuse through to reach the plasma where it is converted into RSNO species. The glutathione (GSH) content of the stratum corneum of hairless mice is about 100 pM/μg protein, or about 0.3%. The second unknown is the efficiency of conversion of NO to RSNO.

The diffusion resistance of an external “biofilm” would be easy to adjust therapeutically. Any gel forming material such as KY jelly or various hair gels would present a diffusion barrier to NO loss through the hair to ambient air. The NO level in the skin cannot greatly exceed 20 nM/L because that level activates GC and would cause local vasodilatation and oxidative destruction of excess NO. The NO concentration at the stratum corneum will increase until it either diffuses away, or the bacteria producing it are inhibited. Which will happen first depends primarily on the external resistance which is easily adjusted.

The scalp can be modeled as a bioreactor generating NO from injected sweat. However, the only loss mechanisms from the scalp biofilm are diffusion through the scalp and diffusion to the ambient air. The biofilm can be thought of as a reactor cycling between dry aerobic and wet anaerobic conditions. NH3 would be oxidized to nitrite which would accumulate as dry solid. Urea would hydrolyze to ammonia and would raise the pH to 7 to 8. AAOB are very active at this pH range and would lower the pH to about 6 where the NH3 converts to ammonium and is unavailable. Metabolism would be inhibited by low water activity as the scalp dried out. Under periods of intense sweating, the pores would be flooded with fresh sweat. Simon et al. disclosed that at pH around 4 where decomposition of nitrite is significant and AAOB can still metabolize urea into nitrite. (Simon et al., Autotrophic Ammonia Oxidation at Low pH through Urea Hydrolysis, Applied And Environmental Microbiology, July 2001, p. 2952-2957.) This fresh sweat would dissolve accumulated nitrite and wick it toward regions of low pH due to the pH dependence of the surface tension of sweat (higher at low pH). The low pH regions are where AAOB are most active and are converting a cation (NH4+) into an anion (NO2-), lowering the pH. As the pores filled with sweat, the bottom of the biofilm would become anaerobic and the AAOB would use nitrite instead of O2. Schmidt et al. reported that under anaerobic conditions (using gaseous NO2 as well as nitrite) the consumption of NH3, NO2 and the production of NO go in the ratio of 1:2:1. (Schmidt et al., Anaerobic Ammonia Oxidation in the Presence of Nitrogen Oxides (NOx) by Two Different Lithotrophs, Applied And Environmental Microbiology, November 2002, p. 5351-5357.) Since the only exit route for nitrogen is as NO, essentially all NH3 and urea excreted is converted to NO. Under these conditions, the average NO production from basal sweating would be about 125 μM/hr based on 0.15 liter sweat/day at 20 mM/liter NH3=3 mM/d at 100% conversion=3 mM/d=125 μM/hr. Others such as Weiner et al. have administered 1 mM NO/hr in inhalation air. (Weiner et al., Preliminary assessment of inhaled nitric oxide for acute vaso-occlusive crisis in pediatric patients with sickle cell disease, JAMA 2003; 289:1136-1142.) The skin also contains xanthine oxidoreductase which rapidly and quantitatively reduces nitrite to NO.

If the pores of the biofilm fill with sweat, the diffusion resistance of a thickness of biofilm to nitric oxide could approach that of the skin. The skin thickness is limited by the diffusion resistance of nutrients from the capillaries to the living cells and so cannot become arbitrarily thick as the bioflim can.

The skin is 3 dimensional, and these bacteria (some of which are motile) may migrate into the sweat ducts where they would have a better supply of urea and ammonia, and where their NO would be absorbed better; The defining characteristic of mammals is the mammary gland, which is a modified sweat duct. All mammals have sweat glands, although many species do not use sweat glands for cooling, including rodents, dogs, and cats. Sweat glands are concentrated on the feet.

Relying on bacteria to produce NO from the urea in naturally excreted sweat allows natural physiological mechanisms to regulate NO administration. Adrenergic mediated sweat on the scalp may occur for exactly that purpose.

### EXAMPLE

The inventor has had AAOB living on his unwashed skin for 27 months now (33 months on the scalp). During that time, his long term essential hypertension declined significantly,, and for a time he did not require medication for its control, he has lost 30 pounds due to a decreased appetite, and without the discomfort that prior weight loss attempts have involved, and liver enzymes have declined into the normal range. He has experienced multiple nocturnal erections virtually every night. Subjectively, he has experienced greater mental acuity and greater tolerance for heat. He and others have noted more vivid dream states.

Method of Use of the Present Invention

According to an aspect of the invention, it is appreciated that many modern degenerative diseases may be caused by a lack of NO species, and that AAOB on the external skin can supply those species by diffusion, and that application of AAOB to the skin resolves long standing medical conditions. In another embodiment of the invention, AAOB are applied to a subject to offset modern bathing practices, especially with anionic detergents remove AAOB from the external skin.

There are a number of different strains of AAOB. However, they are all very similar. They are all autotrophic, so none of them are capable of causing infection. The preferred strain would utilize urea as well as ammonia, so that hydrolysis of the urea in sweat would not be necessary prior to absorption and utilization by the bacteria. Also, in order to grow at low pH, the bacteria must either absorb NH4+ion, or urea. The selected strain should also be capable of living on the external skin, and be tolerant of conditions there. The method I used to isolate such a strain, was to recover a mixed culture from barnyard soil, grow it in organic free media for some months, then apply it to my body, and some months later re-isolate the culture from my body. This selects for strains that are capable of living on the body.

The re-isolated culture is then grown in organic free media, and the active culture is then applied topically. One advantage of using organic free media is that there is no substrate for heterotrophic bacteria to metabolize except for that produced by the autotrophic bacteria. Another advantage of using the as-grown culture is that substantial nitrite accumulates in the culture media, and this nitrite is also inhibitory of heterotrophic bacteria and so acts as a preservative during storage. When the active culture is applied, xanthine oxidase in the skin reduces the nitrite to nitric oxide, creating a “flush” of NO. While this prompt NO is useful, the long term continuous administration of NO is more important.

The ideal method is to apply sufficient bacteria and then wear sufficient clothing so as to induce sweating. However, many people will want to derive the benefits of AAOB while maintaining their current bathing habits, in which case, a culture of the bacteria can be applied along with sufficient substrate for them to produce NO. A nutrient solution approximating the inorganic composition of human sweat is optimal. Using bacteria adapted to media approximating human sweat minimizes the time for them to adapt when applied. Since sweat evaporates once excreted onto the skin surface, using a culture media that has a higher ionic strength is desirable. The inventor has used a concentration approximately twice that of human sweat, but other conditions could work as well.

The strain utilized by the inventor does not utilize urea directly, and does not have a nitrite reductase. Under conditions of prolonged non-bathing, a strain that does not utilize urea may be preferred. Many heterotrophic bacteria cause the hydrolysis of urea into ammonia. In the presence of a substantial biofilm of AAOB, any urea hydrolysis by such bacteria would be accompanied by prompt release of NO and nitrite, both of which would inhibit most heterotrophic bacteria. Some of the degenerative diseases which can be treated by the method of this invention are characterized by excretion of ammonia. End stage kidney failure, liver cirrhosis are characterized by excretion of ammonia. Another advantage of strains utilizing ammonia is that urea is not very stable in solution, and may decompose over time releasing ammonia and raising the pH. For storage considerations, utilization of ammonia may be preferred.

When bathing is done relatively frequently (every few days), the AAOB biofilm does not have time to achieve great thickness before it is removed through bathing. Under those circumstances, the activity of the biofilm will depend on how many bacteria are applied. Under conditions of prolonged non-bathing, the bioflim can build to substantial thickness and limiting the activity of the AAOB may be desired.

The AAOB have simple metabolic needs, NH3 or urea, O2, CO2, and minerals. They have a fairly high need for trace minerals including iron, copper, and zinc. Some strains also utilize cobalt, molybdenum, and manganese. They also need sodium, potassium, calcium, magnesium, chloride, phosphate and sulfate. All of these compounds are available in sweat in ratios not dissimilar to what is typically used in culture media for these bacteria.

Effects of AAOB on Animal Growth

According to another embodiment of the present invention, it is appreciated that-enhanced growth of cattle and the larger size, earlier puberty, and obesity of humans-in industrialized areas are both due to the inhibition of the normal commensal AAOB. Accordingly, one aspect of the invention is an appreciation that animal growth may be augmented by the removal of AAOB. As used herein, the term “augment” is used to define as an increase in weight, height, width, growth rate, and/or feed efficiency (weight gain per pound of feed). An interesting parallel can be made with animals that are raised for food. Many thousands of tons of antibiotics are incorporated into animal feed to increase growth rate and to increase feed efficiency. There is as yet, no good explanation of the mechanism by which antibiotics stimulate growth. According to McEwen, “the mechanisms of growth promotion are still not exactly known” (Scott A. McEwen and Paula J. Fedorka-Cray. (McEwen and Fedorka-Cray, Antimicrobial Use and Resistance in Animals, Clinical Infectious Diseases 2002; 34(Suppl 3):S93-106.) It has been suggested that they treat a “subclinical infection”, or through the suppression of bacteria that would otherwise consume “nutrients”, or by reducing nutrient consumption by the “immune system”. These mechanisms seem implausible. A “subclinical infection” would be resolved by treatment, and continuous feeding of antibiotics would not be necessary. It would be surprising if every animal in a herd had the same “subclinical infection” and so each was helped to gain weight by the same amount. Similarly, is the immune system of every animal in a herd so over stimulated that they do not gain weight at an optimum rate? As for bacteria consuming nutrients, usually, animals are free to consume as much feed as they want. If bacterial consumption was a few percent higher, the animal could compensate by ingesting more, yet they do not. Also, antibiotic treatment does not render the digestive system of these animals bacteria free. On the contrary, populations of bacteria are still extremely high. Also, many bacteria develop resistance to these antibiotics and persist at high levels.

The growth enhancing properties of antibiotics in feed may be mediated through inhibition of autotrophic ammonia oxidizing bacteria (AAOB) living on the external skin of these animals. In the wild, all animals which sweat (which includes all mammals) would be expected to have a population of ammonia oxidizing bacteria on their external skin metabolizing the urea in their sweat and producing NO and nitrite. Cattle are no exception. Giving large doses of antibiotics would be expected to result in antibiotics in the animals' sweat, and in the inhibition of any AAOB on the external skin. Inhibition of these bacteria would reduce basal NO levels, increase basal metabolism, increase growth rate, increase adult size, shorten the time to maturity, and increase body mass and body fat. These are exactly the changes that have been observed in human populations during industrialization. People get bigger, mature earlier, and become obese.

With this understanding, antibiotics in feed may not be necessary to inhibit AAOB on the external skin. A number of aspects of animal growth enhancement with antibiotics becomes understandable when it is recognized that AAOB are the target organism. AAOB have very small genomes. Nitrosomonas europaea has only 2,460 protein coding genes. It does not have genes for metabolizing xenobiotic compounds. It also does not have membrane transporters to excrete xenobiotic compounds. As an autotrophic bacterium it has a very slow metabolism, with a doubling time 30 times longer than that of heterotrophic bacteria. It would be expected to evolve 30 times slower, but since it also has such a limited genome, it doesn't have the genes which can mutate and then perform new functions such as provide antibiotic resistance. Thus autotrophic bacteria would be expected to evolve antibiotic resistance much more slowly (if at all) than heterotrophic bacteria. Halling-SØrensen has reported that AAOB are gram negative bacteria and are quite sensitive to many antibiotics. (Halling-Sørensen, Inhibition of Aerobic Growth and Nitrification of Bacteria in Sewage Sludge by Antibacterial Agents, Arch. Environ. Contam. Toxicol. 40, 451-460 (2001). Many of the antibiotics used in animal feed are not absorbed, but are excreted in the feces and accumulate in manure. Manure contains abundant ammonia and urea and would in the absence of inhibitory compounds contain an abundance of AAOB. With antibiotics in animal manure, AAOB cannot grow, and so cannot inoculate the external skin of cattle. Using cattle as agents to mix antibiotics with manure and to apply it to their living areas would seem a less than ideal method. According to the present invention, compounds to inhibit AAOB in the animal's living space could be applied directly.

AAOB are quite sensitive to compounds that inhibit the ammonia monooxygenase enzyme. Allylthiourea is such a compound that is very effective at inhibiting ammonia monooxygenase and this compound is commonly used in waste water testing when determining biological O2 demand, or BOD. Allylthiourea is added to inhibit the AAOB which would otherwise oxidize ammonia with O2 and raise-the measured O2 consumption. Nitrification inhibitors are also used in fertilizer utilization. Many plants can absorb nitrogen both as ammonia and as nitrate. However, for nitrogen to be incorporated into an amino acid, it must be in the ammonia form. Nitrate must therefore be reduced to ammonia. This reduction consumes energy that could otherwise be used to make plant biomass. It is therefore desirable in some instances to inhibit the nitrification bacteria in the soil when nitrogen fertilizer is added in the form of ammonia or urea. A number of compounds are in common use in the fertilizer practice, and the use of any of these compounds would also be effective in blocking the nitrification of the urea in sweat when applied topically to the external surface of farm animals.

However, the safety of applying such compounds to animals is unknown. A better approach is to use an anionic detergent. Brandt et al. reported that AAOB are quite sensitive to anionic detergents, and are especially sensitive to linear alkylbenzene sulfonates (LAS) such as 4-(2-dodecyl)benzenesulfonic acid which has been shown to have a 50% inhibitory concentration (IC50) of 5, 3, 1, and 1 mg/L (ppm) for N. europaea, N. mobilis, N, multiformis, Nitrosospira sp. strain AV respectively. (Brandt et al., Toxic Effects of Linear Alkylbenzene Sulfonate on Metabolic Activity, Growth Rate, and Microcolony Formation of 4 Nitrosomonas and Nitrosospira Strains, Applied And Environmental Microbiology, June 2001, Vol. 67, No. 6, p. 2489-2498.) They found that the AAOB tested did not develop resistance or tolerance when exposed to lower doses. The critical micelle concentration (CMC) for LAS is 410 ppm, which is far above the IC50 indicating a chemical effect rather than a detergency mediated effect. Although not bound by one particular theory, a possible reason anionic detergents are so toxic to the AAOB is that as anions, they are ported into the cell by the anion transporter which is necessary to bring in sulfate, phosphate and bicarbonate. Once inside, the AAOB doesn't have the metabolic machinery to get rid of it, either by metabolizing it into innocuous compounds, or to excrete it. Heterotrophic bacteria easily adapt to high levels of LAS and many of them can utilize LAS as a carbon source. LAS is a common anionic detergent used in many cleaning products including dishwashing and laundry detergents though usually not shampoos because it is a little “harsh” and leaves the skin feeling “sticky.” However, LAS is a high volume material with worldwide production in 1987 of 1.8 million tons. Huge quantities are already discharged into the environment, so using it to inhibit AAOB on the skin of farm animals would not be expected to have any environmental impact. In any case, using LAS for farm animal growth enhancement would displace the antibiotics which are already being used and which are already a far worse problem due to induction of antibiotic resistance in pathogenic bacteria. There is extensive data on the safety and irritancy of LAS, but most studies do not look at concentrations far below the CMC, likely because the effects there are so small. In practice, the detergent solution could be sprayed on the animal, and then not rinsed off, or the animal would be forced to swim through a bath of the material. The detergency of a surfactant is approximately constant above the CMC, and approximately linear with concentration below the CMC. Most of the adverse effects of detergents on the skin are due to protein denaturing and defatting of the skin. Because detergency is not required for inhibition of AAOB, levels that denature proteins and defat the skin are not required. One way to ensure a long term inhibitory dosage on the skin is to form a low solubility “soap” in situ. A solution of LAS in water is sprayed on the animal, and then a solution of a divalent salt, such as calcium chloride is sprayed on as well. Mixing would occur on the skin, where the LAS would precipitate as the relatively insoluble calcium LAS soap. The precipitated soap would adhere to the animal's hair and so provide a reservoir of LAS which would dissolve as the animal sweated or was rained upon. The amount of precipitated LAS could be adjusted to attain an inhibitory level of LAS between treatments. The solubility product Ksp for LAS (carbon number ˜12, average MW=343) is 8.4e-12. The calcium content of human sweat is 3 mM/L. Assuming a similar value, for cattle sweat, then at the solubility limit of Ca(LAS)2, the LAS concentration would be 18 ppm. This is sufficiently high that AAOB would be substantially inhibited so long as there was any residual Ca(LAS)2 soap present on the cattle. The initial concentration would be much higher when the detergent is first sprayed on. Other molecular weight LAS compounds have different Ksp's. For example, an LAS with a MW of 339 (carbon number ˜11.4) has a Ksp of 1.8 e-11. This represents a concentration of 26 ppm.

Other inhibitors may be used, but there are few materials as cheap and as benign and as readily available as LAS.

Nitric Oxide Metabolism:

Nitric oxide is produced in the gut by reduction of dietary and salivary nitrate by heterotrophic bacteria. This reduction occurs in two steps, first to nitrite by nitrate reductase and then to nitric oxide by nitrite reductase. Milk contains abundant xanthine oxidoreductase which can also catalyze the reduction of nitrate and nitrite to NO as reported by Ben L. J. Godber, et al. (Godber et al., Reduction of Nitrite to Nitric Oxide Catalyzed by Xanthine Oxidoreductase, The Journal Of Biological Chemistry, Vol. 275, No. 11, Issue of March 17, pp. 7757-7763, 2000.) Excessive NO from this route can cause “blue baby” syndrome which results from oxidation of blood hemoglobin to methemoglobin. Methemoglobin is not toxic, however it does not carry O2 and in excessive quantities can cause hypoxia. T. Ljung et al showed that nitric oxide is produced in the gut by children with active inflammatory bowel disease, where rectal NO was increased approximately 100 fold over that of healthy children. (Tryggve Ljung et al., Increased rectal nitric oxide in children with active inflammatory bowel disease, J Pediatric Gastroenterology and Nutrition, 34:302-306, 2002.) Fecal NO was not increased over that of healthy children, implicating a source other than bacterially generated NO (however, as their assay method appeared to be aerobic, it may not have detected the anaerobic NO production expected from bacterial nitrite reductase). An increased NO observed during inflammatory bowel disease may be an adaptive reaction to low basal NO levels.

E. Weitzberg et al. have reported that humming increases NO production in the nasal passages. (Eddie Weitzberg et al., Humming greatly increases nasal nitric oxide, Am J Resp Crit Care Medicine Vol 166. 144-145 (2002).) The NO production is limited by diffusion of O₂ to the active enzyme. Humming increases the gas exchange and so increases NO production and NO measured in nasal air. The NO in the air is inhaled, but most of it would be oxidized to nitrate in the lung. However, the concentration of NO at the site of generation is higher, and some may diffuse into the blood supplying the nasal passage, which drains into the various sinuses in the brain. Humming, which is an observed characteristic behavior of some autistic individuals, may increase NO levels.

R. Henningsson et al have shown that chronic inhibition of NOS with L-NAME in mice unexpectedly increases total pancreatic islet NO production. (Ragnar Henningsson et al., Chronic blockade of NO synthase paradoxically increases islet NO production and modulates islet hormone release, Am J Physiol Endocrinol Metab 279: E95-E107, 2000.) However, the regulation of NO synthesis is exceedingly complex. Of all the normal metabolic products, NO is one that inhibits respiration. Sufficiently high NO levels will shut down respiration and can cause cell damage. NO is part of the mechanism by which foreign cells are killed, so immune cells may have the capacity to generate cytotoxic levels of NO. Cytotoxic levels of NO cannot be regulated at the source of NO because cells there are killed. Therefore, the regulation may be separated in time or space from the site of NO generation. Inducible NOS may separate the regulation of high NO production in time. Separation in space may require a different (as yet unknown) messenger molecule.

NO is produced in response to activation of many different receptors. For example, K. Chanbliss has shown that an estrogen receptor causes the release of NO, (Ken L. Chambliss et al., Estrogen modulation of endothelial nitric oxide synthase. Endocrine reviews 23(5):665-686.) P. Forte has demonstrated that women are observed to have higher levels of NO metabolites, and reduced incidence of diseases associated with low nitric oxide, including hypertension and cardiovascular disease (Pablo Forte et al., Evidence for a difference in nitric oxide biosynthesis between healthy women and men. Hypertension, 1998;32:730-734.) The different incidence of autism between males and females may derive from an increased basal NO level in females due to increased estrogen mediated NO release.

Nitric Oxide and Stress

NO tonally inhibits cytochrome oxidase by competitive inhibition with O₂. This inhibition has important physiological effects, in that the delivery of O₂ to individual mitochondria is by purely passive diffusion. Were there no regulation of O₂ consumption, the mitochondria closest to the O₂ source may consume the most O₂, and mitochondria farther away may get less or none. Competitive inhibition with NO, may allow the metabolic load to be distributed over many mitochondria. This may be important in tissues where the O₂ consumption is highly variable, such as in muscle. The O₂ consumption of heart muscle can vary by nearly an order of magnitude. Because O₂ delivery is by passive diffusion, and the geometry of the source and sink doesn't change (there is some increased vascular recruitment, but not an-order-of magnitude) and the O₂ source (partial pressure of O₂ in the vasculature) doesn't change much, that when the O₂ flux changes by an order of magnitude, the O₂ gradient may change to produce the increased driving force for O₂ diffusion. The O₂ concentration at the mitochondria under conditions of high O₂ consumption may be less in order for more O₂ to diffuse there. To increase the O₂ flux an order of magnitude at constant source and geometry, the O₂ sink concentration may drop an order of magnitude. If the O₂ consumption increases an order of magnitude while the concentration drops an order of magnitude, the enzyme activity may increase two orders of magnitude. In order to increase metabolic capacity, NO levels may be reduced. This is the “feature” of superoxide production during hypoxia. Superoxide destroys NO and so disinhibits the mitochondria O₂ consumption, allowing mitochondria to consume O₂ even at very low O₂ concentrations. The very low O₂ concentration may allow O₂ to diffuse to where it is being consumed. Superoxide is undesirable, because it damages proteins. However, not enough ATP is worse because then the cell doesn't have the capacity to respond and will necrose.

Nitric Oxide Regulation and Feedback:

NO is generated at diverse sites and then diff-uses to diverse other sites where the action of NO is exerted through diverse mechanisms. While NO is a rapidly diffusing gas, and has a “short” diffusion path length, each site may integrate the total NO signal that it receives. A reduction in the basal nitric oxide level may reduce the background level of NO. A reduced background level of NO may result in a decrease in the effective range of NO produced as a second messenger. With a lower background level, the transient NO source may activate a downstream target, may be more diluted and so may have a shorter range at which it reached activating concentrations. It is this shorter range of action that may be important in the malformation of neural connections. The migrating axons may not get “close enough” to receive the NO signal that they need to “home in” on. Axons that do get “close enough” do make good high density local connections, and may perhaps be the explanation for increased aural discrimination.

When an NO source is part of a feedback loop, that source may then be regulated to produce higher levels of NO, which may compensate for the lower background level. The concentration at the NO source to achieve the regulated level after diff-using to the NO sensor may be higher, and may be much higher than with a higher background level. Cells closer to the source than the NO sensor may then be exposed to higher NO levels than “normal.” Cells farther away from the source than the NO sensor may be exposed to lower NO levels.

Virtually all important metabolic systems are under some type of feedback control. Nitric oxide may be involved in many feedback control loops, including the regulation of peripheral vascular resistance by shear stress dependant NO release followed by vessel dilatation. A difficulty with the feedback control of NO is that NO diffuses readily, and it has a short half life. A source of NO may produce an NO concentration higher than the sink which consumes it. Nitric oxide is toxic at high levels, and any source of nitric oxide must be regulated, either in time, by feedback, or in space. If basal NO concentration is regulated by feedback, inhibition of some sources may cause other sources to be up-regulated. The observation that autistic children have higher levels of NO metabolites may also be explained by not enough NO in the right place, so more NO is produced to compensate.

For example, the hypotension of septic shock is largely from the excess production of nitric oxide by iNOS. iNOS is the inducible form of NOS, and is an example of a “feed forward” type of control, rather than a “feed back” kind of control as in eNOS. The production of very high levels of nitric oxide by cells is best achieved by a “feed forward” type of control. Once a cell starts to produce high levels of nitric oxide, the nitric oxide so produced may inhibit the cytochrome oxidase of the mitochondria in those cells and will interfere with normal cell metabolism.

G. Stefano et al. have shown that the production of basal nitric oxide by human granulocytes has been shown to be time periodic, with a period of a few minutes, and in the 1000 pM range. (George B. Stefano, et al., Cyclic nitric oxide release by human granulocytes and invertebrate ganglia and immunocytes: nano-technological enhancement of amperometric nitric oxide determination, Med Sci Monit, 2002;8(6): BRI 99-204.) These measurements were done 10 μm above a pellet of 10E3 cells. This periodic signal was necessarily an average from many cells. That a periodic signal was observed indicates that the cells were producing NO at a time varying rate, and that this NO production was in phase. Maintaining phase coherence over so many cells would indicate communication between cells, and feedback control of NO release. It is possible that some other messenger molecule mediates the communication between cells, however any such molecule would need to have a shorter lifetime and more rapid diffusion than NO in order to maintain phase coherence. However, there may be direct sensing of nitric oxide concentration, and feedback regulation of nitric oxide production, albeit with a time lag.

Basal NO levels cannot be measured and regulated at the site of NO production because the site of NO production is necessarily above basal levels. NO must be measured remotely and the signal transmitted through a non-NO transmitter to the cells that are producing the basal NO.

An “exercise” hypothesis would argue that since nitric oxide is produced in response to physical activity, humans may have evolved to rely upon the nitric oxide produced by the moderate physical activity needed for a hunter-gatherer lifestyle. “Normal” physical activity levels may have produced sufficient nitric oxide, and so there was may have been no evolutionary pressure to evolve other nitric oxide sources. However, prehistoric infants and toddlers were not hunter gatherers. Their food was hunted and gathered by their caretakers who may well have been more physically active than modern caretakers. The physical activity level of pre-crawling or pre-walking children may not have been much higher in prehistoric times. However, an unrecognized source of nitric oxide upon which humans relied during prehistory may be that of the commensal autotrophic ammonia oxidizing bacteria, and that the frequent bathing of a modern lifestyle removes this source of nitric oxide.

Autotrophic Ammonia Oxidizing Bacteria as a Source of NO:

Commensal autotrophic ammonia oxidizing bacteria present on the skin and in particular on the scalp to generate physiologic NO from the urea in sweat, provides a rational for sweat excretion other than as a cooling mechanism. Adrenergic sweating occurs during stimulation of the adrenergic system. Adrenergic sweating occurs during periods of stress and also commonly occurs at night. It may be that sweating on the scalp at night may serve to administer a fairly high dose of NO to the brain and to thereby “reset” the NO signaling pathways and allow the brain to do all the “housekeeping” functions that require high NO levels.

These bacteria have not been identified as associated with the human body because they do not cause any disease. In fact, they likely cannot cause disease (probably not even in immunocompromised individuals). From an inspection of the genome, it is clear that these bacteria cannot cause disease. There are no genes for toxins or lytic enzymes. They do not have the metabolic machinery to utilize the complex organic compounds such as are found in animal tissues. As autotrophic bacteria, they are incapable of growing anywhere that lacks the substrates they require, ammonia or urea, O2, mineral salts. These substrates may be abundantly available on the unwashed skin from sweat residues, and in the “wild” and in the absence of frequent bathing with soap, humans would be unable to prevent the colonization of their external skin with these bacteria. These bacteria may be beneficial and commensal, and that many aspects of human physiology may have evolved to facilitate the growth of these bacteria and the utilization of the NO they so abundantly produce.

Another factor that perhaps has prevented their isolation may be the bathing practices in developed regions. It has become customary to bath with sufficient frequency so as to prevent the development of body odor. Body odor generally occurs after a few days of not bathing, and the odor compounds are generated by heterotrophic bacteria on the external skin which metabolize exfoliated skin and sweat residues into odiferous compounds. In 3 days, autotrophic bacteria could double approximately 7 times for approximately a 100-fold increase over the post bathing population. In contrast, heterotrophic bacteria could double approximately 200 times for a 10e+60-fold increase. Heterotrophic bacterial growth would be nutrient limited. Assuming similar kinetics of removal through bathing of autotrophic and heterotrophic bacteria, controlling heterotrophic bacteria though bathing would reduce autotrophic bacteria to low, perhaps undetectable levels.

The inventor has found that a sufficient population of AAOB on the skin substantially suppresses body odor due to heterotrophic bacteria. The inventor has applied AAOB to his skin and has refrained from bathing for >2 years now, including three summers. There is essentially no body odor associated with sweating. In fact, sweating decreases body odor by nourishing the AAOB and enhancing their production of NO and nitrite. During the winter, with decreased sweating due to low ambient temperatures, there was an increase in odor. However, with increased clothing, (wearing sweaters) the inventor was able to increase basal sweating and reduce body odor to near zero again. There has been no itching, no rashes, no skin infections, no athlete's foot infection, and substantially no foot odor.

L Poughon et al. have reported that AAOB produce nitric oxide as an intermediate in their normal metabolism. (Laurent Poughon, et al., Energy Model and Metabolic Flux Analysis for Autotrophic Nitrifiers. Biotechnol Bioeng 72: 416-433, 2001.) D. Zart et al. have demonstrated one strain had optimum growth at concentrations of NO in air around 100 ppm (highest level tested in this study). (Dirk Zart, et al., Significance of gaseous NO for ammonia oxidation by Nitrosomonas eutropha, Antonie van Leeuwenhoek 77: 49-55, 2000.) AAOB can tolerate higher levels. I. Schmidt has shown that with other strains, there was no decline in NH3 consumption from 0 to 600 ppm (anaerobic in Ar plus CO₂) but it declined by ⅓ at 1000 ppm NO. (Ingo Schmidt et al., Anaerobic Ammonia Oxidation in the Presence of Nitrogen Oxides (NOx) by Two Different Lithotrophs, Applied And Environmental Microbiology, November 2002, p. 5351-5357. ) Most AAOB are aerobic, but some strains can utilize nitrite or nitrate in addition to O2 which increases the NO production. 1000 ppm NO in air corresponds to about 2 μM/L in aqueous solution. The strain used by the inventor has produced a measured NO concentration of 2.2 μM/L. Most studies of AAOB metabolism have been motivated by their utilization in waste water treatment processes for ammonia and nitrate removal from waste water. Operation of waste water treatment facilities at hundreds of ppm NO is undesirable, so it is not unexpected that the physiology of these bacteria under those conditions has not been well studied.

The inventor has noticed that a number of characteristics which may be associated with Asperger's have changed since applying these bacteria. It has become more difficult to “multi-task”. Stimuli are more distracting, that is it is not as easy as it used to be to work while distracting stimuli are present. However, learning new information is easier, and that information is better integrated with previous information.

Subjectively, the sleeping pattern of the inventor has subjectively changed, in that he now awakes less frequently during the night. The inventor's senses of smell and touch have subjectively become more acute, and threshold stress for joint pain has seemingly decreased. These changes while subjective are consistent with increased NO levels. The inventor and others have noticed that dreams are more vivid after application of these bacteria to the scalp demonstrating an affect of increased NO on a normal neurological process.

Experimental: Pilot Study (n of 1):

An enrichment culture of AAOB was prepared from barnyard soil using NH₄Cl in organic-free media simulating human sweat. After a number of passages and growth to high mM nitrite levels (to attenuate heterotrophic bacteria) the AAOB culture was applied to the scalp of a subject (now 49 year old male). Continuous growth has now persisted for 33 months and an active AAOB biofilm has accumulated, nourished solely from natural secretions. After 5 months, the culture was applied to the subject's entire body. So as to simulate conditions in the “wild”, bathing was stopped. Surprisingly, body odor has not developed, even after over 27 months of non-bathing, even after profuse thermal and exercise induced sweating. There was a slight increase in odor during the first winter when sweating diminished due to lower ambient temperatures. However, the wearing of sweaters increased basal sweating and promptly decreased odor.

It may be that NO, nitrite, NO₂ (which can sometimes be detected by smell), and perhaps NO adducts produced by these AAOB must be suppressing the odor-causing heterotrophic bacteria.

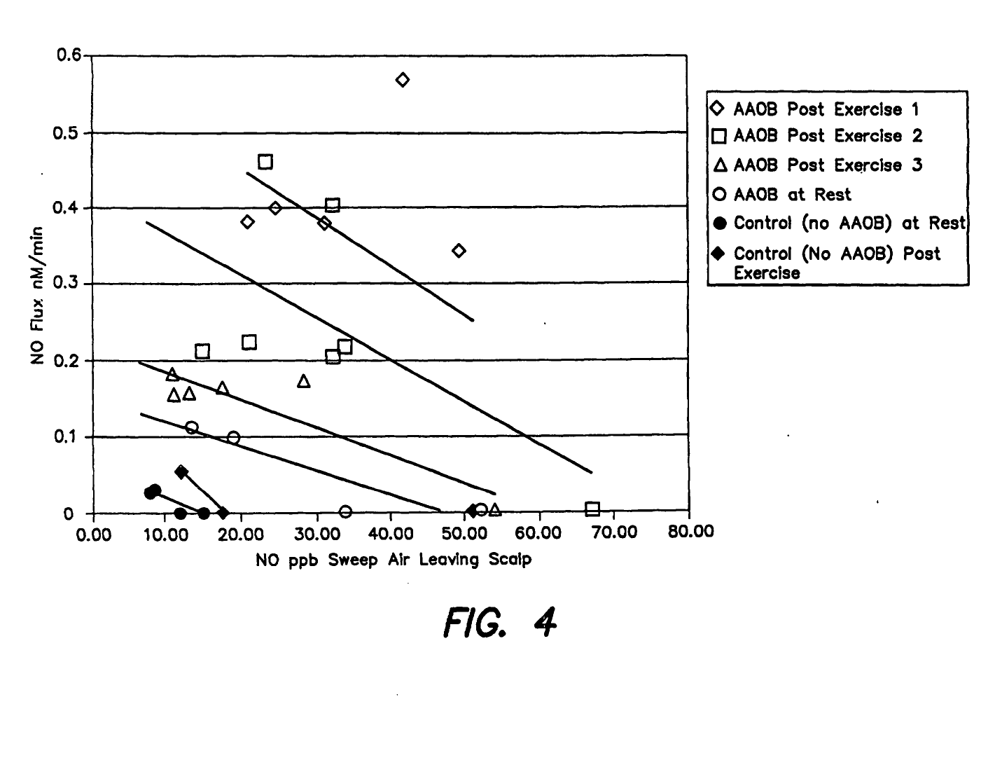

Measurement of the NO produced by the biofilm was undertaken. The scalp was covered with a close fitting cap of PTFE film held in place with an external knitted polyester band (hard hat brim type wind sock), and ambient air drawn past the scalp, through a gas flow meter (Omega FMA1816), and then sampled with a NO analyzer (Sievers NOA 280i). Flow and NO were recorded ˜1/sec. NO flux verses NO in the sweep gas was plotted in FIG. 4. At higher flow rates, the NO concentration went down, but the flux went up. The NO flux was generated by the AAOB biofilm and diff-used both into the air under the cap where it could be measured and into the scalp where it could not be measured. However, the NO source could not change as rapidly as the external gas flow could be changed so by rapidly changing the external diffusion resistance the internal flux could be inferred. The “NO source”, is the “intercept”, it is the NO flux at zero external concentration. The “zero flux” point is measured and is the concentration reached when external diffusion is blocked (peak NO measured with resumed flow).

The NO flux leaving the scalp with accumulated AAOB biofilm is substantial, approaching 1 nM/min after a period of exercise. After exercise, the flux was changing rapidly, so there is some scatter when trying to fit it to a straight line. The NO flux into the scalp inferred from these measurements is substantial, ˜0.3 nM/minute. With the same apparatus, a similar subject (male age 48) without these bacteria (control) had a much smaller measured NO flux (0.03). An increase in NO is observed in the post exercise period, however, the basal NO level observed in the colonized individual is significantly greater than the post exercise stimulated NO level of the uncolonized individual.

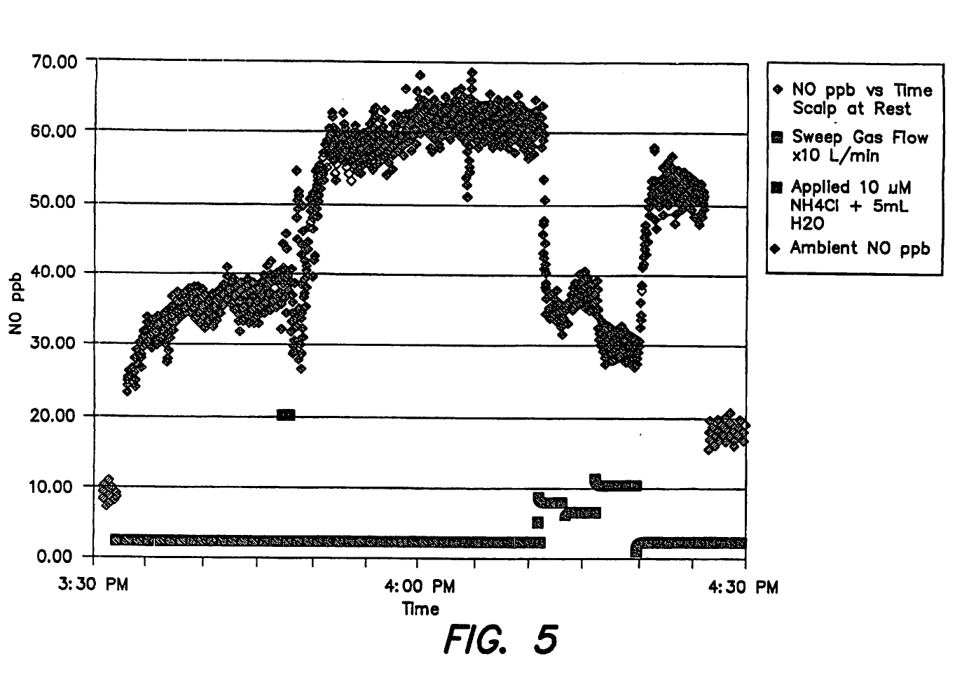

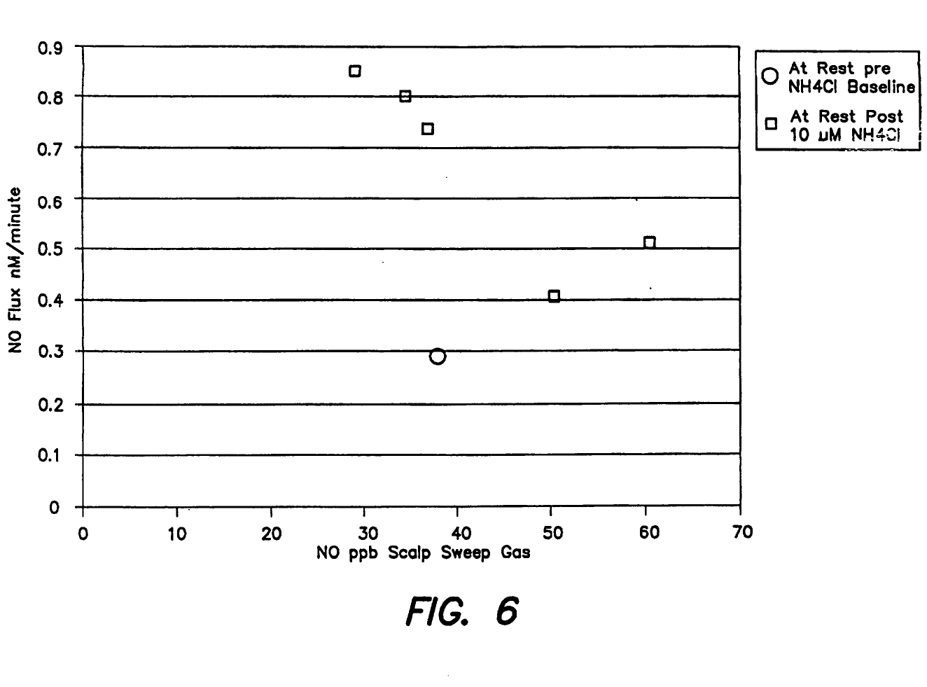

In another series of experiments, 10 μM NH₄Cl in 5 mL H₂O was applied to the scalp. FIG. 5. is a continuous trace of NO concentration of the sweep gas. the 10 μM NH₄Cl in 5 mL H₂O was applied by snaking a tube under the PTFE cap. The resultant NO flux is illustrated in FIG. 6. The NO flux promptly increased (from 0.3 to 0.8 mM/min in ˜1 minute), demonstrating that the NO is derived from NH₃ and not from nitrite or nitrate or mammalian nitric oxide synthase. The promptness of the increase demonstrates that NO release is closely coupled to NH₃ release by sweat. The particular strain of AAOB used in the present experiments does not utilize urea directly only NH₃ and it does not have a nitrite reductase.

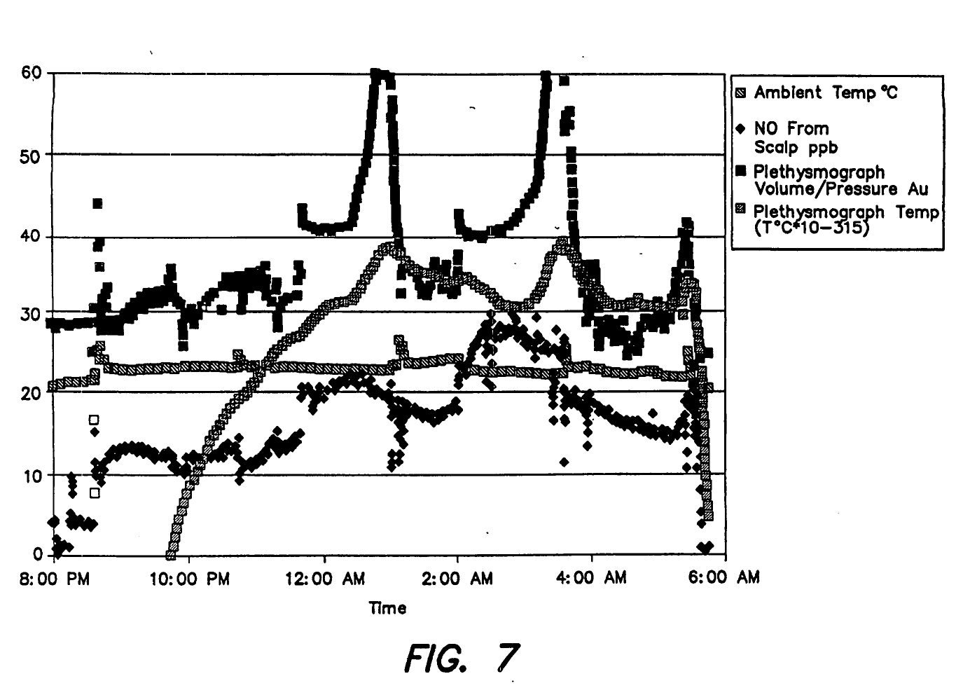

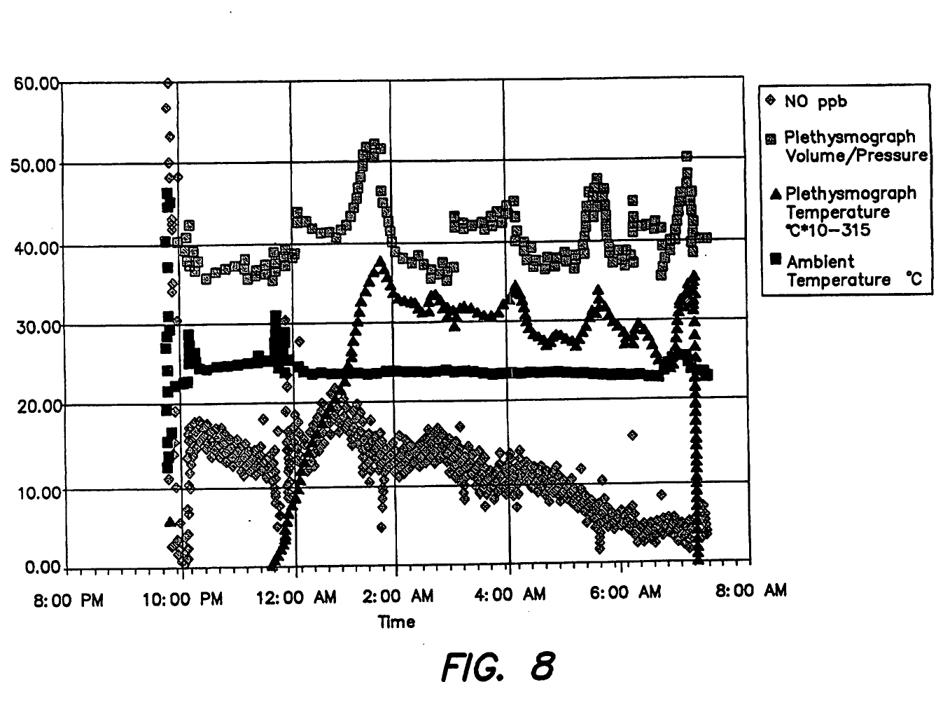

The PTFE cap was applied and continuous NO measurements taken during otherwise normal sleep. A plethysmograph was used to monitor tumescence via pressure (volume) and temperature (blood flow). Measurement of NO and plethysmograph pressure and temperature were recorded every ˜10 seconds, as shown in FIGS. 7 and 8. In tests on 4 consecutive nights there were 11 instances of nocturnal erection and 6 increases in NO flux increase, immediately prior to or coincident with the increase in tumescence. The traces are from the first night which shows two instances of the most compelling association between NO release and tumescence, and from the last night which shows 4 instances of tumescence. Whether this increase in NO is causal or is simply associated with sweating which preceded and accompanied the tumescence is unknown. Increased nocturnal erection was subjectively noticed after first applying the AAOB and this has continued unabated now for >2 years. NO is known to be important in erection physiology. A common folk remedy for impotence is application of saliva to the penis. Saliva contains nitrite from reduction of salivary nitrate by heterotrophic bacteria on the tongue. Skin contains xanthine oxidoreductase which reduces nitrite to NO. Topical application of NO donors is used as a treatment for erectile dysfunction.

Production of NO by AAOB, closely coupled to the supply of ammonia, and inhibition of heterotrophic bacteria on the skin is demonstrated. It would be surprising if over evolutionary time, such a source of NO species would not be incorporated into normal human physiology. NO release was observed coincident with physiological effects known to be mediated via NO. It may be that a physiologic explanation for adrenergic sweating is to supply ammonia to a resident biofilm of AAOB for prompt release of nitrite and NO. The profuse sweating observed in many disorders may be a normal physiologic response to nitropenia.

As NO emitters, AAOB may be somewhat resistant to attack by the immune system due to suppression of inflammation via inhibition of NFκB. As a commensal non-pathogenic organism present on the skin over evolutionary time scales, the immune system may have evolved to allow their presence. Some AAOB are motile, and migration into and colonization of sweat pores might be advantageous to both the bacteria and humans. It would shorten the diffusion distance for NO absorption, and would reduce potential colonization by heterotrophic bacteria and fungi. While AAOB are aerobic, they can tolerate low O₂ levels, and can actively respire at ˜12 Torr O₂ as reported by Ruiz et al. (Nitrification with high nitrite accumulation for the treatment of wastewater with high ammonia concentration. Water Res. March 2003;37(6):1371-7. ˜12 Torr is lower than the minimum O₂ level measured in the skin. Colonization of the pores might protect AAOB from light, washing and casual bathing, however, the increasingly common practice of frequent bathing with anionic detergents and antimicrobial agents may be more than they can tolerate.

Hard and Soft Water:

Living in regions with hard water (water with Ca and Mg ions) has been correlated with lower incidences of a number of diseases including stroke, cardiovascular disease, and diabetes. Magnesium in drinking water and the risk of death from diabetes mellitus and even cancer. Calcium and magnesium in drinking water and the risk of death from breast cancer. (J Toxicol Environ Health A. June 2000;60(4):231-41.) Health effects from hard water have generally been attributed to either a positive effect of increased ingestion of Ca and Mg or a lessened toxic effect due to reduced leaching of Cd or other heavy metals. However, Ca and Mg from other dietary sources doesn't have the same effect. (Nerbrand C, Agreus L, Lenner R A, Nyberg P, Svardsudd K., The influence of calcium and magnesium in drinking water and diet on cardiovascular risk factor in individuals living in hard and soft water areas with differences in cardiovascular mortality, BMC Public Health. Jun. 18 2003). Drinking is not the only use of domestic water. Generally domestic water is used for both drinking and bathing. Hard water is difficult to bathe with because the divalent ions form insoluble soap precipitates, leaving the soap unavailable as a surfactant. Bathing with soap and even detergents is less effective in hard water. Because hard water precipitates many anionic surfactants, hard water reduces the toxicity of surfactants on many species (Coral Verge, Alfonso Moreno, Jose Bravo, Jose L. Berna, Influence of water hardness on the bioavailability and toxicity of linear alkylbenzene sulfonate (LAS), Chemosphere 44 (2001) 1749-1757). On human skin, hard water would hinder removal of an AAOB biofilm, would reduce the toxicity of soap and detergents toward AAOB, and might reduce the motivation for bathing, particularly the motivation for washing one's hair.

A negative correlation between water hardness and ischemic heart disease mortality was observed in the Netherlands, with correlation coefficients of declining significance, from 1958-1962, 1965-1970 and 1971-1977. (Zielhuis R L, Haring B J. Water hardness and mortality in the Netherlands, Sci Total Environ. April 1981;18:35-45). Interestingly, this is approximately the same period over which synthetic detergent use increased, and when shampoo technology advanced rapidly. Commensal skin-adapted strains of AAOB are likely able to tolerate saponified fatty acids, likely abundant on unwashed skin. Soap may facilitate their removal along with surface dirt, but is unlikely to exert specific toxic effects. Alkylbenzene sulfonates in contrast are toxic to AAOB at ppm levels.

It may be that the main sites of NO production are places with hair, scalp hair and pubic hair, where the NO and nitrite might serve as a defense against infection. Hair may serve to provide a protective niche for AAOB, and to reduce heat loss through skin which must be thin and well vascularized to facilitate NO absorption. I suspect that the AAOB are under active physiological control. Some health changes have been observed during this pilot study. However, with an n of 1, and without controls, it is difficult to definitively ascribe these health changes solely to increased NO from topical AAOB, and many of the changes observed are subjective.

Subjective health changes observed in pilot study include: appetite reduction and weight loss, increased motivation to exercise, allergy reduction (hay fever), reduction in serum alanine transaminase levels, reduction in blood pressure, more rapid healing of skin wounds, reduction in rate of hair loss/regrowth of lost hair, increased mental acuity and improved mood.

What is claimed is:

1. A method of treating a subject who has developed or is at risk of developing at least one of hypertension, hypertrophic organ degeneration, Raynaud's phenomena, fibrotic organ degeneration, allergies, autoimmune sensitization, end stage renal disease, obesity, diabetes type 1, osteoporosis, impotence, hair loss, cancer, aging, autism, an autism spectrum symptom, retarding due to aging, comprising:
identifying a subject who has developed or is at risk of developing at least one of hypertension, hypertrophic organ degeneration, Raynaud's phenomena, fibrotic organ degeneration, allergies, autoimmune sensitization, end stage renal disease, obesity, diabetes type 1, osteoporosis, impotence, hair loss, cancer, autism, an autism spectrum symptom; and
positioning ammonia oxidizing bacteria in close proximity to the subject.

2. The method of claim 1, wherein the act of positioning the bacteria comprises positioning a bacteria selected from the group consisting of any of Nitrosomonas, Nitrosococcus, Nitrosospira, Nitrosocystis, Nitrosolobus, Nitrosovibrio, and combinations thereof.

3. The method of claim 2, wherein the act of positioning ammonia oxidizing bacteria comprises:
applying ammonia oxidizing bacteria to a surface of the subject in an effective amount to cause the bacteria to metabolize any of ammonia, ammonium salts, or urea on the surface into any of nitric oxide, nitric oxide precursors or combinations thereof.

4. The method of claim 3, wherein the act of applying the bacteria comprises applying the bacteria in a suitable carrier.

5. The method of claim 3, wherein the act of applying the bacteria to a surface comprises applying the bacteria to skin, hair, or a combination thereof.

6. The method of claim 3, wherein the act of applying the bacteria comprises applying a substantially pure bacteria.

7. The method of claim 3, wherein the act of applying the bacteria comprises:
applying the bacteria to an article; and
contacting the article with the surface of the subject.

8. The method of claim 3, wherein the act of applying the bacteria comprises applying the bacteria mixed with an acid.

9. A method of augmenting animal growth comprising:
removing AAOB from the surface of the animal.

10. Use of ammonia oxidizing bacteria in the manufacture of a medicament for providing nitric oxide to a subject, wherein said medicament is suitable for being positioned in close proximity to said subject, substantially as described in the specification, wherein the subject has developed or is at risk of developing at least one of: hypertension, hypertrophic organ degeneration, Raynaud's phenomena, fibrotic organ degeneration, allergies, autoimmune sensitization, end stage renal disease, obesity, diabetes type 1, osteoporosis, impotence, hair loss, cancer, autism, an autism spectrum symptom, and reduced health due to aging.

11. The use of claim 10, wherein said bacteria are selected from the group consisting of any of Nitrosomonas, Nitrosococcus, Nitrosospira, Nitrosocystis, Nitrosolobus, Nitrosovibrio, and combinations thereof.

12. The use of claim 11, wherein said medicament is suitable for application to a surface of the subject in an effective amount so as to cause said bacteria to metabolize any of ammonia, ammonium salts, or urea on the surface into any of nitric oxide, nitric oxide precursors or combinations thereof.

13. The use of claim 12, wherein the medicament is suitable for application to skin, hair, or a combination thereof

14. The use of claim 12, wherein the medicament is suitable for application to an article and wherein the article is suitable for contact with the surface of said subject.
